(19) United States
(12) Patent Application Publication
(10) Pub. No.: US 2011/0202033 A1
(43) Pub. Date: Aug. 18, 2011

(54) COMPOSITIONS AND METHODS FOR THE TREATMENT AND PREVENTION OF CARDIAC ISCHEMIC INJURY

(76) Inventors: Noah Weisleder, Elizabeth, NJ (US); Jianjie Ma, Belle Mead, NJ (US)

(21) Appl. No.: 13/077,449

(22) Filed: Mar. 31, 2011

Related U.S. Application Data
(63) Continuation-in-part of application No. 12/307,303, filed on Jan. 2, 2009.
(60) Provisional application No. 60/830,013, filed on Jul. 11, 2006.
(60) Provisional application No. 60/876,871, filed on Dec. 22, 2006.

(51) Int. Cl. A61M 37/00; A61K 35/12; C12Q 1/02; A61K 38/17; A61K 48/00; A61K 31/685; A61K 33/30; A61K 31/555; A61K 31/7088; A61P 9/10

(52) U.S. Cl. 604/500; 424/93.7; 435/29; 514/16.4; 514/44 R; 514/114; 424/641; 514/188; 514/44 A

(57) ABSTRACT

Disclosed herein are compositions and methods for the treatment and/or prevention of pathological conditions associated with ischemia/reperfusion injury and/or hypoxic injury of myocardial cell or tissue.

### CROSS-REFERENCE TO RELATED APPLICATIONS

This application is a continuation-in-part of U.S. patent application Ser. No. 12/307,303 filed Jan. 2, 2009; which claims the benefit of PCT/US2007/015815, filed Jul. 11, 2007; which claims the benefit of U.S. Provisional Applications Nos. 60/830,013 filed Jul. 11, 2006; and 60/876,871 filed Dec. 22, 2006; and U.S. patent application Ser. No. 12/328,646 filed Dec. 4, 2008; which claims the benefit of U.S. Provisional Application 61/005,410 filed Dec. 4, 2007; the disclosures of which are all incorporated by reference herein in their entirety for all purposes.

### INCORPORATION BY REFERENCE

In compliance with 37 C.F.R. §1.52(e)(5), the sequence information contained in electronic file name: Weisleder2011_ST25.txt; size 57 KB; created on: Mar. 31, 2011; using Patent-In 3.5, and Checker 4.4.0 is hereby incorporated herein by reference in its entirety.

### STATEMENT REGARDING FEDERALLY SPONSORED RESEARCH

The U.S. Government has certain rights in this invention pursuant to the following grants: RO1-HL069000; title “Bidirectional Ca signaling in striated muscles” awarded to Dr. Jianjie Ma by the United States National Institutes of Health (NIH).

### FIELD OF THE INVENTION

This invention relates to compositions and methods of use thereof for the modulation of cardiac function.

### BACKGROUND

To maintain cellular homeostasis, eukaryotic cells must conserve the integrity of their plasma membrane through active recycling and repair in response to various sources of damage. For example, in response to external damage and internal degeneration, the cells of the body must repair the membrane surrounding the each individual cell in order to maintain their function and the health of the organism.

Repair of damage to the plasma membrane is an active and dynamic process that requires several steps, including participation of molecular sensor(s) that can detect acute injury to the plasma membrane, nucleation of intracellular vesicles at the injury site and vesicle fusion to enable membrane patch formation. It has been demonstrated that entry of extracellular calcium is involved in the fusion of intracellular vesicles to the plasma membrane, however, the molecular machinery involved in sensing the damaged membrane signal and the nucleation process for repair-patch formation have not been fully resolved.

Defects in the ability of the cell to repair external membranes have been linked to a broad spectrum of diseases and pathological conditions, for example, neurodegenerative diseases (e.g., Parkinson's Disease, BSE, and Alzheimer's), heart attacks, heart failure, muscular dystrophy, bed sores, diabetic ulcers, oxidative damage, and tissue damage such as sinusitis that occurs as side effect from the administration of chemotherapeutic agents. Also, the muscle weakness and atrophy associated with various diseases, as well as the normal aging process, has been linked to altered membrane repair. In order for these cells to repair their membranes in response to acute damage they make use of small packets of membrane that are inside of the cell, referred to as vesicles. These vesicles are normally found within the cell, but upon damage to the cell membrane, these vesicles move to the damage site and form a patch to maintain the cell integrity. Without this essential function, the cell can die and the cumulative effect of this cellular injury can eventually result in dysfunction of the tissue or organ.

Ischemic heart disease caused by coronary atherosclerosis remains the single greatest cause of mortality in western countries and is the predicted number one killer worldwide in 2020¹. As a result of atherosclerosis or cardiac surgery, blockage of heart blood flow leads to acute myocardial infarction that produces two distinct types of mysocardial damage, including ischemic injury induced by the initial loss of blood flow, and reperfusion injury by the restoration of oxygenated blood flow. Although the myocardium can tolerate brief exposure to ischemia (around 20 minutes) by switching metabolism to anaerobic glycolysis and fatty acid utilization and reducing contractility, persistent ischemia results in irreversible myocardial damage, leading to profound myocyte death and a permanent loss of contractile mass. Timely reperfusion of ischemic heart is the only way to preserve cardiac cell viability. However, reperfusion can trigger further damage to the myocardium, i.e., ischemia/reperfusion (IR) injury, via reactive oxygen species (ROS)-induced oxidative stress, induction of the mitochondrial permeability transition pore (MPEP), hyper-contraction, and apoptotic and necrotic heart muscle cell death. Thus, both persistent ischemic injury and IR injury represent important therapeutic targets.

While surgical or pharmacological interventions are clinically used to reseestablish heart blood flow and treat arrhythmias and remodeling associated with infarction, surprisingly no treatment is currently available to prevent or alleviate IR-induced myocardial damage, particularly cardiomyocyte injury and death. Since mammalian cardiamyocytes irreversibly withdrawn from the cell cycle soon after birth and undergo terminal differentiation, preservation of cariomysocytes is crucial for a favorable outcome of post-MI patients. The search for interventions that protects the heart against IR injury has fascinated biomedical researchers for more than two decades, and led to the discovery of ischemic preconditioning (IPC), i.e., nonlethal ischemic stress to the heart (IPC) protects against subsequent lethal myocardial ischemia/reperfusion injury. To date, IPC is the most effective intrinsic cellular mechanism to protect multiple organs including heart, brain, liver, and kidney from ischemia/reperfusion injury.

Accordingly, there exists an ongoing need for the development of pharmaceutical modulators of the processes for the treatment and/or prevention of cardiac damage related to ischemia and reperfusion injury.

### SUMMARY

The present invention relates to the surprising and unexpected discovery of proteins and processes involved in the protection of myocardial cells and/or tissue from damage due to cardiovascular diseases and/or cardiac ischemia/reperfusion injury, hypoxic injury, heart failure, or any combination thereof.

In particular, presently described are nucleic acids, and polypeptides encoded from nucleic acids of the invention, which, alone or in combination with other components, can modulate cardiac function. Also described are compositions, for example, polypeptides, nucleic acids encoding cytoplasmic, nuclear, membrane bound, and secreted polypeptides; as well as vectors, host cells, antibodies, recombinant proteins, pseudopeptides, fusion proteins, chemical compounds, and methods for producing the same.

In certain aspects, the present invention also relates to compositions useful as therapeutics for treating and/or prevention of cardiac cell and/or tissue damage due to cardiovascular diseases and/or cardiac ischemia/reperfusion injury, hypoxic injury, heart failure, or any combination thereof.

Therapeutic compositions of the invention comprise MG53 polypeptides, and nucleic acids encoding MG53 polypeptides, for example, the protein of SEQ ID NO. 1 and MG53 receptor polypeptides, and mutants, homologs, fragments, truncations, pseudopeptides, peptide analogs, and peptidomimetics (herein, “MG53 polypeptides”), as well as proteins and compounds that can modulate the activity of MG53 or intermolecular interactions of MG53 with MG53 receptor polypeptides, for example, CSN6, kinesin, caveolin-3 (SEQ ID NO. 8), periaxin, phosphoinositide-3 kinase (PI3K) and myelin-basic-protein. As described herein, MG53 functions as an important modulator of the protective response of cardiovascular diseases and/or cardiac ischemia/reperfusion injury, hypoxic injury, heart failure, or any combination thereof, and therefore, the targeting and modulating MG53 gene expression, polypeptide synthesis, activity or protein-protein interactions represent a novel therapeutic intervention for the treatment and/or prevention of IR injury.

In further aspects, the invention relates to compositions comprising a polypeptide of the invention in combination with an agent that exerts its effects, synergistically, with the activity of an MG53 polypeptide or MG53 receptor polypeptide. In certain embodiments, the modulating agents include, for example, a bioactive agent, phosphotidylserine; zinc, for example, in the form of a zinc salt, zinc carrier or zinc conjugate; notoginsing; and an oxidizing agent.

In certain additional aspects the invention relates to compositions and methods related to the treatment and/or prevention of cardiac tissue damage. In certain exemplary embodiments, the invention encompasses, for example, the administration of an effective amount of a therapeutic composition of the invention for the prevention and/or treatment of cell or tissue damage, such as those occurring in subjects suffering from cardiovascular diseases and/or cardiac ischemia/reperfusion injury, hypoxic injury, heart failure, or any combination thereof.

In addition, the invention relates to nucleic acids, including interfering nucleic acids, and polypeptides encoding MG53 and/or MG53 interacting proteins, for example, CSN6, kinesin, caveolin-3 (SEQ ID NO. 8), periaxin, phosphoinositide-3 kinase (PI3K), and myelin-basic-protein, mutants, truncations, fragments, homologs, pseudopeptides and peptidomimetics, as well as compounds that can modulate their activity or their intermolecular interactions with MG53. Therefore, in additional aspects, the present invention encompasses methods for the targeting of caveolin-3 (SEQ ID NO. 8) gene expression, activity, and/or intermolecular interactions for the treatment and/or prevention of a disease or disorder in a subject, for example, cardiovascular diseases and/or cardiac ischemia/reperfusion injury, hypoxic injury, heart failure, or any combination thereof.

In additional aspects, the invention relates to methods of screening and identifying agents useful as therapeutics for the treatment or prevention of cardiovascular diseases and/or cardiac ischemia/reperfusion injury, hypoxic injury, heart failure, or any combination thereof. In certain aspects, the invention encompasses agents, peptides, nucleic acids, or chemical compounds that are agonists of the expression and/or activity and/or phosphorylation of at least one of MG53, CaV3, PI3K, Akt, GSK3•, and/or ERK 1/2. In another aspect, the invention encompasses agents, peptides, nucleic acids, or chemical compounds that are antagonists of the expression or activity, for example, by phosphorylation, of the mitochondrial permeability transition pore (MPTP).

In still additional aspects, the invention relates to methods of treating and/or preventing cardiovascular diseases and/or cardiac ischemia/reperfusion injury, hypoxic injury, heart failure, or any combination thereof, comprising the administration of an effective amount of an agonist of the expression and/or activity of at least one of MG53, CaV3, PI3K, Akt, GSK3•, and/or ERK 1/2.

The preceding general areas of utility are given by way of example only and are not intended to be limiting on the scope of the present disclosure and appended claims. Additional objects and advantages of the present invention will be appreciated by one of ordinary skill in the art in light of the instant claims, description, and examples. For example, the various aspects and embodiments of the invention may be utilized in numerous combinations, all of which are expressly contemplated by the present description. These additional objects and advantages are expressly included within the scope of the present invention.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention.

FIG. 1: MG53 is a muscle specific member of the TRIM protein family. An alignment of the protein sequence of MG53 from various organisms (See SEQ ID NOs.: 1, 3, 5, 9-16) reveals this protein to be a member of the TRIM family. Functional domains are boxed in grey while arrows indicate the domain continues onto another line of the sequence. Boxed Leucine residues indicate the location of a highly conserved Leucine zipper motif.

FIG. 2: Illustrates an exemplary domain comparison of some homologous proteins that contain one or more of the conserved tripartite motifs which are present in MG53. MG53 is unique in it's ability to translocate to an injury site at the cell membrane following multiple forms of insult and mediate repair of the damaged membrane—a function which is not exhibited by the other TRIM family proteins listed. While these TRIM proteins all contain similar domains and/or are expressed in striated muscle, none fully recapitulate the domain organization of MG53.

FIG. 3: MG53 contains unique TRIM and SPRY motifs and is predominantly expressed in muscle cells. A. Diagram of motif structure of MG53. From the results of cDNA cloning and homology searches, several motif sequences are detected in MG53 as shown. The sequences of rabbit and mouse MG53 cDNAs have been deposited in the databases under accession numbers AB231473 and AB231474, respectively. B. Western blot analysis shows the specific expression of MG53 in skeletal and cardiac muscles. Lysate (20 μg total protein per lane) from mouse tissues (lung, kidney, skeletal muscle, liver, heart, brain) were analyzed using anti-mouse MG53 polyclonal antibody. C. Immunofluorescence staining of longitudinal transverse sections from mouse skeletal muscle cells. Scale bar is 125 μM.

FIG. 4: MG53 knockout mice are susceptible to cardiac damage. Paraffin-embedded sections of myocardium from unexercised wild type mice show normal morphology (left) and no Evans blue staining (right). In contrast, and mg53−/− mice display a Evans blue infiltration into myocytes, indicating that there are significant defects in membrane integrity in the mg53−/− heart.

FIG. 5: Loss of MG53 increases susceptibility to cardiac ischemia reperfusion injury. Hearts from wild type (WT) and mg53−/− mice were isolated and perfused on a Langendorff apparatus. Global ischemia was induced for 30 minutes by cessation of perfusate flow. The damage produced in the heart following restoration of perfusate flow (time 0) was measured by enzymatic assays for (a) creatine kinase (CK) or (b) lactate dehydrogenase (LDH). Hearts from mg53−/− mice (dashed lines) show more damage than WT (solid lines). Data is presented as mean±S.D. for each listed time point.

FIG. 6: Functional interaction between MG53 and caveolin-3 regulates dynamic membrane budding process in skeletal muscle. A. Western blot analysis of the expression level of MG53 (upper panel), caveolin-3 (middle panel) and caveolin-1 (lower panel) during C2C12 cell differentiation at the indicated time following induction of differentiation (day 0, 2, 5, 8, 10). B. Whole cell lysate from mouse gastrocnemius skeletal muscle was subjected to co-IP with anti-MG53 (rabbit polyclonal antibody), anti-caveolin-3 (mouse monoclonal antibody), normal rabbit IgG as a negative control and cell lysate as a positive control. C. Confocal images to illustrate the disappearance of filapodia-like structures during the process of C2C12 myotube formation (right panel) compared to myoblasts (left panel). Notice that intracellular vesicles positive for GFP-MG53 are still present in transfected C2C12 myotubes. D. Overexpression of caveolin-3 in C2C12 myoblast cells prevents MG53-induced filapodia-like structures from forming. CHO cells (upper panel) or C2C12 myoblast cells (lower panel) were co-transfected with pcDNA-Cav-3 and GFP-MG53 (10:1) (right panel), or co-transfected with pcDNA vector and GFP-MG53 (10:1) as control (left panel). Confocal images were taken at 48 hours after transfection. Scale bar is 10 μm. E and F. Statistical analysis for C and D. The ratio of cells displaying filapodia-like structures to all green cells was defined as the filapodia-like structure percentage. Data are represented as mean with SEM. (*p<0.01 by t test).

FIG. 7: shRNA-mediated suppression of caveolin-3 expression affects the myotube formation. A. The down-regulation level of caveolin-3 was analyzed by Western blot after transfection with shRNA plasmid for caveolin-3 in C2C12 myotubes (6 days after differentiation). Cells transfected with the scrambled shRNA plasmid acted as a control. B. Down-regulation of caveolin-3 (right panel) by shRNA inhibits myotube formation compared to the control shRNA (left panel). Red fluorescence indicates the transfected cells. Fluorescence microscopy images were taken at 6 days after differentiation induction. Scale bar is 20 μm C. Statistical analysis shows that down-regulation of caveolin-3 significantly inhibits myotube formation at 6 days (*p<0.001 by t test) compared to the control. The ratio of red fluorescent myotubes to all red fluorescent cells served as the percentage of myotubes. Data are represented as mean with SEM. D. Confocal images of C2C12 myoblasts with co-expression of both GFP-MG53 and shRNA for caveolin-3 (right panel) reveal no affect on the filapodia-like structures induced by GFP-MG53 or on the distribution of GFP-MG53 compared to the control shRNA (left panel). Scale bar is 5 μm.

FIG. 8: MG53 knockout mice are susceptible to cardiac damage. Paraffin-embeded sections of myocardium from unexercised wild type mice show normal morphology (left) and no Evans blue staining (right). In contrast, and mg53−/− mice display a Evans blue infiltration into myocytes, indicating that there are significant defects in membrane integrity in the mg53−/− heart.

FIG. 9: Recombinant human TAT-MG53 (See HIV-1 TAT protein, SEQ ID NO. 17) can penetrate cells of different origins. HL-1 cardiomyocytes and 3T3 fibroblasts were incubated with 4 or 8 μg/mL recombinant human TAT-MG53 for 15 minutes at 37° C. Cells were washed three times in a buffered salt solution and then lysed for western blot analysis. Western blot shows that control cells (control) do not contain endogenous MG53, however those incubated with TAT-MG53 contain ample intracellular TAT-MG53. Note that TAT-MG53 is slightly larger than MG53 visualized from skeletal muscle extract (muscle) due to the addition of the TAT cell penetrating peptide to the protein.

FIG. 10: MG53 knockout hearts are vulnerable to IR injury and resistant to IPC protection. (a) Representative immunoblot of MG53 protein levels in myocardial lysates from wt and mg53−/− mice. (b) Hematoxylin and eosinH&E staining of coronal sections of hearts from wt and mg53−/− mice. (c) Change of LDH concentration in the efflux of perfused hearts from wt and mg53−/− mice subjected to 30 min ischemia and various periods of reperfusion with or without IPC (n=8; * p<0.05 vs all of the other three groups; † p<0.05 vs wt IR and wt IPC+IR). (d) Representative photographs and statistical data of infarct size expressed as the percentage of infarct size and total area in perfused wt and mg53−/− mouse hearts subjected to 30 min ischemia and 2 h reperfusion in the presence or absence of IPC (n=8; * p<0.05 vs all of the other three groups; † vs wt IR). (e) Representative examples and statistical data of TUNEL staining of myocardial sections from perfused hearts of wt and mg53−/− mice subjected to 30 min ischemia and 2 h reperfusion (n=8; * p<0.05 vs all of the other three groups; † p<0.05 vs wt IR).

FIG. 11: Overexpression of MG53 protects cardiomyocytes against hypoxia-induced cell death, whereas MG53 gene silencing acerbates cell death. (a) MG53 mRNA expression levels in cardiac tissues in the ischemic area from rats subjected to 45 min ischemia and 12 h reperfusion with or without IPC (n=8; * p<0.05 vs IR and sham; † p<0.05 vs sham). (b) Representative immunoblots and average data of MG53 protein levels in myocardial tissues of the ischemic area from rats subjected to 45 min ischemia and 24 h reperfusion with or without IPC (n=9 for each group; * p<0.05 vs sham and IPC+IR). (c) Representative immunoblots of MG53 protein levels in neonatal cardiomyocytes subjected to hypoxia for various times (n=6 for each time point). (d) Quantitative analysis of cell viability indexed by cellular ATP content in cultured neonatal cardiomyocytes subjected to hypoxia (6-24 h) (n=12 independent experiments; * p<0.05 vs 0 h). (e) Representative blots of MG53 and GFP-MG53 protein levels in lysates of neonatal cardiomyocytes infected with Adv-GFP and Adv-MG53 at indicated titers for 24 h. Similar results were obtained from 5 independent experiments. (f) DNA fragmentation assayed by DNA laddering in cultured neonatal cardiomyocytes subjected to hypoxia (12 h), in the presence or absence of infection with Adv-GFP or Adv-GFP-MG53. Similar results were obtained in another 5 experiments. (g) Representative blots of MG53 protein in lysates of neonatal cardiomyocytes infected with Adv-MG53 or an adenovirus expressing MG53-shRNA or a scramble-shRNA. (h) Cell viability of neonatal cardiomyocytes in the presence or absence of Adv-MG53, MG53-shRNA or Scramble-shRNA, assayed by ATP content (n=12, * p<0.05 as indicated).

FIG. 12: MG53 is essential for activation of the cell survival Akt-GSK3• signaling axis. Representative immunoblots and statistical data of phosphorylated and total Akt and GSK3•• in lysates from cultured neonatal cardiomyocytes in the presence or absence of infection with Adv-GFP or Adv-GFP-MG53 (n=9 for each panel; * p<0.05 vs control and GFP groups). (b) Representative immunoblots and statistical data of phosphorylated and total Akt and GSK3• in perfused wt and mg53−/− mouse hearts with or without IPC (n=8 for each group; * p<0.05 vs all of the other three groups; † p<0.05 vs the two wt groups). (c) Statistical data of infarct size expressed as the percentage of infarcted area of the total area (upper) and LDH release (lower) of perfused wt mouse hearts subjected to 30 min ischemia and 2 h reperfusion with or without LY294002 (5 μM) treatment 10 min before IR or IPC+IR (n=8 for each group, * p<0.05 vs all the other groups). (d) Cell viability assayed by cellular ATP content in neonatal cardiomyocytes infected with Adv-GFP or Adv-GFP-MG53 with or without 1 h pretreatment with LY294002 (10•M), wortmannin (1•M) and Akt inhibitor (1•M). (n=15; * p<0.05 as indicated).

FIG. 13: Co-localization and co-immunoprecipitation of myocardial MG53 with CaV3. (a) Confocal immunofluorescence containing to visualize MG53 (red), CaV3 (green) and nuclei (DAPI; blue) in adult cardiomyocytes (Scale bar is 10 μM). (b) Representative blot of lysates of wt (upper) and mg53−/− hearts (lower) for the co-immunoprecipitation of the p85 subunit of PI3K and CaV3. Similar results were reproduced in 6 independent experiments for both wt and mg53−/− hearts. (c) Representative blot of CaV3 and •-actin in the lysates of neonatal cardiomyocytes infected with Adv-scramble-shRNA or Adv-CaV3-shRNA. This was repeated in 5 independent experiments. (d) Cell viability of neonatal cardiomyocytes infected with Adv-MG53 and subjected to hypoxia (12 h) in the presence or absence of Adv-CaV3-shRNA or Adv-scramble-shRNA (n=9 for each group; * p<0.05 as indicated). (e) Representative immunoblots and statistical data of phosphorylated and total Akt and GSK3• in the lysates of neonatal cardiomyocytes infected with Adv-GFP or Adv-MG53 (30 m.o.i., 24 h) with or without Adv-CaV3-shRNA or Adv-scramble-shRNA (n=5; * p<0.05 vs all other three groups). (f) Co-staining of CaV3 and the p85 subunit of PI3K in wt and mg53−/− heart with or without IPC. Confocal immunofluorescence imaging to visualize the p85 subunit of PI3K (green), CaV3 (red) and nuclei (DAPI; blue) in heart slices from wt and mg53−/− mice with or without application of IPC (n=8; * p<0.05 vs all of the other three groups; † p<0.05 vs wt con, scale bar is 10 μM).

FIG. 14: Protection by IPC against IR injury in rat hearts. (a) Schematic illustration of the protocol used for rat in vivo ischemia/reperfusion (IR) (45 min ischemia followed by reperfusion) with or without 4 episodes of ischemic preconditioning (IPC, i.e., 10 min ischemia followed by 5 min reperfusion). (b) Serum LDH levels in sham rats or those subjected to 45 min ischemia and 4 h reperfusion with or without IPC (n=8 for each group; * p<0.01 vs sham and IPC+IR). (c) Infarct size expressed as the percentage of infarcted area over the area at risk in rats subjected to 45 min ischemia and 24 h reperfusion with or without IPC (n=8 for each group; * p<0.05 vs IR).

FIG. 15: Overexpression of MG53 protects cardiomyocytes against hypoxia-induced cell-death. Quantitative analysis of cell viability indexed by an ATP assay in neonatal cardiomyocytes infected with Adv-GFP or Adv-MG53 for 24 h and then subjected to hypoxia for 12 h (n=12, * p<0.05 vs control, † p<0.05 vs hypoxia+Adv-GFP).

FIG. 16: Co-immunoprecipitation of endogenous MG53 and CaV3 or PI3K in lysates of wt mouse hearts. Similar results were obtained in 4 independent experiments.

FIG. 17: Recombinant MG53 applied in the extracellular space can recognize sites of sarcolemmal membrane disruption in isolated cardiomyocytes. We discovered that acute injury of the cell membrane leads to exposure of a signal to the extracellular space that can be detected by MG53, allowing recombinant MG53 to repair membrane damage when provided in the extracellular space. (A) Here we isolated recombinant RFP-MG53 fusion protein and applied this protein extract to the external media surrounding cultured C2C12 muscle cells. (B) Cells were penetrated with a microelectrode two times damage the sarcolemmal membrane (circles). Confocal images of RFP-MG53 and brightfield were overlaid and clear, progressive accumulation of FFP-MG53 could be observed at the sites of sarcolemmal membrane damage. We also conducted similar experiments with incubation of RFP-MG53 in the extracellular solution of cardiomyocytes (not shown). These results indicate that recombinant MG53 protein can be applied externally to cells and remain effective at targeting to sites of membrane damage.

FIG. 18: Cardioprotective effects of recombinant MG53 during ischemia/reperfusion injury. (A) Wild type mouse (C57B16/J) hearts were subjected to global ischemia/reperfusion (I/R) injury during Langendorff perfusion. Hearts were perfused with Krebs buffer at a flow rate of 2 mL/min and allowed to equilibrate for 30 min before the Krebs buffer was supplemented with MBP-MG53 (40• g/ml) or equimolar concentration of bovine serum albumin (BSA) as a control. Perfusion flow was ceased 5 minutes after the addition of protein and the heart was maintained in an ischemic state for 30 min. To induce I/R injury, the heart was reperfused for 60 min before the heart was removed from the apparatus and stained using Triphenyltetrazolium chloride (TTC) to indicate infarct area using standard techniques. We show representative images of TTC-stained heart slices from BSA (left) and MBP-MG53 (right) treated hearts. Clearly, application of MG53 to the perfusion solution resulted in reduced infarct size compared to a heart treated with BSA as a control. (B) Heart slices were photographed and then infarct area was analyzed using ImageJ software. Infarct size for MBP-MG53 (n=4) treated hearts was significantly reduced compared to MBP-MBP treated hearts (n=4) when measured by T-test. Data presented as means±SEM with * indicating p<0.05. (C) During perfusion of mouse hearts the creatine kinase (CK) release into the perfusate was measured from effluent collected at different times. When CK levels are tested for the first minute following restoration of perfusion after ischemia we find that pre-incubation of MG53 during ischemia can reduce the amount of CK released into the perfusate (n=4 pairs, **p<0.01 by T-test). This data provides an intriguing possibility that recombinant MG53 could have additional protective effect on ischemia-induced injury to the cardiomyocytes. (D) Change of CK concentration in the perfusate hearts subjected to 30 min ischemia and 1 hour reperfusion that were perfused with BSA (black) or recombinant MBP-MG53 (red) during reperfusion at various periods of reperfusion (n=4 pairs, *p<0.05 by ANOVA). The significant reduction of CK with the application of MG53 provides direct support for the cardioprotective function of MG53 during ischemia-reperfusion. (E) Perfusate samples collected for panel D also displayed increased levels of LDH release, however additional replicants are required to reach statistical significance.

FIG. 19: Recombinant MG53 has cardioprotective capacity when applied after the induction of ischemia/reperfusion injury. In a separate series of Langendorff perfusion experiments using the same conditions described in FIG. 2 we tested if recombinant MG53 would have cardioprotective effects if applied after the initiation of PR injury. In these experiments, MG53 was only added after reperfusion of the heart following 30 minutes of ischemia. During reperfusion of mouse hearts the creatine kinase (CK) release into the perfusate was measured from effluent collected at different times. The chart shows changes in CK concentration in the perfusate from hearts subjected to 30 min ischemia and 1 hour reperfusion that were provided either BSA (black) or recombinant MBP-MG53 (red) during reperfusion. (n=4 pairs, *p<0.05 by ANOVA). The significant reduction of CK with the application of MG53 shows that MG53 can be cardioprotective even when applied after the initiation of I/R injury.

FIG. 20: Recombinant MG53 localizes to sites of membrane disruption in whole hearts following I/R injury. Following the I/R injury protocol outlined in FIG. 2, mouse hearts that were perfused with MBP-MG53 were additionally perfused with recombinant AnnexinV coupled to a FITC fluorochrome. Annexin-V will bind phosphatidylserine, a phospholipid normally only found on the inner leaflet of the plasma membrane. Thus, in sites where the sarcolemmal membrane of the cardiomyocytes is disrupted the Annexin-V will have access to the phosphatidylserine and be able to bind the injured sites. Following perfusion of AnnexinV-FITC the hearts are fixed in paraformaldehyde, cyrosectioned and then stained by immunohistochemistry to localize MBP-MG53 using an anti-MBP antibody. (A) Examination of AnnexinV-FITC labeling showed focal injury sites in a random pattern of cardiomyocytes throughout the myocardium. (B) Immunostaining for MBP-MG53 shows that many of these injury sites also display staining for MBP-MG53 (as shown in the overlap of these images in panel C), suggesting that recombinant MG53 can locate and patch sites of membrane disruption in vivo to provide cardioprotective effects against I/R injury. These results show that the same phenomena seen in FIG. 1 in isolated cardiomyocytes also occurs in the whole heart and can provide protective effects for the targeted cardiomyocytes.

FIG. 21: Pharmacokinetics of recombinant MG53 in rat serum following IV injection. (A) Sandwich ELISA analysis for MBP-MG53 levels in rat serum at various timepoints following IV injection. Capture antibodies, 40 ug/ml of mouse monoclonal anti-MG53 (clone # 5257), in 50 ul of PBS are coated on 96 well plate at 37° C. for 1 hr. The antibodies are blocked with 1% BSA in PBS. Treated rat serum, 1:100 dilution in blocking solution, are incubated in each well at 37° C. for 1 hr. The bound MBP-MG53 in serum are detected with HRP conjugated anti-MBP antibodies (1:2,000 dilution, BioLab), OPD reagents (Thermo) are measured at OD488. Control purified MBP-MG53 (0, 7.5, 15, 30, 60, 120, and 240 ng/ml) are used for normalization. Data is presented as mean±SEM for 3 rats. (B) Immunoblot analysis for MBP-MG53 in the same rat serum samples. For each sample, 10 ug of total serum were loaded into individual wells and run on SDS-PAGE gels. Transferred membranes were blotted with anti-MBP antibody for 1 hour at room temperature (top). A parallel loaded gel was stained with Coomassie blue as a control for protein loading (bottom).

FIG. 22: Antibodies generated by four week intertracheal (I.T.) application of MG53 do not block function of MG53. Most protein therapeutics produce some immune response in the course of their use as a therapeutic. To test if recombinant MG53 produces an antibody response and if these antibodies block MG53 function rats were treated with IT application of recombinant MG53 daily for 4 weeks. (A) Equal quantities of recombinant MBPMG53 protein was transferred on Western blot membranes that were then cut into strips. Each strip was incubated with rat serum from a 4 week I.T. application trial as a primary antibody. These serum samples were either pooled (left) or for individual rats (right). Rats that were injected with MBP-MG53 (right) generated an antibody response to the recombinant MBP-MG53 while those injected with saline (left). Final well was a strip reacted with anti-MBP antibody to act as a positive control. Lines with numbers indicate position of molecular weight markers. (B) IgG fractions were isolated from the pooled rat samples (1-10) and then tested by ELISA assay coated with either MBP-MG53 or His-MG53. Isolated IgG antibodies from these rats can recognize MG53. (C) Isolated IgG fraction tested in panel B were tested in an LDH release assay following glass-bead injury of HEK293 cells. Antibodies generated by I.T. application of MBP-MG53 cannot block the activity of MBP-MG53 in resealing the plasma membrane following injury.

FIG. 23: Untagged recombinant MG53 protein is stable and effective at prevention of muscle cell membrane damage. A part of our continuing developmental effort towards the use of recombinant MG53 protein in treatment of damage to striated muscle tissues we have produced large quantities of untagged MG53 (MG53) by removing the maltose binding protein (MBP) immunoaffinity tag from the protein expressed in E. coli (MBP-MG53). (A) The untagged MG53 can be lyophilized for long term storage and then effectively resuspended in physiological saline solution before use. Use of SDS-PAGE gels stained with Coomassie brilliant blue to visualize the protein shows that is it effectively resuspended in solution and that proteins of the appropriate size can be isolated to greater than 95% purity. Each lane contains 10• g of each protein sample. (B) Dose response curves were used to measure the effectiveness of different protein preparations at preventing cell membrane damage. Release of intracellular lactate dehyrodgenase (LDH) from the cell was used as an index of the amount of damage that occurs to the cells. The untagged recombinant MG53 protein (MG53, closed triangles) proved to be highly effective at increasing cell membrane resealing following damage to HEK293 cells (9.0×10̂5 cells per well of a 96 well dish) from exposure to glass microbeads. MG53 appears to be as effective as MBP-MG53 (circles) at preventing cell damage, if not even more effective. The stability of recombinant MG53 was confirmed by repeating this assay after the resuspendend protein is stored at 4° C. for two weeks (open triangles). No decrease in protein efficacy was observed. n=4, error bars are S.E.M. (C) The efficacy of MG53 protein in vivo was confirmed by intramuscular (IM) co-injection of cardiotoxin (CTX) with different protein preparations into the gastrocnemuis muscles of wild type mice. Serum creatine kinase (CK) levels were recorded 6 hours after injection. MG53 and MBP-MG53 proved to be equally effective at prevention of muscle cell membrane damage. For BSA group: n=5; MBP-MG53 group: n=5; MG53 group: n=7. (*: P<0.05 vs BSA). (D) The gastrocnemius muscles of other wild type mice were used for IM co-injection of CTX, MBP-MG53 and recombinant FITC labeled AnnexinV. Annexin V binds phosphatidylserine (PS) that would be exposed when the cell membrane is disrupted. Co-localization of MG53 by immunostaining (right) and FTIC-AnnexinV (left) by confocal microscopy indicates that MG53 can bind exposed PS on injured cells to locate sites of plasma membrane injury. (E) Other gastrocnemius muscles were co-injected with CTX, MG53 and Evans blue dye. Confocal microscopy of sections immunostained for MBP-MG53 (left) show fibers injured with CTX display MBP-MG53 on the periphery of the muscle fibers. Cells that do not display MBP-MG53 on the membrane also have increased Evans blue dye infiltration (right, arrows). (F) Counting of Evans blue positive muscle fibers in multiple microscopy fields show that MBP-MG53 can prevent Evans Blue dye entry into muscle fibers when co-injected with CTX. **: P<0.01 MBP-MBP vs MBP-MG53.

### DETAILED DESCRIPTION

As described herein, MG53 functions as an important modulator of the protective response of cardiovascular diseases and/or cardiac ischemia/reperfusion (IR) injury, hypoxic injury, heart failure, or any combination thereof, and therefore, the targeting and modulating MG53 gene expression, polypeptide synthesis, activity or protein-protein interactions represent a novel therapeutic intervention for the treatment and/or prevention of, for example, IR injury.

The contents of U.S. patent application Ser. No. 12/307,303 filed Jan. 2, 2009; which claims the benefit of PCT/US2007/015815, filed Jul. 11, 2007; which claims the benefit of U.S. Provisional Applications Nos. 60/830,013 filed Jul. 11, 2006; and 60/876,871 filed Dec. 22, 2006; and U.S. patent application Ser. No. 12/328,646 filed Dec. 4, 2008; which claims the benefit of U.S. Provisional Application 61/005,410 filed Dec. 4, 2007; are all incorporated by reference herein in their entirety for all purposes. In addition, the present specification incorporates herein by reference WO 2008/054561; Cai et al., MG53 nucleates assembly of cell membrane repair machinery. Nature Cell Biol., 11(1): p_-_ (January 2009); and Cai et al., MG53 regulates membrane budding and exocytosis in muscle cells. Journal of Biological Chemistry., published online Nov. 24, 2008, in their entirety for all purposes.

The invention is related, in part, to the surprising and unexpected discovery of recombinant nucleic acid sequences and related polypeptides (See, SEQ ID NOs.: 1-15), which are capable of facilitating the treatment and/or protection of cardiac (i.e., myocardial) cells and cardiac tissue from cardiovascular diseases, cardiac ischemia/reperfusion injury, hypoxic injury, heart failure, and/or any combination thereof. Previously, the inventors discovered that vesicular fusion during acute membrane repair is driven by mitsugumin53 (MG53) (SEQ ID NOs. 1-15), a novel muscle-specific tri-partite motif (TRIM) family protein. MG53 expression facilitates, inter alia, intracellular vesicle trafficking to and fusion with the plasma membrane. Previous experimental results also indicated that MG53 function is important in cardiac function and refraction to ischemic/reperfusion and/or hypoxic insult.

Heretofore, interventional approaches against ischemia/reperfusion (IR) injury have centered on the study of ischemic preconditioning (IPC), where transient ischemic events that precede a severe IR episode can reduce damage to the myocardium^{2,10}, as well as in other tissues such as brain, liver, and kidney¹¹⁻¹³. A variety of signaling molecules, including survival kinases (PI3K, Akt and GSK3β) and scaffolding proteins such as calveolin-3 (CaV3, a muscle specific calveolin), have been implicated in IPC. One hypothesis is that ischemic stress simultaneously initiates multiple signaling pathways that temporally and spatially organize in discrete microdomains such as caveolae, lipid-enriched invaginations of the plasma membrane. Calveolins, for example, could function as scaffolds recruiting multiple signaling proteins such as PI3K, ERK 1/2, Src kinases, PKC, eNOS, G-protein-coupled receptors and G proteins, facilitating their activation, thereby providing temporal and spatial regulation of cellular signal transduction. Indeed, disruption of caveolae or its structure protein, CaV3, renders the heart resistant to IPC protection (i.e., the protective effects of IPC are inhibited). However, the molecular mechanism(s) responsible for the rapid coupleing of intracellular signaling to plasma membrane repair and for the temporal and spatial organization of the simultaneously activated IPC signaling events was previously unknown.

Surprisingly and unexpectedly, we have discovered that mitsugumin 53 (MG53), a muscle-specific TRIM-family protein (TRIM72), forms a functional complex with CaV3 in skeletal muscle, and contributes to intracellular vesicle trafficking, membrane fusion, exocytosis, vesicle budding, and myogenesis of striated muscle cells. It is noteworthy that MG53 is exclusively expressed in the heart and skeletal muscle, with highest expression present in myocardium. While our studies established that MG53 functions in repair of acute damage to sarcolemmal membrane in skeletal muscle⁷, they also suggested the mechanism for MG53-mediated cardiac protective effects in response to various insults, particularly ischemic injury. Prior to our discoveries, the functional role of MG53 in the heart was unknown and could not have been reasonably predicted.

Presently, additional evidence is disclosed that demonstrates that MG53 is a crucial component of cardiac IPC machinery. Surprisingly, we have discovered that IR leads to a marked downregulation of MG53 at mRNA and protein levels which is prevented by IPC, and that MG53 deficiency renders the heart more vulnerable to IR-induced cardiac damage, and resistant to IPC protection. In sharp contrast, we have discovered that overexpression of MG53 protects cardiomyocytes against hypoxia/ischemia stress-induced cell injury and cell death. In addition, it has been further discovered that unexpectedly, the intermolecular interaction of MG53 with CaVs is obligated to IPC-mediated activation of cell survival signaling such as PI3K-Akt-GSK3B and ERK 1/2 signaling pathways and resultant cardioprotection. The present findings define MG53 as a primary component of cardiac IPC machinery, marking MG53, and its associated signaling pathways, and interacting proteins as a novel and useful therapeutic targets for the treatment and/or prevention of cardiovascular diseases, cardiac ischemia/reperfusion injury, hypoxic injury, heart failure, and/or any combination thereof.

The biopolymer compositions encompassed by the invention are collectively and interchangeably referred to herein as “MG53 nucleic acids” or “MG53 polynucleotides” or “nucleic acids encoding polypeptides facilitating ischemia/reperfusion and/or hypoxic protection” or and the corresponding encoded polypeptides are referred to as “MG53 polypeptides” or “MG53 proteins” or “polypeptides facilitating ischemia/reperfusion and/or hypoxic protection.” Unless indicated otherwise, “MG53” is used generally to refer to any MG53 related and/or MG53-derived biopolymers as explicitly, implicitly, or inherently described herein.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In the case of conflict, the present specification, including definitions, will control. In addition, the materials, methods, and examples are illustrative only and not intended to be limiting.

In response to external damage and internal degeneration, the cells of the body must repair the membrane surrounding the each individual cell in order to maintain their function and the health of the organism. Defects in the ability of the cell to repair external membranes or be refractory to oxidative stress have been linked to many diseases, such as neurodegenerative diseases (Parkinson's Disease), heart attacks, ischemia, hypoxia, heart failure and muscular dystrophy. In addition, the muscle weakness and atrophy associated with various diseases, as well as the normal aging process, has been linked to altered membrane repair and/or oxidative stress. Moreover, membrane damage and oxidative stress occurs in many other pathologic states outside of chronic disease, for example, UV exposure, minor cuts, dermal abrasion, surgical incisions and ulcers, ischemia, reperfusion, hypoxia in both diabetic and otherwise healthy patients all involve some component of damage to cellular membranes and oxidative stress. In order for these cells to repair their membranes in response to acute damage they make use of small packets of membrane that are inside of the cell, referred to as vesicles. These vesicles are normally found within the cell, but upon damage to the cell membrane, these vesicles move to the damage site and form a patch to maintain the cell integrity. Without this essential function, the cell can die and the cumulative effect of this cellular injury can eventually result in dysfunction of the tissue or organ. It is contemplated that the present invention provides compositions and methods for treating and/or preventing the detrimental effects of cell/tissue damage, in particular, cardiovascular diseases, cardiac ischemia/reperfusion injury, hypoxic injury, heart failure, and/or any combination thereof.

As described above, in certain aspects the present invention relates to MG53 nucleic acids, and MG53 polypeptides encoded from nucleic acids of the invention, which, alone or in combination with other components, can modulate the process of cell membrane repair and protection from cardiovascular diseases, cardiac ischemia/reperfusion injury, hypoxic injury, heart failure, and/or any combination thereof, and the oxidative stress that can occur as a result, in a broad range of cell and tissue types. In certain embodiments, the invention encompasses compositions comprising, for example, MG53 polypeptides, MG53 nucleic acids encoding recombinant MG53 polypeptides; as well as vectors, and host cells comprising the same; antibodies, pseudopeptides, fusion proteins, chemical compounds, and methods for producing the same.

In certain aspects, the present invention also relates to compositions useful as therapeutics for treating and/or prevention of cardiac cell and/or tissue damage due to cardiovascular diseases and/or cardiac ischemia/reperfusion injury, hypoxic injury, heart failure, or any combination thereof. In certain embodiments, this aspect of the invention comprises compositions of the invention together with a pharmaceutically acceptable excipients. Exemplary excipients, which are suitable for use in any embodiment of the invention, are described herein. In certain embodiments, the compositions of the invention may additionally include another biologically active agent that complements or synergizes the activity of the compositions of the invention.

In certain embodiments, the therapeutic compositions of the invention comprise MG53 polypeptides, and nucleic acids encoding MG53 polypeptides, for example, the protein of SEQ ID NO. 1 and MG53 polypeptide mutants, homologs, fragments, truncations, pseudopeptides, peptide analogs, and peptidomimetics (herein, “MG53 polypeptides”), as well as nucleic acids (e.g., small RNAs or antisense RNAs), polypeptides, and compounds that can modulate the activity of MG53 or intermolecular interactions of MG53 with its receptors (i.e., direct or indirect binding or interacting proteins), for example, caveolin-3 (SEQ ID NO. 8), PI3K, Akt, GSK3•, and ERK 1/2.

In additional aspects, the invention includes a composition of the invention in combination with an agent that modulates, synergistically, the activity of an MG53 polypeptide. In certain embodiments, the modulating agents include, for example, phosphotidylserine; zinc, for example, in the form of a zinc salt, zinc carrier or zinc conjugate; notoginsing; or an oxidizing agent.

In certain additional aspects the invention relates to methods for the treatment and/or prevention of cardiovascular diseases and/or cardiac ischemia/reperfusion injury, hypoxic injury, heart failure, or any combination thereof. In certain exemplary embodiments, of this aspect the invention comprises the administration of an effective amount of a therapeutic composition of the invention to an individual, wherein the composition is effective for the prevention and/or treatment of cardiovascular diseases and/or cardiac ischemia/reperfusion injury, hypoxic injury, heart failure, or any combination thereof. In additional aspects, the invention encompasses therapeutic methods further comprising performing an ischemic preconditioning (IPC) step at a time prior to, and/or approximately contemporaneously with, and/or subsequent to the administration of a therapeutic composition of the invention.

In an additional aspect, the invention comprises a method for the treatment and/or prevention of cardiovascular diseases and/or cardiac ischemia/reperfusion injury, hypoxic injury, heart failure, or any combination thereof, comprising the steps of performing ischemic preconditioning on an individual and administering an effective amount of a therapeutic composition of the invention to the individual either prior to the IPC step, substantially contemporaneously, or subsequent to the IPC step. In any embodiment of this aspect of the invention, the method can also include the addition of an agent that modulates the activity or expression of at least one of caveolin-3, PI3K, Akt, GSK3•, and/or ERK 1/2.

In additional aspects, the invention relates to interfering and antisense nucleic acids that modulate the expression of MG53 or an MG53 receptor. “MG53 receptor” includes polypeptides that interact directly and/or indirectly with MG53, and include, for example, caveolin-3 (SEQ ID NO. 8), PI3K, Akt, GSK3•, and/or ERK 1/2, as well as compounds that can modulate their activity or their intermolecular interactions with MG53. Therefore, in additional aspects, the present invention encompasses methods for the targeting of caveolin-3, PI3K, Akt, GSK3•, and/or ERK 1/2, gene expression, activity, and/or intermolecular interactions for the treatment and/or prevention of a disease or disorder in a subject, for example, cardiovascular diseases and/or cardiac ischemia/reperfusion injury, hypoxic injury, heart failure, or any combination thereof.

In additional aspects, the invention encompasses methods of screening and identifying agents from a library of agents useful as therapeutics for the treatment or prevention of cardiovascular diseases and/or cardiac ischemia/reperfusion injury, hypoxic injury, heart failure, or any combination thereof. In certain embodiments of this aspect, the invention encompasses providing a library of chemical compounds and screening for binding, modulation of activity, and/or expression of MG53, CaV3, PI3K, Akt, GSK3•, and/or ERK 1/2, wherein the agent represents a candidate useful as a therapeutic or research tool for the treatment/prevention, and/or study of cardiovascular diseases and/or cardiac ischemia/reperfusion injury, hypoxic injury, heart failure, or any combination thereof. Particularly preferred agents identified according to the methods of the invention include those that are highly specific for the target polypeptide, and therefore, will have few “off target” effects. In general, agents having little or no non-specific effects will demonstrate fewer negative side-effects in vivo. In certain embodiments, the agents are peptides, nucleic acids, or chemical compounds that are agonists of the expression, activity, and/or phosphorylation of at least one of MG53, CaV3, PI3K, Akt, GSK3•, or ERK 1/2. In another aspect, the invention encompasses agents are peptides, nucleic acids, or chemical compounds that are antagonists of the expression or activity, for example, by phosphorylation, of the mitochondrial permeability transition pore (MPTP).

In still additional aspects, the invention relates to methods of treating and/or preventing cardiovascular diseases and/or cardiac ischemia/reperfusion injury, hypoxic injury, heart failure, or any combination thereof, comprising the administration of an effective amount of an agonist of the expression and/or activity of at least one of MG53, CaV3, PI3K, Akt, GSK3•, or ERK 1/2 or an antagonist of the expression or activity of the mitochondrial permeability transition pore (MPTP).

In certain aspects, the invention encompasses an isolated or recombinant nucleic acid encoding a polypeptide, which comprises a combination of amino acid and/or peptide components (i.e., structural components or amino acid domains), which when combined together, result in a polypeptide having the activity as described herein. In one embodiment of this aspect of the invention, the components comprise a RING finger zinc-binding domain, a B-box domain, a Leucine zipper coiled-coil domain, a phospholipid binding domain, a redox sensitive amino acid, an E3-ligase domain, and a SPRY domain, wherein the components are covalently joined contiguously in a single polypeptide, and wherein the polypeptide facilitates treatment and/or prevention of cardiovascular diseases and/or cardiac ischemia/reperfusion injury, hypoxic injury, or heart failure. The nucleic acids encoding the respective amino acid or peptide domains can be cloned from any desired parental gene and combined into a single contiguous using standard molecular biological techniques. In additional embodiments, the invention encompasses novel polypeptides formed by expressing genes or cDNA constructs formed by combining nucleotides encoding amino acid or peptide components from other members of the TRIM family, for example (be accession number) TRIM1 (NM_{—}012216, NM_{—}052817); TRIM2 (AF220018); TRIM3 (AF045239); TRIM4 (AF220023); TRIM5 (AF220025); TRIM6 (AF220030); TRIM7 (AF220032); TRIM8 (AF281046); TRIM5 (AF220036); TRIM10 (Y07829); TRIM11 (AF220125); TRIM13 (AF220127, NM_{—}001007278); TRIM14 (NM_{—}014788, NM_{—}033221); TRIM15 (NM_{—}033229); TRIM16 (AF096870); TRIM17 (AF156271); TRIM18 (AF230976, AF230977); TRIM19 (NM_{—}033244, NM_{—}033250, NM_{—}033240, NM_{—}033239, NM_{—}033247, NM_{—}002675, NM_{—}033246, NM_{—}033249, NM_{—}033238); TRIM20 (NM_{—}000243); TRIM21 (NM_{—}003141); TRIM22 (NM_{—}006074); TRIM23 (NM_{—}033227, NM_{—}001656, NM_{—}033228); TRIM24 (NM_{—}003852, NM_{—}015905); TRIM25 (NM_{—}005082), TRIM26 (NM_{—}003449); TRIM27 (AF230394, AF230393); TRIM28 (NM_{—}005762); TRIM29 (AF230388); TRIM31 (AF230386); TRIM32 (NM_{—}012210); TRIM33 (AF220136); TRIM34 (NM_{—}130390, NM_{—}001003827, NM_{—}130389, NM_{—}001003819); TRIM35 (AB029021); TRIM36 (AJ272269); TRIM37 (AB020705); TRIM38 (U90547); TRIM39 (NM_{—}021253, NM_{—}172016); TRIM40 (AF489517); TRIM41 (NM_{—}033549, NM_{—}201627); TRIM42 (AF521868); TRIM43 (NM_{—}138800); TRIM44 (NM_{—}017583); TRIM45 (NM_{—}025188); TRIM46 (NM_{—}025058); TRIM47 (AY026763); TRIM 48 (AF521869); TRIM49 (NM_{—}020358); TRIM50 (AY081948); TRIM51 (NM_{—}032681); TRIM52 (NM_{—}032765); TRIM53 (XR_{—}016180); TRIM54 (NM_{—}032546, NM_{—}187841); TRIM55 (NM_{—}184087, NM_{—}184085, NM_{—}184086, NM_{—}033058); TRIM56 (NM_{—}030961); TRIM57 (i.e., TRIM59); TRIM58 (NM_{—}015431); TRIM59 (NM_{—}173084); TRIM60 (NM_{—}152620); TRIM61 (XM_{—}373038); TRIM62 (NM_{—}018207); TRIM63 (NM_{—}032588); TRIM64 (XM_{—}061890); TRIM65 (NM_{—}173547); TRIM66 (XM_{—}001716253); TRIM67 (NM_{—}001004342); TRIM68 (NM_{—}018073); TRIM69 (AF302088); TRIM70 (DQ232882, NM_{—}001037330); TRIM71 (NM_{—}001039111); TRIM72 (i.e., MG53; NM_{—}001008274); TRIM73 (AF498998); TRIM74 (NM_{—}198853); TRIM75 (XM_{—}939332).

In another embodiment, the invention comprises an isolated or recombinant polypeptide encoded by nucleic acids of the invention, having a RING finger zinc-binding domain, a B-box domain, a Leucine zipper coiled-coil domain, a phospholipid binding domain, a redox sensitive amino acid, an E3-ligase domain, a SPRY domain, and optionally a calcium binding domain, wherein the components are covalently joined contiguously in a single polypeptide, and wherein the polypeptide facilitates treatment and/or prevention of cardiovascular diseases and/or cardiac ischemia/reperfusion injury, hypoxic injury, or heart failure.

The present description highlights the important amino acid structural components or features for creating polypeptides able to facilitate treatment and/or prevention of cardiovascular diseases and/or cardiac ischemia/reperfusion injury, hypoxic injury, or heart failure (i.e., a RING finger zinc-binding domain, a B-box domain, Leucine zipper coiled-coil domain, a phospholipid binding domain, redox sensitive amino acid, E3-ligase domain, SPRY domain). It is important to note that although RING finger zinc-binding domains, a B-box domains, Leucine zipper coiled-coil domains, a phospholipid binding domains, redox sensitive amino acids, E3-ligase domains, SPRY domains, and calcium binding domains may vary between evolutionarily related proteins as well as unrelated proteins, as indicated above, there exists a number of genes belonging to the TRIM family, which includes MG53, which contain one or all of the above structural components or domains. As those of skill in the art would appreciate, these domains may be readily cloned from the gene or cDNA of a TRIM family member, and grafted or cloned into the framework of another TRIM family gene (i.e., MG53) using well known techniques in molecular biology in order to create novel proteins. Also, because it is generally recognized that evolutionarily conserved amino acid sequences will function similarly, it is within the abilities of those skilled in the art to generate additional proteins in accordance with the instant teachings, and to assess the ability of the recombinant proteins to facilitate membrane repair without undue experimentation. As such, recombinant proteins assembled from the domains of the TRIM family members, for example, those identified above, is expressly contemplated as being within the scope of the invention.

In another embodiment, the invention encompasses an isolated or recombinant nucleic acid encoding an MG53 polypeptide as set forth in SEQ ID NOs.: 1, 3, 5, 7, 8, 9-15, and/or a homolog, or fragment thereof, wherein the polypeptide facilitates treatment and/or prevention of cardiovascular diseases and/or cardiac ischemia/reperfusion injury, hypoxic injury, or heart failure.

In an additional aspect, the invention relates to compositions comprising a polypeptide of the invention in combination with at least one other agent, which is capable of facilitating treatment and/or prevention of cardiovascular diseases and/or cardiac ischemia/reperfusion injury, hypoxic injury, or heart failure. In certain embodiments, the agent acts synergistically, via direct or indirect interaction with the polypeptide of the invention, to facilitate the treatment and/or prevention of cardiovascular diseases and/or cardiac ischemia/reperfusion injury, hypoxic injury, or heart failure. For example, agents such as phosphotidylserine, zinc, oxidizing agents, and plant extracts can modulate the membrane repair activity of the polypeptides of the invention.

Therefore, in additional embodiments, any of the polypeptide-containing compositions encompassed by the invention may also comprise, in combination, an effective amount of at least one of a phospholipid; a zinc containing agent; an oxidizing agent; a plant extract or a combination thereof. In certain embodiments the phospholipid is phosphytidylserine. In additional embodiments, the zinc containing agent is a zinc ionophore, for example, Zn-1-hydroxypyridine-2-thine (Zn-HPT). In other embodiments, the oxidizing agent is thimerosal. In additional embodiments, the plant extract is notoginsing extract.

In certain additional apects, the invention relates to a composition comprising an isolated or recombinant polypeptide of the invention in combination with a pharmaceutically acceptable carrier. In additional embodiments, the composition may further comprise, in combination, an effective amount of at least one of a phospholipid; a zinc containing agent; an oxidizing agent; a plant extract or a combination thereof. In certain embodiments the phospholipid is phosphytidylserine. In additional embodiments, the zinc containing agent is a zinc ionophore, for example, Zn-1-hydroxypyridine-2-thine (Zn-HPT). In other embodiments, the oxidizing agent is thimerosal. In additional embodiments, the plant extract is notoginsing extract.

The present invention also relates to the surprising and unexpected finding that polypeptides of the invention can treat and/or prevent ischemic/reperfusion and/or hypoxic injury to myocardial cells and/or tissue. Without being bound by any particular theory, it is believed that the repair mechanism is mediated by the oxidative-induced formation of polypeptide oligomers, e.g., dimers, through the coiled-coil domain in the protein, which contains a leucine zipper protein-protein interaction motif.

The current results indicate that the activity of polypeptides of the invention, for example, MG53, is primarily induced by entry of the oxidative extracellular milieu into the reduced environment in the cellular compartment. This mechanism allows for the polypeptides to act as a sensor of cellular redox state and oligomerize to form homologous complexes at the plasma membrane by interaction with specific lipid components of the cell membrane. As described in a prior application, zinc (Zn) is required for MG53-mediated membrane resealing, and the presence of additional Zn can improve the activity of MG53; an extract from the plant notoginsing can also improve the function of MG53 in membrane resealing; and MG53 requires its endogenous E3-ligase activity to produce membrane repair following acute damage. Thus, it is likely that one or more of these activities is also important for mediating the treatment and/or prevention of ischemic/reperfusion and/or hypoxic injury to myocardial cells and/or tissue.

Additional aspects of the invention related to the surprising discovery that extracellular application or administration of polypeptides of the invention is also efficacious. Polypeptides suitable for extracellular administration include, for example, MG53, MG53 fusion proteins, for example, MG53 fused with cell penetrating peptides, for example, HIV-TAT protein (See WO 2008/054561, which is incorporated herein by reference). As such, certain embodiments of this aspect comprise therapeutic compositions comprising polypeptides of the invention, for example, MG53, in combination with a pharmaceutically acceptable carrier, wherein the therapeutic composition is administered systemically, and wherein the systemically administered composition is effective in facilitating treatment and/or prevention of cardiovascular diseases and/or cardiac ischemia/reperfusion injury, hypoxic injury, or heart failure.

In certain additional embodiments, the therapeutic compositions of the invention further comprise, in combination with a polypeptide of the invention, one or more additional ingredients, including a phospholipid; a zinc containing agent; an oxidizing agent; a plant extract or a combination thereof, which have a synergistic effect on the activity of the polypeptides of the invention. In additional embodiments, the therapeutic of the invention may comprise one or more biologically active ingredients such as, Analgesics, Antacids, Antianxiety Drugs, Antiarrhythmics, Antibacterials, Antibiotics, Anticoagulants and Thrombolytics, Anticonvulsants, Antidepressants, Antidiarrheals, Antiemetics, Antifungals, Antihistamines, Antihypertensives, Anti-Inflammatories, Antineoplastics, Antipsychotics, Antipyretics, Antivirals, Barbiturates, Beta-Blockers, Bronchodilators, Cold Cures, Corticosteroids, Cough Suppressants, Cytotoxics, Decongestants, Diuretics, Expectorants, Hormones, Hypoglycemics (Oral), Immunosuppressives, Laxatives, Muscle Relaxants, Sedatives, Sex Hormones, Sleeping Drugs, Tranquilizer, Vitamins or a combination thereof.

In additional aspects, the invention relates to methods of treating or preventing cardiovascular diseases and/or cardiac ischemia/reperfusion injury, hypoxic injury, or heart failure comprising the steps of administering to an individual an effective amount of a nucleic acid encoding a polypeptide of the invention, for example, MG53, homologs, fragments, and derivatives thereof, wherein the polypeptide is effective for treating or prevention ischemia/reperfusion and/or hypoxic injury of myocardial cells or tissue in vitro, in vivo or ex vivo. In an additional aspect, the invention relates to methods of treating and/or preventing a disease or pathological condition related to cardiovascular diseases and/or cardiac ischemia/reperfusion injury, hypoxic injury, or heart failure comprising administering to an individual an effective amount of a composition comprising a nucleic acid encoding a polypeptide of the invention, for example, MG53, homolog, fragment or derivative thereof, in combination with a pharmaceutically acceptable carrier, wherein the composition is effective in treating and/or preventing cell myocardial cell or tissue damage. In certain embodiments, the disease or pathological condition related to cell or tissue damage includes muscular dystrophy, cardiac ischemia, heart failure, aging degeneration, or any combination thereof.

In any of the methods described herein, the nucleic acids or polypeptides of the invention may be delivered or administered in any pharmaceutically acceptable form, and in any pharmaceutically acceptable route as described in further detail below. For example, compositions comprising nucleic acids and/or polypeptides of the invention can be delivered systemically or administered directly to a cell or tissue for the treatment and/or prevention of myocardial cell or tissue damage. In certain additional embodiments, the nucleic acids and/or polypeptides of the invention comprise a carrier moiety that improves bioavailability, drug half-life, efficacy or potency.

In an additional aspect, the invention relates to an isolated or recombinant membrane repair polypeptide complex. As presented in detail below, MG53 polypeptides demonstrate the ability to interact (e.g., bind non-covalently) and form complexes, either directly or indirectly, with a number other cellular proteins, in particular, CaV3, PI3K, Akt, GSK3•, and ERK 1/2. In an embodiment of this aspect, the invention comprises a recombinant chimeric nucleic acid comprising, in a single open reading frame, a polynucleotide encoding an MG53 polypeptide linked in a contiguous nucleic acid to another polynucleotide encoding another polypeptide, for example, CaV3. In additional embodiments, the chimera may comprise a plurality of polynucleotides encoding any combination of MG53, CaV3, PI3K, Akt, GSK3•, and ERK 1/2 linked in a single contiguous nucleic acid, which is comprised within a single open reading frame. The translation results in a single polypeptide containing functional domains of one or more of MG53, CaV3, PI3K, Akt, GSK3•, and ERK 1/2, wherein the chimeric protein complex is able to facilitate the repair or prevention of myocardial cell or tissue damage due to ischemia/reperfusion injury and/or hypoxic injury. The invention further comprises a method of treating or preventing myocardial cell or tissue damage due to ischemia/reperfusion injury and/or hypoxic injury comprising administering to a cell an effective amount of a nucleic acid encoding a chimeric polypeptide of the invention, wherein the complex is capable of repair or prevention of myocardial cell or tissue damage due to ischemia/reperfusion injury and/or hypoxic injury. In still an additional embodiment, the invention includes a method of treating or preventing disease or pathological condition related to cell or tissue damage comprising administering to an individual an effective amount of isolated chimeric polypeptide of the invention, wherein the chimeric polypeptide complex is capable of ameliorating the effects of the disease or pathological condition.

In additional aspects, the invention relates to methods of treatment or prevention of myocardial cell or tissue damage due to ischemia/reperfusion injury and/or hypoxic injury comprising modulating the expression level or activity or both of at least one of CaV3, PI3K, Akt, GSK3•, or ERK 1/2.

In still additional aspects, the invention relates to methods of screening for compounds that are effective for the treatment or prevention of myocardial cell or tissue damage due to ischemia/reperfusion injury and/or hypoxic injury by contacting at least one of MG53, CaV3, PI3K, Akt, GSK3•, or ERK 1/2, with a test compound; and measuring the binding of the test compound, and/or the activity of MG53, CaV3, PI3K, Akt, GSK3•, or ERK 1/2, and/or the measuring the effects on myocardial cell viability in response to IR or hypoxic insult.

As described in detail below, and as would be readily appreciated by those skilled in the art, the recombinant membrane repair polypeptides can be produced in prokaryotic cells or eukaryotic cells, for example, mammalian cells and then secreted into the extracellular solution through protein engineering, an approach that should produce large quantities of functional protein.

In addition, the invention relates to nucleic acids, including interfering nucleic acids that hybridize to a nucleic acid encoding MG53 CaV3, PI3K, Akt, GSK3•, or ERK 1/2, mutants, truncations, fragments, or homologs thereof, for the treatment or prevention of myocardial cell or tissue damage due to ischemia/reperfusion injury and/or hypoxic injury. In any of the embodiments described herein, therapeutic compositions can be administered in any suitable pharmaceutical form as described herein or as commonly known in the art.

In an aspect, the invention provides an isolated nucleic acid encoding polypeptide molecules, for example, MG53 gene hybridizing nucleic acid molecules, and nucleic acids encoding MG53 polypeptides having at least 30%, 40%, 50%, 60%, 70%, 80%, 90% or 100% identity to the nucleic acids disclosed in SEQ ID NOS: 2, 4, and 6. In certain embodiments, the isolated nucleic acid molecules of the invention will hybridize under stringent conditions to a nucleic acid sequence complementary to a nucleic acid molecule that includes a protein-coding sequence of an MG53 nucleic acid sequence. The invention also includes an isolated nucleic acid that encodes an MG53 polypeptide, or a fragment, homolog, analog, fusion protein, or derivative thereof. For example, the nucleic acid can encode a polypeptide at least 30%, 40%, 50%, 60%, 70%, 80%, 90% or 100% identity to a polypeptide comprising the amino acid sequences of SEQ ID NOS: 1, 3, 5, 7, 8, and 9-15. The nucleic acid can be, for example, a genomic DNA fragment or a cDNA molecule that includes the nucleic acid sequence of any of SEQ ID NOS: 2, 4, and 6.

Also included in the invention is an oligonucleotide, e.g., an oligonucleotide which includes at least 6 contiguous nucleotides of an MG53 nucleic acid (e.g., SEQ ID NOS: 2, 4, and 6) or a complement of said oligonucleotide.

Also included in the invention are substantially purified polypeptides, for example, MG53 polypeptides (SEQ ID NOS: 1, 3, 5, 7, 8, and 9-15). In certain embodiments, the polypeptides, e.g., MG53 polypeptides, include an amino acid sequence that is substantially identical to the amino acid sequence of a human MG53 polypeptide (SEQ ID NO.:1).

In addition, the invention comprise the use of a therapeutic composition comprising an effective amount of an agent that modulates at least one of MG53 activity, MG53 expression, or the MG53 signaling cascade in a cardiac cell in the manufacture of a medicament for the treatment and/or prevention of cardiac injury. The use of the therapeutic composition can comprise an effective amount of from 0.1 mg/kg and 1000 mg/kg body weight/day.

In certain embodiments the therapeutic agent is at least one of an MG53 polypeptide; an MG53 receptor polypeptide; a nucleic acid encoding an MG53 polypeptide; a nucleic acid encoding an MG53 receptor polypeptide; an inhibitory or antisense RNA specific for a nucleic acid encoding MG53, an MG53 receptor, caveolin-3, PI3K, Akt, GSK3., or ERK 1/2; or an agonist or antagonist of MG53, an MG53 receptor, caveolin-3, PI3K, Akt, GSK3., ERK 1/2 or MPTP. In certain embodiments, the agonist of MG53 activity comprises at least one of phosphotidylserine, zinc or zinc salt, Zn-1-hydroxypyridine-2-thine (Zn-HPT), notoginsing, an oxidizing agent, thimerosal, or combination thereof. The polypeptide can have the amino acid sequence of at least one of SEQ ID NOs.: 1, 3, 5, 9, 10, 11, 12, 13, 14, 15, or 16 or bioactive portion thereof.

In still other embodiments, the agent is a stem cell capable of differentiation into a cardiac myocyte, and wherein the stem cell has been modified such that it demonstrates enhanced activity or expression of MG53. The therapeutic composition can further comprise a pharmaceutically acceptable carrier or excipient. In certain embodiments, the cardiac injury comprises cardiac cell or myocardial tissue injury due to at least one of cardiovascular disease, cardiac ischemia/reperfusion injury, hypoxic injury, heart failure, or a combination thereof.

In another embodiment, the invention also includes the use of a therapeutic composition comprising an effective amount of an MG53 polypeptide or MG53 nucleic acid, and a pharmaceutically acceptable excipient, in the manufacture of a medicament for the treatment and/or prevention of cardiac ischemic/reperfusion or hypoxic injury.

The invention also features antibodies that immunoselectively-bind to polypeptides, for example, MG53, polypeptides, or fragments, homologs, analogs, pseudopeptides, peptidomimetics or derivatives thereof.

In another aspect, the invention includes pharmaceutical compositions that include therapeutically- or prophylactically-effective amounts of a therapeutic and a pharmaceutically-acceptable carrier. The therapeutic can be a nucleic acid, e.g., a MG53 nucleic acid, for example, a peptide nucleic acid, a cDNA, or RNA, such as for example, a small inhibitory RNA; a membrane repair polypeptide for example, MG53; or an antibody specific for a MG53 polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes an endogenous or exogenously expressed nucleic acid endocing a membrane repair polypeptide, for example a MG53 nucleic acid, under conditions allowing for expression of the polypeptide encoded by the DNA. If desired, the polypeptide can then be recovered.

In still another aspect, the invention includes a method of producing a polypeptide by culturing a cell that contains an endogenous nucleic acid encoding a polypeptide, for example a MG53 nucleic acid, disposed upstream or downstream of an exogenous promoter. In certain embodiments, the exogenous promoter is incorporated into a host cell's genome through homologous recombination, strand break or mismatch repair mechanisms.

In another aspect, the invention includes a method of detecting the presence of a polypeptide of the invention, for example, an MG53 polypeptide, in a sample. In the method, a sample is contacted with a compound that selectively binds to the polypeptide under conditions allowing for formation of a complex between the polypeptide and the compound. The complex is detected, if present, thereby identifying the membrane repair polypeptide, for example, an MG53 polypeptide, within the sample.

The invention also includes methods to identify specific cell or tissue types based on their expression of a nucleic acid encoding a polypeptide of the invention, for example a MG53 polypeptide or a related fusion polypeptide, thereof. For example, in certain embodiments the invention includes fusion proteins comprising a “tag” or indicator portion and, for example, an MG53 portion. In certain aspects the tag or indicator portion can be a peptide adapted for purification purposes, for example, FLAG tag, 6×His tag, Maltose-Binding Protein (MBP) tag, or the like. In other aspects, the tag peptide comprises a peptide adapted for providing a signal such as an antibody epitope or a fluorescent peptide. Still other aspects include the fusion of the MG53 with a peptide that is adapted for mediating subcellular localization or translocation across a cellular membrane, for example, a TAT fusion protein from the HIV virus To facilitate cell penetration or a modified cellular localization tag to couple MG53 to particular cellular organelles.

Also included in the invention is a method of detecting the presence of a nucleic acid molecule of the invention in a sample by contacting the sample with a nucleic acid probe or primer, and detecting whether the nucleic acid probe or primer bound to a nucleic acid encoding, for example, an MG53 polypeptide.

In a further aspect, the invention provides a method for modulating the activity of a polypeptide of the invention, for example, an MG53 polypeptide, by contacting a cell that includes the MG53 polypeptide, with a compound that binds to the MG53 polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic of the invention in the manufacture of a medicament for treating or preventing disorders or syndromes including, e.g., cardiovascular disease, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, hypercoagulation, ischemia/reperfusion injury, hypoxic injury, oxidative damage, age-related tissue degeneration, surgically related lesions, heart failure, secondary pathologies caused by heart failure and hypertension, hypotension, angina pectoris, myocardial infarction, tuberous sclerosis, heart attacks, heart failure, muscular dystrophy, stroke, and/or other pathologies and disorders of the like.

The therapeutic composition of the invention comprises, in certain embodiments, for example, a nucleic acid encoding a MG53; a nucleic acid that binds a nucleic acid encoding MG53; an MG53 polypeptide, peptide analog, pseudopeptide or peptidomimetic based thereon; a small molecule modulator of MG53 or an MG53 protein-protein interaction; or a MG53-specific antibody or biologically-active derivatives or fragments thereof. As described herein, MG53 mediates the treatment and/or prevention of ischemia/reperfusion injury and/or hypoxic injury of myocardial cells or tissue. Therefore, targeting the expression and/or activity of these nucleic acids, polypeptides, and homologs thereof will allow for a novel treatment of various acute and chronic diseases and conditions related to ischemic damage or cardiac tissue.

In still other embodiments, the invention comprises therapeutic compositions useful as a surgical adjuvant. In any of the embodiments described herein, the surgical adjuvant composition of the invention can be used or applied as a stand alone therapeutic directly to the surgical site or it can be integrally associated with a surgical or medical implement, for example, the therapeutic of the invention may be conjugated to a polymer-based stent, tube or other implantable device, such that the therapeutic diffuses to the site of action in a controlled manner to accelerate healing and/or to minimize trauma from an invasive surgical procedure. In another embodiment, the therapeutic composition of the invention is applied as, for example, a film or coating to the medical implement such that the therapeutic diffuses into the blood stream or surrounding tissues and/or wears away, and is thereby delivered directly to the site of tissue damage; minimizing or ameliorating the amount of damage that occurs due to the use of the surgical implement or procedure.

Furthermore, due to the muscle-specific nature of the expression of the endogenous MG53 gene, the invention encompasses methods for the treatment and/or prevention of any type of muscle or vascular cell/tissue injury, for example, tissue injury that occurs as a result of cardiovascular disease, for example, myocardial infaraction; or rigorous physical activity, for example, sports-related injuries, comprising administering an effective amount of the therapeutic of the invention to a subject in need thereof.

In any aspect of the invention, the therapeutic composition of the invention can be in any pharmaceutically acceptable form and administered by any pharmaceutically acceptable route, for example, the therapeutic composition can be administered as an oral dosage, either single daily dose or unitary dosage form, for the treatment of a muscle damage due to a myocardial infarction, sclerotic lesion, or muscle tear due to sports-related activity to promote the regeneration and repair of the damaged muscle tissue. Such pharmaceutically acceptable carriers and excipients and methods of administration will be readily apparent to those of skill in the art, and include compositions and methods as described in the USP-NF 2008 (United States Pharmacopeia/National Formulary), which is incorporated herein by reference in its entirety.

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The active compounds will generally be formulated for parenteral administration, e.g., formulated for injection via the intravenous, intracellular, intrathecal, intramuscular, sub-cutaneous, intra-lesional, or even intraperitoneal routes. The preparation of an aqueous composition that contains a marker antibody, conjugate, inhibitor or other agent as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

In addition, the invention relates to nucleic acids, including interfering nucleic acids, and polypeptides encoding membrane repair interacting proteins and/or MG53 interacting proteins, and homologs thereof; psuedopeptides and peptidomimetics; as well as compounds that can modulate the activity of membrane repair polypeptides or MG53 or their intermolecular interactions.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. In addition, a cDNA encoding polypeptides of the invention, for example, MG53, CaV3, PI3K, Akt, GSK3•, and ERK 1/2 may be useful in gene therapy when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for predisposition to a disorder or syndrome including, e.g., the diseases and disorders disclosed above and/or other pathologies and disorders of the like. The method includes contacting a test agent to a nucleic acid or polypeptide encoding MG53, CaV3, PI3K, Akt, GSK3•, and/or ERK 1/2, and determining if the test agent binds to said target. Binding of the test agent to a nucleic acid or polypeptide encoding MG53, CaV3, PI3K, Akt, GSK3•, and/or ERK 1/2, indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a nucleic acid of the invention. Expression or activity of a polypeptide of the invention is then measured in the test animal, as is expression or activity of the protein in a control animal which recombinantly-expresses the polypeptide of the invention and is not at increased risk for the disorder or syndrome. Next, the expression of polypeptides of the invention in both the test animal and the control animal is compared. A change in the activity of the polypeptide in the test animal relative to the control animal indicates the test compound is a modulator of latency of the disorder or syndrome.

In yet another aspect, the invention includes a method for determining the presence of or predisposition to a disease associated with dysfunctional or altered levels of a nucleic acid or polypeptide for MG53, CaV3, PI3K, Akt, GSK3•, and/or ERK 1/2, in a subject (e.g., a human subject). The method includes measuring the amount of the a nucleic acid or polypeptide for MG53, CaV3, PI3K, Akt, GSK3•, and/or ERK 1/2, in a test sample from the subject and comparing the amount of the nucleic acid or polypeptide in the test sample to the amount of the nucleic acid or polypeptide present in a control sample. An alteration in the level of the nucleic acid or polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various disorders as well as to determine the stage of particular disorders.

In yet another aspect, the invention can be used in a method to identity the cellular receptors of MG53 and downstream effectors of the invention by any one of a number of techniques commonly employed in the art. These include but are not limited to the two-hybrid system, affinity purification, co-precipitation with antibodies or other specific-interacting molecules.

As used herein, the term “antagonist” or is used generally to refer to an agent capable of direct or indirect inhibition of expression, translation, and/or activity. Also, as used herein “MG53 receptor” relates generally to any protein or fragment thereof capable of undergoing binding to a MG53 protein. In certain aspects, the modulation of MG53 activity is accomplished by, for example, the use of or modulation of, for example, MG53 binding partners, i.e., factors that directly or indirectly bind to MG53, and enhance or neutralize its biological activities, and include, for example, neutralizing anti-MG53 antibodies, caveolin-3, anti-caveolin-3 antibodies, pseudopeptides, peptide analogs or peptidomimetics that bind and disrupt MG53 or CaV3 activity or intermolecular interactions; or the use of nucleotide sequences derived from MG53 or CaV3 genes, including coding, non-coding, and/or regulatory sequences to modulate expression by, for example, antisense, ribozyme, and/or triple helix approaches.

In another aspect, the present invention features a nucleic acid molecule, such as a decoy RNA, dsRNA, siRNA, shRNA, micro RNA, aptamers, antisense nucleic acid molecules, which down regulates expression of a sequence encoding MG53 proteins, and/or MG53 receptors, for example, caveolin-3. In another embodiment, a nucleic acid molecule of the invention has an endonuclease activity or is a component of a nuclease complex, and cleaves an MG53 and/or CaV3 mRNA.

In one embodiment, a nucleic acid molecule of the invention comprises between 12 and 100 bases complementary to RNA having a nucleic acid sequence encoding a member selected from the group of MG53, CaV3, PI3K, Akt, GSK3B, and ERK 1/2. In another embodiment, a nucleic acid molecule of the invention comprises between 14 and 24 bases complementary to RNA having a nucleic acid sequence encoding a member selected from the group of MG53, CaV3, PI3K, Akt, GSK3B, and ERK 1/2. In any embodiment described herein, the nucleic acid molecule can be synthesized chemically according to methods well known in the art.

In another aspect the present invention provides a kit comprising a suitable container, the active agent capable of inhibiting membrane repair polypeptide activity, expression or binding in a pharmaceutically acceptable form disposed therein, and instructions for its use.

In another aspect, the invention relates to a method for diagnosing or monitoring disorder or disease or progression comprising detecting for the presence of a nucleotide polymorphism in the membrane repair gene, for example, MG53 gene, associated with the disease, through the detection of the expression level of a member selected from the group of MG53, CaV3, PI3K, Akt, GSK3B, and ERK 1/2.

Polymorphisms have been identified that correlate with disease severity. (See, Zhong et al., Simultaneous detection of microsatellite repeats and SNPs in the macrophage migration inhibitory factor gene by thin-film biosensor chips and application to rural field studies. Nucleic Acids Res. 2005 Aug. 2; 33(13):e121; Donn et al., A functional promoter haplotype of macrophage migration inhibitory factor is linked and associated with juvenile idiopathic arthritis. Arthritis Rheum. 2004 May; 50(5):1604-10; all of which are incorporated herein by reference in their entirety for all purposes.). “MG53 or MG53 receptor gene” or includes the 5′ UTR, 3′ UTR, promoter sequences, enhancer sequences, intronic and exonic DNA of the gene as well as the mRNA or cDNA sequence.

As one of ordinary skill will comprehend, the MG53 or MG53 receptor gene polymorphisms associated with tissue repair disorders, and hence useful as diagnostic markers according to the methods of the invention may appear in any of the previously named nucleic acid regions. Techniques for the identification and monitoring of polymorphisms are known in the art and are discussed in detail in U.S. Pat. No. 6,905,827 to Wohlgemuth, which is incorporated herein by reference in its entirety for all purposes.

Certain aspects of the invention encompass methods of detecting gene expression or polymorphisms with one or more DNA molecules wherein the one or more DNA molecules has a nucleotide sequence which detects expression of a gene corresponding to the oligonucleotides depicted in the Sequence Listing. In one format, the oligonucleotide detects expression of a gene that is differentially expressed. The gene expression system may be a candidate library, a diagnostic agent, a diagnostic oligonucleotide set or a diagnostic probe set. The DNA molecules may be genomic DNA, RNA, protein nucleic acid (PNA), cDNA or synthetic oligonucleotides. Following the procedures taught herein, one can identify sequences of interest for analyzing gene expression or polymorphisms. Such sequences may be predictive of a disease state.

Diagnostic Oligonucleotides of the Invention

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

In certain aspects, the invention relates to diagnostic oligonucleotides and diagnostic oligonucleotide set(s), for which a correlation exists between the health status of an individual, and the individual's expression of RNA or protein products corresponding to the nucleotide sequence. In some instances, only one oligonucleotide is necessary for such detection. Members of a diagnostic oligonucleotide set may be identified by any means capable of detecting expression or a polymorphism of RNA or protein products, including but not limited to differential expression screening, PCR, RT-PCR, SAGE analysis, high-throughput sequencing, microarrays, liquid or other arrays, protein-based methods (e.g., western blotting, proteomics, mass-spectrometry, and other methods described herein), and data mining methods, as further described herein.

In the context of the invention, nucleic acids and/or proteins are manipulated according to well known molecular biology techniques. Detailed protocols for numerous such procedures are described in, e.g., in Ausubel et al. Current Protocols in Molecular Biology (supplemented through 2000) John Wiley & Sons, New York (“Ausubel”); Sambrook et al. Molecular Cloning-A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”).

The description below of the various aspects and embodiments is provided with reference to the exemplary nucleic acids of the invention. However, the various aspects and embodiments are also directed to genes which encode homologs, orthologs, and paralogs of other membrane repair proteins, membrane repair polypeptide binding proteins, and membrane repair polypeptide receptor genes and include all isoforms, splice variants, and polymorphisms. Those additional genes can be analyzed for target sites using the methods described for MG53 and MG53 receptor proteins, and/or genes. Thus, the inhibition and the effects of such inhibition of the other genes can be performed as described herein.

By “down-regulate” it is meant that the expression of the gene, or level of RNAs or equivalent RNAs encoding one or more proteins, or activity of one or more proteins, is reduced below that observed in the absence of the nucleic acid molecules of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of genes with the nucleic acid molecule of the instant invention is greater in the presence of the nucleic acid molecule than in its absence.

By “up-regulate” is meant that the expression of the gene, or level of RNAs or equivalent RNAs encoding one or more protein subunits, or activity of one or more protein subunits is greater than that observed in the absence of the nucleic acid molecules of the invention. For example, the expression of a gene can be increased in order to treat, prevent, ameliorate, or modulate a pathological condition caused or exacerbated by an absence or low level of gene expression. In one embodiment the invention relates to a method for treating or preventing ischemic reperfusion or hypoxic injury to myocardial tissue by up-regulating the expression, and/or activity of an MG53 and/or MG53 receptor gene.

By “modulate” is meant that the expression of the gene, or level of RNAs or equivalent RNAs encoding one or more proteins, or activity of one or more proteins is up-regulated or down-regulated, such that the expression, level, or activity is greater than or less than that observed in the absence of the nucleic acid molecules of the invention.

By “gene” it is meant a nucleic acid that encodes RNA, for example, nucleic acid sequences including but not limited to a segment encoding a polypeptide.

“Complementarity” refers to the ability of a nucleic acid to form hydrogen bond(s) with another RNA sequence by either traditional Watson-Crick or other non-traditional types.

By “RNA” is meant a molecule comprising at least one ribonucleotide residue. By “ribonucleotide” or “2′-OH” is meant a nucleotide with a hydroxyl group at the 2′ position of a D-ribo-furanose moiety.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra).

By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol, 40, 1-49, which are incorporated herein by reference in their entirety. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

Long double-stranded RNAs (dsRNAs; typically >200 nt) can be used to silence the expression of target genes in a variety of organisms and cell types (e.g., worms, fruit flies, and plants). Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand. In mammalian cells, introduction of long dsRNA (>30 nt) initiates a potent antiviral response, exemplified by nonspecific inhibition of protein synthesis and RNA degradation. The mammalian antiviral response can be bypassed, however, by the introduction or expression of siRNAs.

Injection and transfection of dsRNA into cells and organisms has been the main method of delivery of siRNA. And while the silencing effect lasts for several days and does appear to be transferred to daughter cells, it does eventually diminish. Recently, however, a number of groups have developed expression vectors to continually express siRNAs in transiently and stably transfected mammalian cells. (See, e.g., Brummelkamp T R, Bernards R, and Agami R. (2002). A system for stable expression of short interfering RNAs in mammalian cells. Science 296:550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, which are herein incorporated by reference in their entirety).

By “vectors” is meant any nucleic acid-based technique used to deliver a desired nucleic acid, for example, bacterial plasmid, viral nucleic acid, HAC, BAC, and the like.

The nucleic acid molecules of the instant invention, individually, or in combination or in conjunction with other drugs, can be used to treat diseases or conditions discussed above. For example, the subject can be treated, or other appropriate cells can be treated, as is evident to those skilled in the art, individually or in combination with one or more drugs under conditions suitable for the treatment.

By “double stranded RNA” or “dsRNA” is meant a double stranded RNA that matches a predetermined gene sequence that is capable of activating cellular enzymes that degrade the corresponding messenger RNA transcripts of the gene. These dsRNAs are referred to as short intervening RNA (siRNA) and can be used to inhibit gene expression (see for example Elbashir et al., 2001, Nature, 411, 494-498; and Bass, 2001, Nature, 411, 428-429). The term “double stranded RNA” or “dsRNA” as used herein refers to a double stranded RNA molecule capable of RNA interference “RNAi”, including short interfering RNA “siRNA” see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.

As used in herein “cell” is used in its usual biological sense, and does not refer to an entire multicellular organism. The cell can, for example, be in vivo, in vitro or ex vivo, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

“MG53,” “MG53 binding protein,” and “MG53 receptor” refers generally to a peptide or protein comprising a full length polypeptide, a domain or fragment thereof, a fusion protein, and/or a chimeric protein.

Oligonucleotides (eg; antisense, GeneBlocs) are synthesized using protocols known in the art as described in Caruthers et al., 1992, Methods in Enzymology 211, 3 19, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS17, 34; Usman et al., 1994, Nucleic Acids Symp. Ser. 31, 163).

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein) have expanded the ability to modify nucleic acid molecules by introducing nucleotide modifications to enhance their nuclease stability as described above. The use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules and/or other chemical or biological molecules). The treatment of subjects with nucleic acid molecules can also include combinations of different types of nucleic acid molecules.

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331 417, and Mesmaeker et al., 1994, Novel Backbone Replacements for Oligonucleotides, in Carbohydrate Modifications in Antisense Research, ACS, 24 39. These references are hereby incorporated by reference herein. Various modifications to nucleic acid (e.g., antisense and ribozyme) structure can be made to enhance the utility of these molecules. For example, such modifications can enhance shelf-life, half-life in vitro, bioavailability, stability, and ease of introduction of such oligonucleotides to the target site, including e.g., enhancing penetration of cellular membranes and conferring the ability to recognize and bind to targeted cells.

Administration of Nucleic Acid Molecules. Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400.

The molecules of the instant invention can be used as pharmaceutical agents. Pharmaceutical agents prevent, inhibit the occurrence, or treat (alleviate a symptom to some extent, preferably all of the symptoms) a disease state in a subject.

The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. By “systemic administration” is meant in vivo systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

Administration routes which lead to systemic absorption include, without limitations: intravenous, subcutaneous, intraperitoneal, inhalation, oral, intrapulmonary and intramuscular. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of nucleic acid molecules include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

An effective amount, pharmaceutically effective dose, therapeutically effective amount, or pharmaceutically effective amount is that dose required to prevent, inhibit the occurrence, or treat (alleviate a symptom to some extent, preferably all of the symptoms) of a disease state or pathological condition. The effective amount depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 1000 mg/kg body weight/day of active ingredients is administered dependent upon potency of the negatively charged polymer. In addition, effective amounts of the compositions of the invention encompass those amounts utilized in the examples to facilitate the intended or desired biological effect.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The formulations can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present. Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Nucleic acid molecules of the invention can also be administered in the form of suppositories, e.g., for rectal administration of the drug or via a catheter directly to the bladder itself. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle. The amount of active ingredient that can be combined with the carrier materials to produce a single dosage form varies depending upon the host treated and the particular mode of administration. Dosage unit forms generally contain between from about 1 mg to about 5000 mg of an active ingredient. It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water. The composition can also be administered to a subject in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591 5; Kashani-Sabet et al., 1992, Antisense Res. Dev., 2, 3 15; propulic et al., 1992, J. Virol., 66, 1432 41; Weerasinghe et al., 1991, J. Virol., 65, 5531 4; Ojwang et al., 1992, Proc. Natl. Acad. Sci. USA, 89, 10802 6; Chen et al., 1992, Nucleic Acids Res., 20, 4581 9; Sarver et al., 1990 Science, 247, 1222 1225; Thompson et al, 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector.

In one aspect the invention features an expression vector comprising a nucleic acid sequence encoding at least one of the nucleic acid molecules of the instant invention. The nucleic acid sequence encoding the nucleic acid molecule of the instant invention is operably linked in a manner which allows expression of that nucleic acid molecule.

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743 7; Gao and Huang 1993, Nucleic Acids Res., 21, 2867 72; Lieber et al., 1993, Methods Enzymol., 217, 47 66; Zhou et al., 1990, Mol. Cell. Biol., 10, 4529 37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3 15; Ojwang et al., 1992, Proc. Natl. Acad. Sci. USA, 89, 10802 6; Chen et al, 1992, Nucleic Acids Res., 20, 4581 9; Yu et al., 1993, Proc. Natl. Acad. Sci. USA, 90, 6340 4; L'Huillier et al., 1992, EMBO J., 11, 4411 8; Lisziewicz et al., 1993, Proc. Natl. Acad. Sci. U.S.A, 90, 8000 4; Thompson et al., 1995, Nucleic Acids Res., 23, 2259; Sullenger & Cech, 1993, Science, 262, 1566).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

A further object of the present invention is to provide a kit comprising a suitable container, the therapeutic of the invention in a pharmaceutically acceptable form disposed therein, and instructions for its use.

In another embodiment, an isolated nucleic acid molecule of the invention comprises a nucleic acid molecule that is a complement of the nucleotide sequence encoding an MG53 polypeptide, or MG53 receptor polypeptide. As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect.

As used herein, “fragments” are defined as sequences of at least 6 (contiguous) nucleic acids or at least 4 (contiguous) amino acids, a length sufficient to allow for specific hybridization in the case of nucleic acids or for specific recognition of an epitope in the case of amino acids, and are at most some portion less than a full length sequence.

The term “host cell” includes a cell that might be used to carry a heterologous nucleic acid, or expresses a peptide or protein encoded by a heterologous nucleic acid. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce one or more of the polypeptide components of the complex of the invention.

“Derivatives” are compositions formed from the native compounds either directly, by modification, or by partial substitution.

“Analogs” are nucleic acid sequences or amino acid sequences that have a structure similar to, but not identical to, the native compound.

Derivatives or analogs of the nucleic acids or proteins of the invention include, but are not limited to, molecules comprising regions that are substantially homologous to the nucleic acids or proteins of the invention, in various embodiments, by at least about 30%, 40%, 50%, 60%, 70%, 80%, 90%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions. See e.g. Ausubel, et al., CURRENT PROTOCOLS IN MOLECULAR BIOLOGY, John Wiley & Sons, New York, N.Y., 1993. Nucleic acid derivatives and modifications include those obtained by gene replacement, site-specific mutation, deletion, insertion, recombination, repair, shuffling, endonuclease digestion, PCR, subcloning, and related techniques.

“Homologs” can be naturally occurring, or created by artificial synthesis of one or more nucleic acids having related sequences, or by modification of one or more nucleic acid to produce related nucleic acids. Nucleic acids are homologous when they are derived, naturally or artificially, from a common ancestor sequence (e.g., orthologs or paralogs). If the homology between two nucleic acids is not expressly described, homology can be inferred by a nucleic acid comparison between two or more sequences. If the sequences demonstrate some degree of sequence similarity, for example, greater than about 30%, 40%, 50%, 60%, 70%, 80%, or 90% at the primary amino acid structure level, it is concluded that they share a common ancestor. For purposes of the present invention, genes are homologous if the nucleic acid sequences are sufficiently similar to allow recombination and/or hybridization under low stringency conditions.

As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA.

Furthermore, one of ordinary skill will recognize that “conservative mutations” also include the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing Methionine (M), Cysteine (C). In addition, sequences that differ by conservative variations are generally homologous.

Descriptions of the molecular biological techniques useful to the practice of the invention including mutagenesis, PCR, cloning, and the like include Berger and Kimmel, GUIDE TO MOLECULAR CLONING TECHNIQUES, METHODS IN ENZYMOLOGY, volume 152, Academic Press, Inc., San Diego, Calif. (Berger); Sambrook et al., MOLECULAR CLONING—A LABORATORY MANUAL (2nd Ed.), Vol. 1-3, Cold Spring Harbor Laboratory, Cold Spring Harbor, N.Y., 1989, and CURRENT PROTOCOLS IN MOLECULAR BIOLOGY, F. M. Ausubel et al., eds., Current Protocols, a joint venture between Greene Publishing Associates, Inc. and John Wiley & Sons, Inc.; Berger, Sambrook, and Ausubel, as well as Mullis et al., U.S. Pat. No. 4,683,202 (1987); PCR PROTOCOLS A GUIDE TO METHODS AND APPLICATIONS (Innis et al. eds), Academic Press, Inc., San Diego, Calif. (1990) (Innis); Arnheim & Levinson (Oct. 1, 1990) C&EN 36-47.

In yet another embodiment, a nucleic acid of the invention is expressed in mammalian cells using a mammalian expression vector. For suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

A polynucleotide can be a DNA molecule, a cDNA molecule, genomic DNA molecule, or an RNA molecule. A polynucleotide as DNA or RNA can include a sequence wherein T (thymidine) can also be U (uracil). If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are substantially complementary to each other when a sufficient number of corresponding positions in each molecule are occupied by nucleotides that can hybridize with each other in order to effect the desired process.

Transformation of a host cell with recombinant DNA may be carried out by conventional techniques as are well known to those skilled in the art. By “transformation” is meant a permanent or transient genetic change induced in a cell following incorporation of new DNA (i.e., DNA exogenous to the cell).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

In any of the embodiments, the nucleic acids encoding an MG53 polypeptide or MG53 receptor can be present as: one or more naked DNAs; one or more nucleic acids disposed in an appropriate expression vector and maintained episomally; one or more nucleic acids incorporated into the host cell's genome; a modified version of an endogenous gene encoding the components of the complex; one or more nucleic acids in combination with one or more regulatory nucleic acid sequences; or combinations thereof. The nucleic acid may optionally comprise a linker peptide or fusion protein component, for example, His-Tag, FLAG-Tag, Maltose Binding Protein (MBP)-Tag, fluorescent protein, GST, TAT, an antibody portion, a signal peptide, and the like, at the 5′ end, the 3′ end, or at any location within the ORF.

In a preferred embodiment, the nucleic acid of the invention comprises a polynucleotide encoding soluble portions of MG53 or an MG53 receptor. Any of the embodiments described herein, can be achieved using standard molecular biological and genetic approaches well known to those of ordinary skill in the art.

Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂, RbCl, liposome, or liposome-protein conjugate can be used. Transformation can also be performed after forming a protoplast of the host cell or by electroporation. These examples are not limiting on the present invention; numerous techniques exist for transfecting host cells that are well known by those of skill in the art and which are contemplated as being within the scope of the present invention.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. The eukaryotic cell may be a yeast cell (e.g., Saccharomyces cerevisiae) or may be a mammalian cell, including a human cell. For long-term, high-yield production of recombinant proteins, stable expression is preferred.

Stem Cell Applications

In another aspect, the present invention encompasses therapeutic methods utiltizing host cells, and stem cells modified according to the methods of the invention, which can be used in transplantation and/or adoptive cellular therapeutic approaches. In one embodiment of this aspect, a stem cell, for example, a cardiac stem cell is isolated from a host, wherein the stem cell is capable of differentiating into a cardiac myocyte, and wherein the isolated stem cell is modified such that it demonstrates a modulated, for example, enhanced, MG53 activity, MG53 gene expression, or modulated MG53 signalling cascade. In a preferred embodiment, the stem cell is contacted with an agent, for example, an MG53 polypeptide, MG53 nucleotide or agent that enhances the MG53 signalling cascade in cardiac cells. The modified stem cell can then be cultured in vitro, and subsequently administered to an individual in need thereof, for example, a patient that has sustained myocardial damage due to ischemia/reperfusion or hypoxia.

A variety of methods are know for the isolation, culture and manipulation of stem cells capable of differentiation into cardiac myocytes. See, for examples, Guo J. et al. Int J Exp Pathol. 2009 June; 90(3):355-64; Patel A. N., and Sherman W., Cell Transplant. 2009; 18(3):243-4; Murtuza B., et al. Tissue Eng Part B Rev. 2009 Jun. 24; Popescu L. M. et al. J Cell Mol. Med. 2009 May; 13(5):866-86. Epub 2009 Apr. 20; Chamuleau S. A. et al. Cardiovasc Res. 2009 Jun. 1; 82(3):385-7. Epub 2009 Apr. 8; the disclosures of which are hereby incorporated by reference in their entirety for all purposes.

Antibodies

The term “antibody” as used herein refers to immunoglobulin molecules and immunologically active portions of immunoglobulin (Ig) molecules, i.e., molecules that contain an antigen-binding site that specifically binds (immunoreacts with) an antigen, comprising at least one, and preferably two, heavy (H) chain variable regions (abbreviated herein as VH), and at least one and preferably two light (L) chain variable regions (abbreviated herein as VL). Such antibodies include, but are not limited to, polyclonal, monoclonal, chimeric, single chain, Fab, Fab′ and F(ab′)2 fragments, and an Fab expression library. The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

Antibodies can be prepared from the intact polypeptide or fragments containing peptides of interest as the immunizing agent. A preferred antigenic polypeptide fragment is 15-100 contiguous amino acids of MG53, or MG53 receptor protein. In one embodiment, the peptide is located in a non-transmembrane domain of the polypeptide, e.g., in an extracellular or intracellular domain. An exemplary antibody or antibody fragment binds to an epitope that is accessible from the extracellular milieu and that alters the functionality of the protein. In certain embodiments, the present invention comprises antibodies that recognize and are specific for one or more epitopes of MG53, or MG53 receptor protein, variants, portions and/or combinations thereof. In alternative embodiments antibodies of the invention may target and interfere with the MG53/MG53 receptor interaction to inhibit signaling.

The preparation of monoclonal antibodies is well known in the art; see for example, Harlow et al., Antibodies: A Laboratory Manual, page 726 (Cold Spring Harbor Pub. 1988). Monoclonal antibodies can be obtained by injecting mice or rabbits with a composition comprising an antigen, verifying the presence of antibody production by removing a serum sample, removing the spleen to obtain B lymphocytes, fusing the lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by techniques well known in the art.

In other embodiments, the antibody can be recombinantly produced, e.g., produced by phage display or by combinatorial methods. Phage display and combinatorial methods can be used to isolate recombinant antibodies that bind to membrane repair polypeptide, MG53, membrane repair polypeptide binding protein, MG53 binding protein, membrane repair polypeptide receptor, and/or MG53 receptor proteins or fragments thereof (as described in, e.g., Ladner et al. U.S. Pat. No. 5,223,409; Fuchs et al. (1991) Bio/Technology 9:1370-1372; Hay et al. (1992) Hum Antibod Hybridomas 3:81-85; Huse et al. (1989) Science 246:1275-1281; Clackson et al. (1991) Nature 352:624-628; Gram et al. (1992) PNAS 89:3576-3580. Human monoclonal antibodies can also be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855). A therapeutically useful antibody to the components of the complex of the invention or the complex itself may be derived from a “humanized” monoclonal antibody. Humanized monoclonal antibodies are produced by transferring mouse complementarity determining regions (CDRs) from heavy and light variable chains of the mouse immunoglobulin into a human variable domain, then substituting human residues into the framework regions of the murine counterparts.

The use of antibody components derived from humanized monoclonal antibodies obviates potential problems associated with immunogenicity of murine constant regions. Techniques for producing humanized monoclonal antibodies can be found in Jones et al., Nature 321: 522, 1986 and Singer et al., J. Immunol. 150: 2844, 1993; Wu T. T. and Kabat, E. A. (1970) J. Exp. Med., 132: 211-250; and Johnson G., Wu, T. T. and Kabat, E. A. (1995) In Paul, S. (ed.), Antibody Engineering Protocols. Humana Press, pp. 1-15, which are incorporated herein by reference. The antibodies can also be derived from human antibody fragments isolated from a combinatorial immunoglobulin library; see, for example, Barbas et al., Methods: A Companion to Methods in Enzymology 2, 119, 1991. In addition, chimeric antibodies can be obtained by splicing the genes from a mouse antibody molecule with appropriate antigen specificity together with genes from a human antibody molecule of appropriate biological specificity; see, for example, Takeda et al., Nature 314: 544-546, 1985. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies can be used to produce single chain antibodies. Single chain antibodies are formed by linking the heavy and light chain fragments of the FIT region via an amino acid bridge, resulting in a single chain polypeptide. Antibody fragments that recognize specific epitopes, e.g., extracellular epitopes, can be generated by techniques well known in the art. Such fragments include Fab fragments produced by proteolytic digestion, and Fab fragments generated by reducing disulfide bridges. When used for immunotherapy, the monoclonal antibodies, fragments thereof, or both may be unlabelled or labeled with a therapeutic agent. These agents can be coupled directly or indirectly to the monoclonal antibody by techniques well known in the art, and include such agents as drugs, radioisotopes, lectins and toxins.

The dosage ranges for the administration of monoclonal antibodies are large enough to produce the desired effect, and will vary with age, condition, weight, sex, age and the extent of the condition to be treated, and can readily be determined by one skilled in the art. Dosages can be about 0.1 mg/kg to about 2000 mg/kg. The monoclonal antibodies can be administered intravenously, intraperitoneally, intramuscularly, and/or subcutaneously.

In certain embodiments of the invention, at least one epitope encompassed by the antigenic peptide is a region of MG53, or MG53 receptor that is located on the surface of the protein, e.g., a hydrophilic region. A hydrophobicity analysis of the protein sequence will indicate which regions of a polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein. A protein of the invention, or a derivative, fragment, analog, homolog or ortholog thereof, may be utilized as an immunogen in the generation of antibodies that immunospecifically bind these protein components.

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol. 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology, 10:779-783 (1992)); Lonberg et al. (Nature, 368:856-859 (1994)); Morrison (Nature, 368:812-13 (1994)); Fishwild et al, (Nature Biotechnology, 14:845-51 (1996)); Neuberger (Nature Biotechnology, 14:826 (1996)); and Lonberg and Huszar (Intern. Rev. Immunol., 13:65-93 (1995)).

Human antibodies may additionally be produced using transgenic nonhuman animals which are modified so as to produce fully human antibodies rather than the animal's endogenous antibodies in response to challenge by an antigen. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 500 mg/kg body weight. Common dosing frequencies may range, for example, from twice daily to once a week.

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., Fab or F(ab)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques intracavity, or transdermally, alone or with effector cells.

Preparations for administration of the therapeutic of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

The compounds, nucleic acid molecules, polypeptides, and antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, intraperitoneal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™. (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

Additional objects and advantages of the present invention will be appreciated by one of ordinary skill in the art in light of the current description and examples of the preferred embodiments, and are expressly included within the scope of the present invention.

### ILLUSTRATIVE EXAMPLES

Discovery of MG53, a muscle specific TRIM family protein. MG53 was isolated using a previously established an immuno-proteomic approach that allows identification of novel proteins involved in myogenesis, Ca²⁺ signaling and maintenance of membrane integrity in striated muscle cells. Briefly, this approach uses a monoclonal antibody library containing ˜6500 clones that was generated from mice immunized with triad-enriched membranes from rabbit skeletal muscle. Antibodies of interest were selected based on the z-line staining patterns of striated muscle sections observed under an immunofluorescence microscope. The target-proteins were purified through antibody-affinity column, and partial amino acid sequences of the purified proteins were obtained. Based on the partial amino acid sequence, the complete cDNA coding for the target gene was isolated from a skeletal muscle cDNA library. Homologous gene screening was then used to search for the presence of different isoforms of the identified genes in other excitable tissues. Finally, transgenic or knockout mouse models were generated to study the in vivo physiological function of genes of interest.

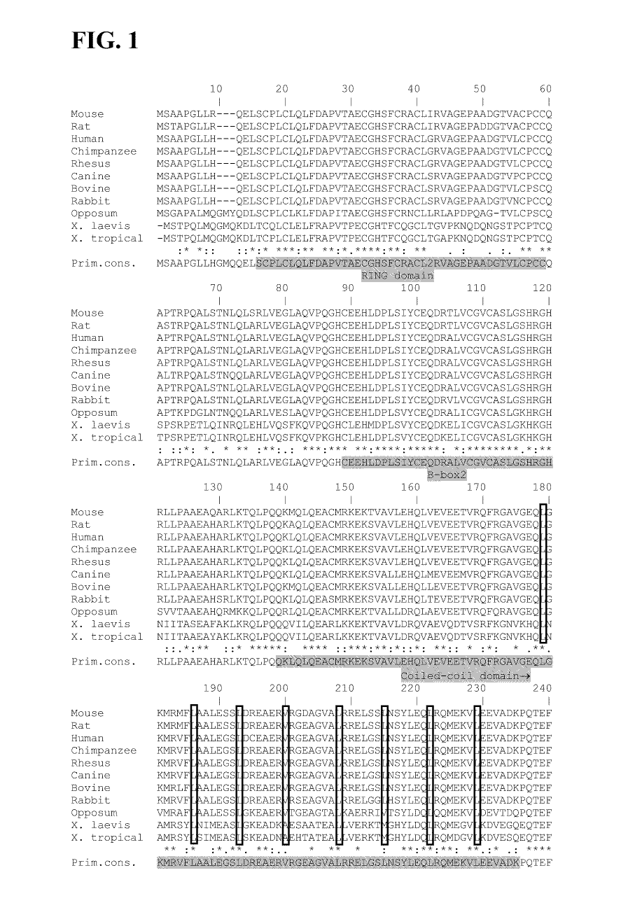

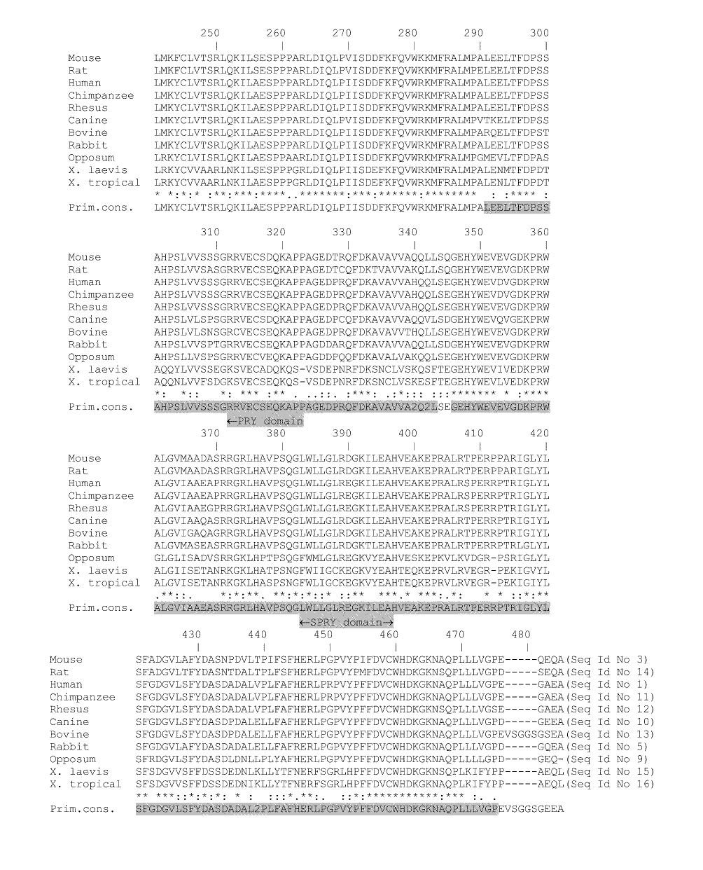

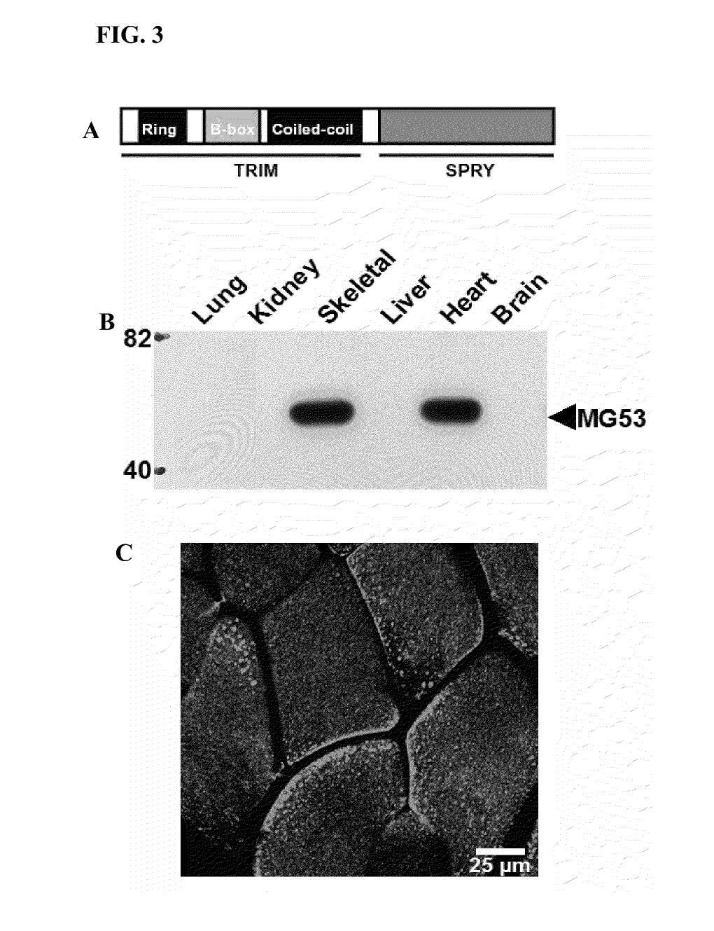

Screening of this immuno-proteomic library for muscle specific proteins led to the identification of an antigen recognized by mAb5259 with a molecular size of 53 kilodaltons (kDa) specifically with striated muscle tissues (FIG. 3B). The protein, “MG53”, was partially purified from rabbit skeletal muscle by a mAb5259 immunoaffinity column and subjected to amino acid sequencing. Skeletal muscle cDNA library screening and genomic database searches identified the predicted amino acid sequences for MG53 and the corresponding mg53 gene on the human 16p11.2 locus. Northern blotting for the mg53 mRNA confirmed specific expression with skeletal and cardiac muscle (FIG. 3C). Domain homology analysis revealed that MG53 contains the prototypical tri-partite motifs that include a Ring, B-box and Coiled-Coil (RBCC) moieties, as well as a SPRY domain at the carboxyl-terminus (FIGS. 1, 2, and 3A). The SPRY domain is a conserved sequence first observed in the ryanodine receptor Ca²⁺ release channel in the sarcoplasmic reticulum of excitable cells. Of the approximately 60 TRIM family members so far identified in various mammalian genomes, 15 members carry a similar SPRY domain following the RBCC domain, and MG53 shows a conserved primary structure with these TRIM sub-family proteins.

MG53 mediates vesicle trafficking in muscle cells. Although there is no membrane-spanning segment or lipid-modification motif in its primary structure, MG53 appears to be primarily restricted to membrane structures in skeletal muscle. Immunohistochemical analysis revealed specific labeling for MG53 in the sarcolemma membrane and intracellular vesicles (FIG. 3D). MG53 is a muscle-specific protein that contains TRIM and SPRY motifs. In previous studies we have established a monoclonal antibody (mAb) library that targets proteins associated with the triad junction in skeletal muscle. Screening of this immuno-proteomic library for muscle specific proteins led to the identification of an antigen named MG53 with a molecular size of 53 kilodaltons (kDa), which was recognized by mAb5259. MG53 was partially purified from rabbit skeletal muscle by an immunoaffinity column conjugated with mAb5259, and subjected to amino acid sequencing. Based on the obtained partial amino acid sequences, cDNAs encoding MG53 were isolated from rabbit and mouse skeletal muscle libraries. Genomic library search identified the corresponding MG53 gene on the human 16p11.2 locus. The predicted amino acid sequences for MG53 in several species are shown in FIG. 1.

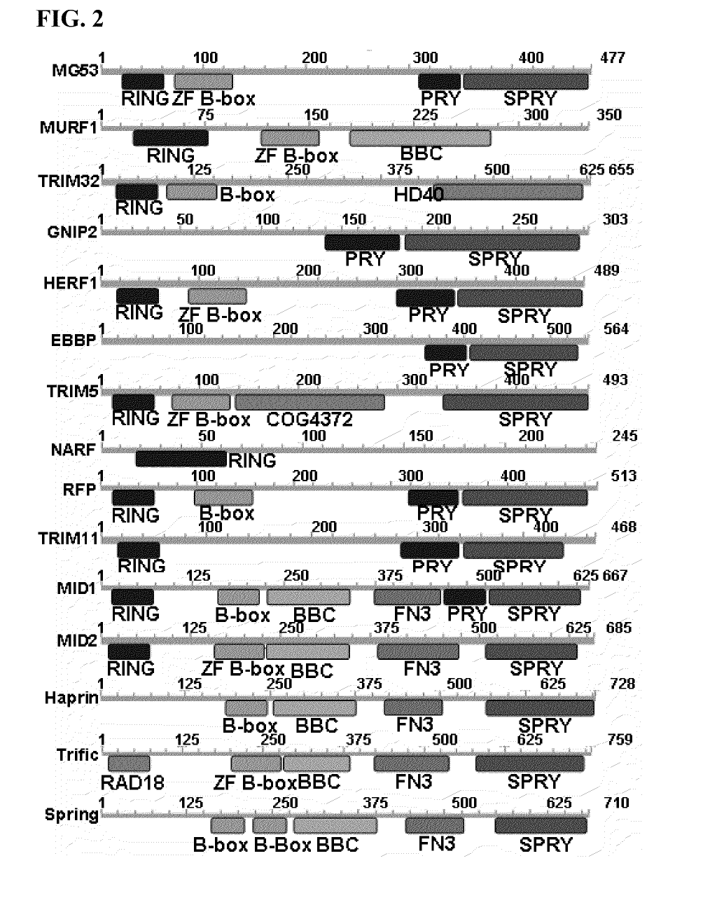

Domain homology analysis revealed that MG53 contains the prototypical TRIM signature sequence of RBCC plus a SPRY domain at the carboxyl-terminus, and thus belongs to the TRIM/RBCC family (FIG. 1). Of the approximately 60 TRIM family members so far identified in the mammalian genomes, 15 members carry a similar SPRY domain following the RBCC domain, and MG53 shows a conserved primary structure with these TRIM sub-family proteins (FIG. 2). However, surprisingly and unexpectedly our studies indicate that MG53 is the only TRIM family protein of those in FIG. 2 that demonstrate membrane repair function.

Western blot assay confirms the muscle-specific expression of MG53 in mouse tissues (FIG. 3B). Although there is no membrane-spanning segment or lipid-modification motif in its primary structure, MG53 appears to be primarily restricted to membrane structures in skeletal muscle Immunohistochemical analysis with mAb5259 showed specific labeling for MG53 in the sarcolemmal and TT membranes in transverse sections of skeletal muscle fibers (FIG. 3C). Moreover, transverse sections revealed localized concentration of MG53 near the sarcolemmal membrane, with a broader staining pattern than is typically observed for integral membrane proteins of the sarcolemma Thus, MG53 is a muscle specific TRIM family protein that displays a unique subcellular distribution pattern for a TRIM family protein.

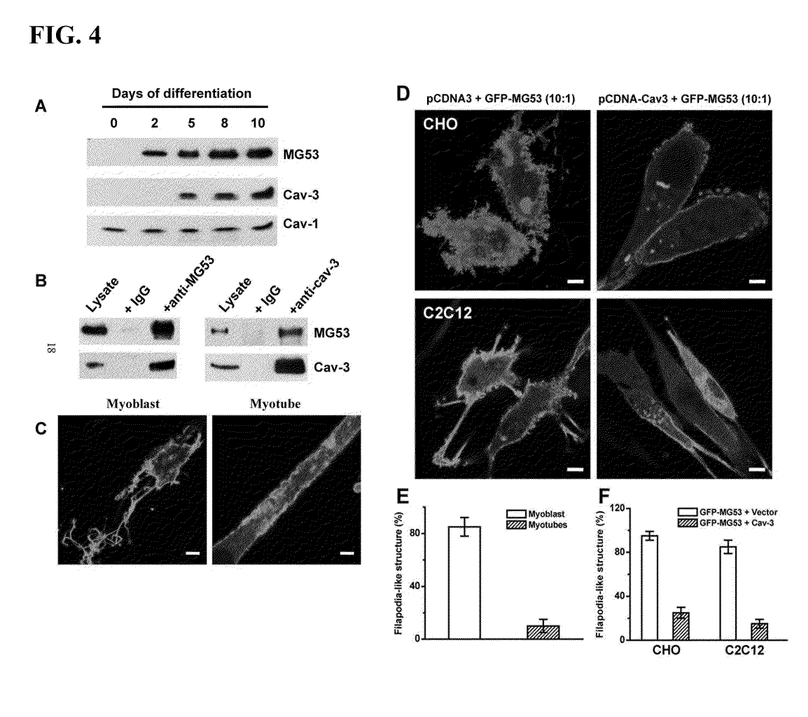

Expression of MG53 is essential to maintain normal cardiac membrane integrity. Defects in mg53−/− mice are not limited to skeletal muscle fibers. During injection of Evans blue dye ˜50% of the mg53−/− mice would die within 16 hours of injection compared to none of the wild type animals injected. Postmortem examination of mg53−/− hearts revealed extensive labeling of cardiac muscle fibers with Evans blue, even in absence of exercise stress (FIG. 4). We also found that exercise would greatly exacerbate the extent of Evans blue staining in mg53−/− hearts.

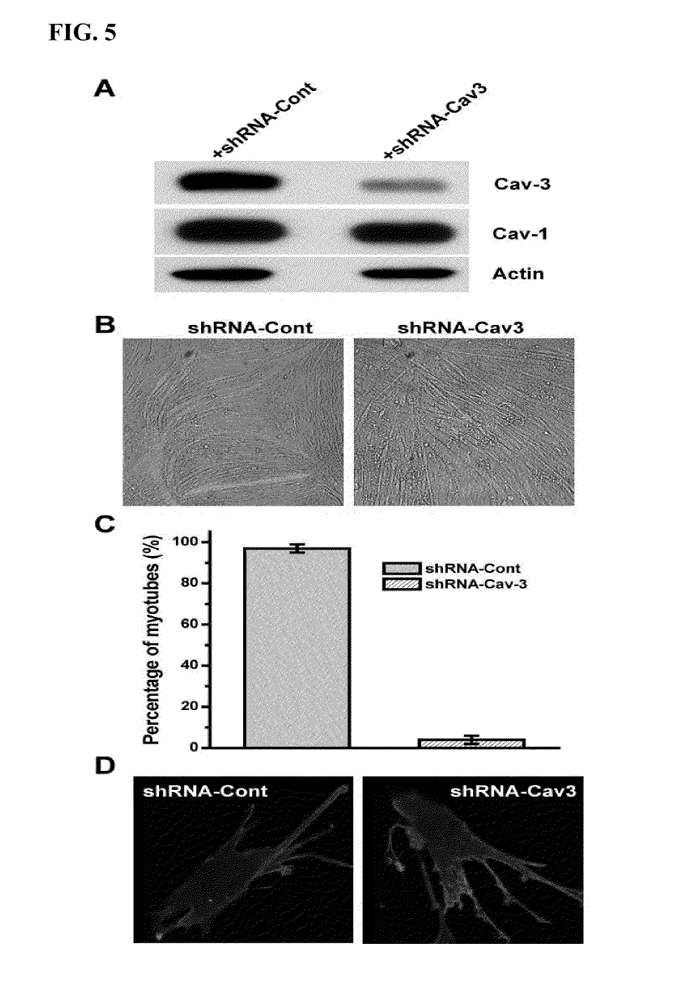

Loss of MG53 increases susceptibility to cardiac ischemia reperfusion injury (FIG. 5). Hearts from wild type (WT) and mg53−/− (mg53KO) mice were isolated and perfused on a Langendorff apparatus. Global ischemia was induced for about 30 minutes by cessation of perfusate flow. The damage produced in the heart following restoration of perfusate flow (time 0) was measured by enzymatic assays for (a) creatine kinase (CK) or (b) lactate dehydrogenase (LDH). Hearts from mg53−/− mice (dashed lines) show more damage than WT (solid lines). Data is presented as mean±S.D. for each listed time point.

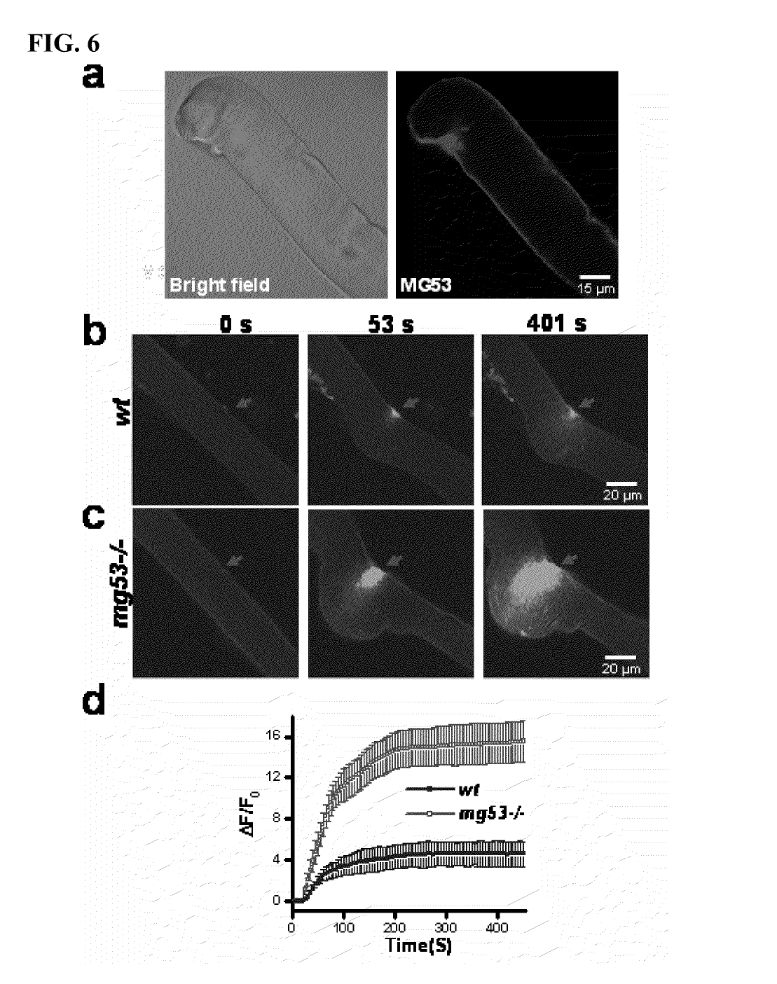

Because caveolin-3 is developmentally regulated (FIG. 6A) and can interact with MG53 (FIG. 6B), we tested whether MG53-induced filapodia-like structure in C2C12 myoblasts could be influenced by the overexpression of caveolin-3. As shown in FIG. 6D, concurrent overexpression of caveolin-3 and MG53 in either C2C12 myoblasts lead to remarkable inhibition of the appearance of filapodia-like structures associated with GFP-MG53 overexpression. On average, C2C12 myoblasts transfected with caveolin-3 and GFP-MG53 (in a ratio of about 10:1) exhibited an 82±6% reduction in the appearance of filapodia-like structures, respectively (FIGS. 6E and F). These results suggest that caveolin-3 represents one of the molecular regulators of MG53-mediated membrane fusion events.

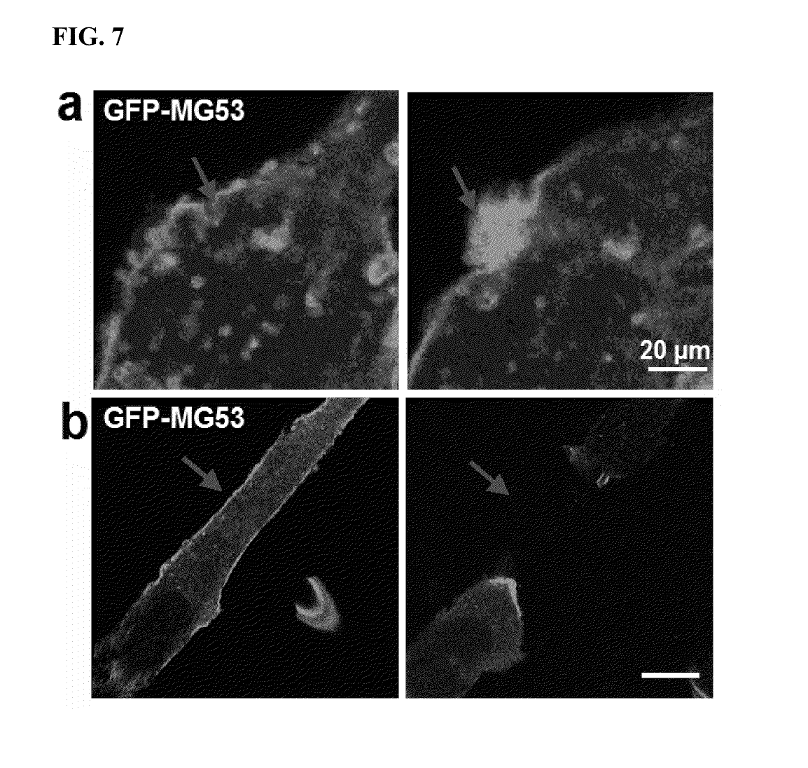

To further investigate the role of caveolin-3 in the subcellular distribution of MG53 and the formation of filapodia-like structures, a caveolin-3 shRNA plasmid (Table 1) was constructed that includes an independent red fluorescence protein expression cassette to provide a marker for shRNA transfected cells. Western blot analysis shown in FIG. 7A reveals that the shRNA-cav3 probe is highly efficient at suppressing the caveolin-3 expression in CHO cells transiently transfected with the caveolin-3 cDNA without affecting the expression of caveolin-1.

TABLE 1
Oligos for constructing the shRNA
for MG53 and Caveolin-3.

| Plasmid | | Inserted oligos |
|---|---|---|
| Scrambled | sense | 5′-GTA CCT CGC CTG CCG TCC AAA |
| shRNA | (SEQ ID | GTT GTA ATC AAG AGT TAC AAC TTT |
| for MG53 | NO. 18) | GGA CGG CAG GCT TTT TGG AAA-3′ |
| | antisense | 5′-AGC TTT TCC AAA AAG CCT GCC |
| | (SEQ ID | GTC CAA AGT TGT AAC TCT TGA TTA |
| | NO. 19) | CAA CTT TGG ACG GCA GGC GAG-3′ |
| shRNA | sense | 5′-GTA CCT CGA GCT GTC AAG CCT |
| for | (SEQ ID | GAA CTC TTC AAG AGA GAG TT CAG |
| MG53 | NO. 20) | GCT TGA CAG CTC TTT TTG GAA A-3′ |
| | antisense | 5′-AGC TTT TCC AAA AAG AGC TGT |
| | (SEQ ID | CAA GCC TGA ACT CTC TCT TGA AGA |
| | NO. 21) | GTT CAG GCT TGA CAG CTC GAG-3′ |
| Scrambled | sense | 5′-GAT CCG CGG AGA CAT AGC CTG |
| shRNA | (SEQ ID | TAA TTC AAG AGA TTA CAG GCT ATG |
| for Cav-3 | NO. 22) | TCT CCG CTT TTT TAC CGG TG-3′ |
| | antisense | 5′-AAT TCA CCG GTA AAA AAG CGG |
| | (SEQ ID | AGA CAT AGC CTG TAA TCT CTT GAA |
| | NO. 23) | TTA CAG GCT ATG TCT CCG CG-3′ |
| shRNA | sense | 5′-GAT CCG GAC ATT CAC TGC AAG |
| for | (SEQ ID | GAG TTC AAG AGA CTC CTT GCA GTG |
| Cav-3 | NO. 24) | AAT GTC CTT TTT TAC CGG TG-3′ |
| | antisense | 5′-AAT TCA CCG GTA AAA AAG GAC |
| | (SEQ ID | ATT CAC TGC AAG GAG TCT CTT GAA |
| | NO. 25) | CTC CTT GCA GTG AAT GTC CG-3′ |

While C2C12 myoblasts transfected with a non-specific shRNA exhibit a normal differentiation pattern as shown by the abundant red-fluorescent labeled myotubes in the left panel of FIG. 7B, acute suppression of caveolin-3 could significantly inhibit the differentiation of C2C12 myoblasts into myotubes (FIG. 7B, right panel). On average, less than about 10% of the shRNA-cav3 transfected myoblasts marked by red-fluorescence could differentiate into mature myotubes at day 6 after application of differentiation media (FIG. 7C). This result is consistent with previous studies by other investigators, which showed that the expression of caveolin-3 is essential for differentiation of C2C12 myotubes.

Confocal microscopic imaging showed that transfection of shRNA-cav3 into C2C12 myoblasts did not appear to affect the subcellular distribution of GFP-MG53 expressed in these cells (FIG. 7D). In particular, the distinct pattern of vesicular distribution of GFP-MG53 and filapodia-like membrane structures remained unaffected by the transient transfection with either shRNA-cav3 or the non-specific shRNA. This result is consistent with the lack of expression of caveolin-3 in the myoblast stage of C2C12 cells.

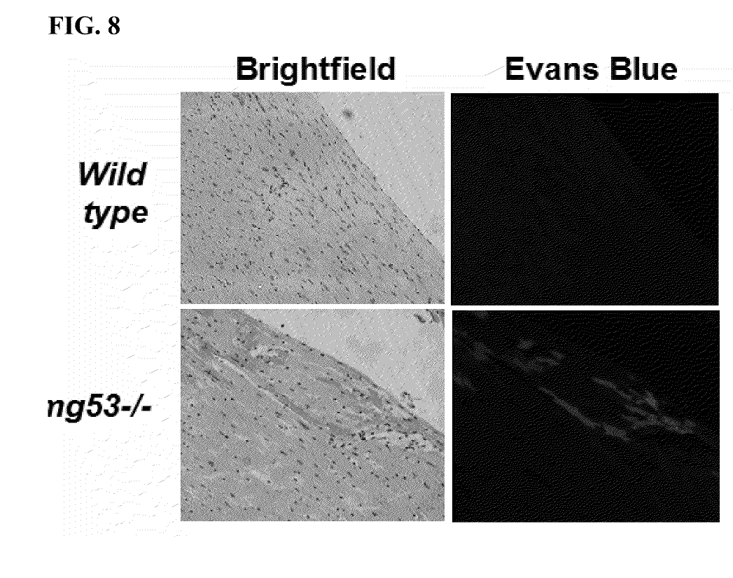

Expression of MG53 is essential to maintain normal cardiac membrane integrity. Defects in mg53−/− mice are not limited to skeletal muscle fibers. During injection of Evans blue dye ˜50% of the mg53−/− mice would die within 16 hours of injection compared to none of the wild type animals injected. Postmortem examination of mg53−/− hearts revealed extensive labeling of cardiac muscle fibers with Evans blue, even in absence of exercise stress (FIG. 8). We also found that exercise would greatly exacerbate the extent of Evans blue staining in mg53−/− hearts.

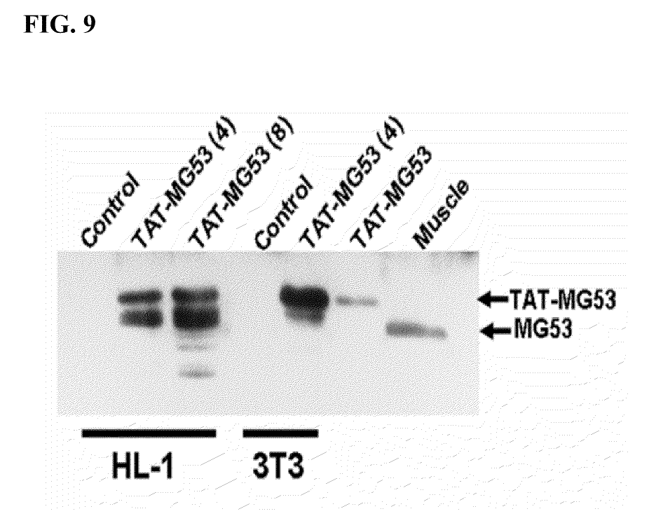

Recombinant human TAT-MG53 can penetrate cells of different origins. In order for MG53 to function it must be present intracellularly. In order to demonstrate that recombinant MG53 can be translocated across the cellular membrane in therapeutically significant amounts HL-1 cardiomyocytes and 3T3 fibroblasts were incubated with about 4 or 8 μg/mL recombinant human TAT-MG53 for approximately 15 minutes at about 37° C. (FIG. 9). The cells were washed three times in a buffered salt solution and then lysed for western blot analysis. Western blot shows that control cells (control) do not contain endogenous MG53, however those incubated with TAT-MG53 contain ample intracellular TAT-MG53. Note that TAT-MG53 is slightly larger than MG53 visualized from skeletal muscle extract (muscle) due to the addition of the TAT cell penetrating peptide to the protein. Multiple bands may be generated by intracellular processing for the TAT-MG53 fusion protein. Therefore, in a preferred embodiment of the MG53 polypeptide therapeutic, the present invention comprises a recombinant polypeptide comprising a TAT polypeptide portion and an MG53 polypeptide portion, wherein the TAT and MG53 polypeptide portions are present in a single, contiguous polypeptide chain.

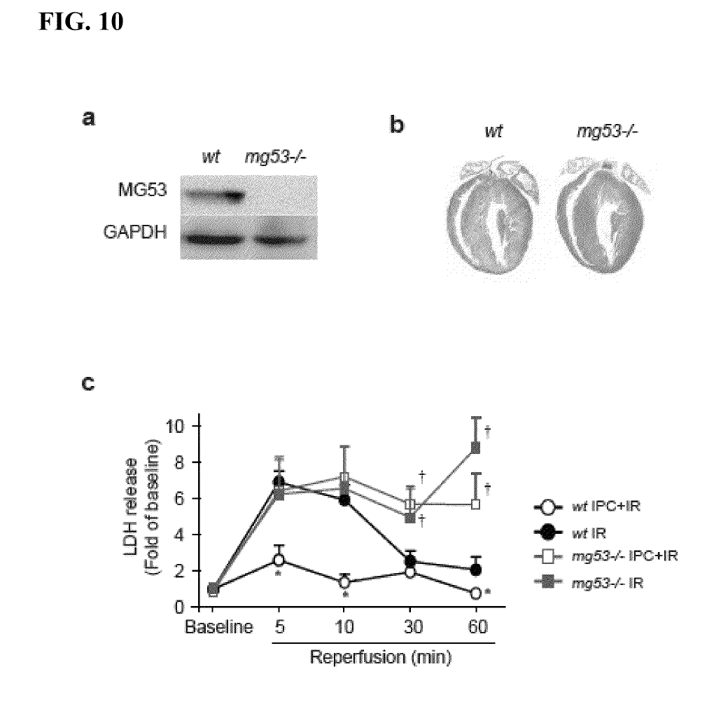

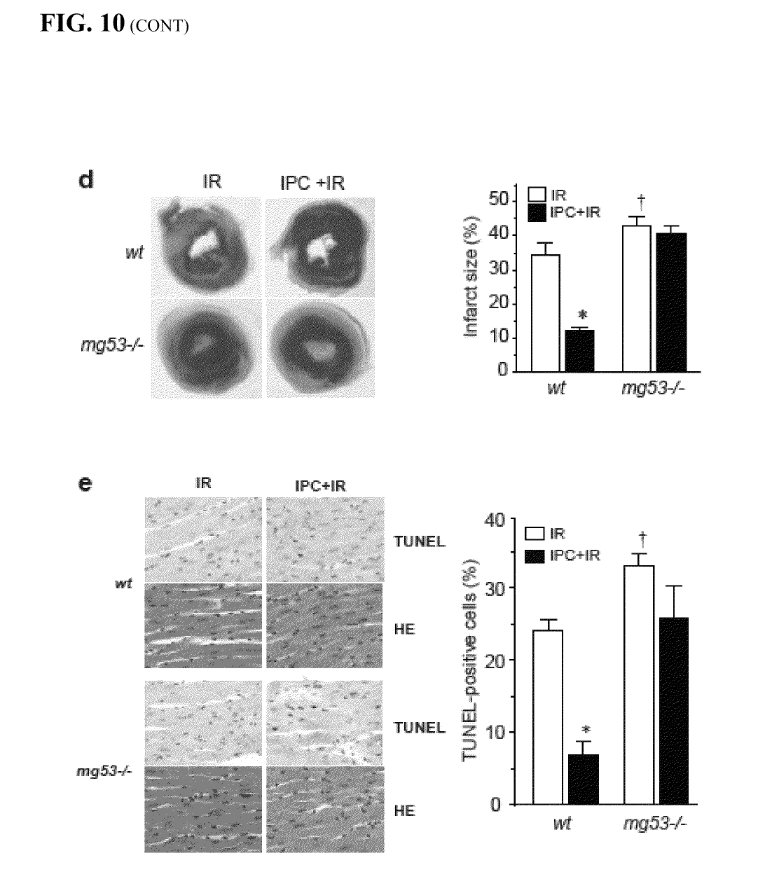

To further elucidate the role of MG53 in IPC-mediated cardiac protection, a gene-targeted MG53 knockout mouse model was utilized⁷. Western blotting confirmed the lack of MG53 protein in myocardium from MG53-deficient mice (FIG. 10a). Under physiological resting conditions, there were no morphological or functional differences between wild type (wt) and mg53−/− mice at the age of 2-3 months (FIG. 10b) (see Table 1). However, IR-induced myocardial damage during Langendorff perfusion was markedly exaggerated in the mg53−/− heart (FIG. 10c). The appearance of lactate dehydrogenase (LDH) in the perfusate following IR injury provides a direct index of damage to the sarcolemmal membranes in the isolated heart. Consistent with previous findings, the wt heart showed a transient LDH increase following IR that was markedly reduced by IPC; in sharp contrast, the mg53−/− heart showed a sustained elevation of LDH release regardless of IPC, suggesting a compromised capacity for membrane repair in the mg53−/− myocardium¹⁷. Similarly, IPC did not protect the mg53−/− heart by ameliorating IR-induced infarction (FIG. 10d). While IPC profoundly reduced the degree of apoptosis in wt heart (FIG. 10e), TUNEL assay revealed no effect of IPC on apoptosis in the mg53−/− heart. Since the TUNEL assay measures apoptotic events that do not involve breakdown of the sarcolemmal membrane, which occurs during necrotic cell death, this finding suggests that MG53-mediated cardioprotection is attributable at least in part to intracellular events that promote cell survival.

TABLE 1
Cardiac function and morphology parameters (n = 8)

| | | LVIDd | LVPWd | LVIDs | LVPWs | | | | | HW/BW |
|---|---|---|---|---|---|---|---|---|---|---|
| | HR (bpm) | (mm) | (mm) | (mm) | (mm) | EF (%) | FS (%) | HW (g) | BW (g) | (g/kg) |
| wt | 635.6 ± 19.1 | 3.12 ± 0.07 | 0.71 ± 0.02 | 1.76 ± 0.03 | 1.11 ± 0.04 | 78.86 ± 1.38 | 41.64 ± 1.27 | 0.16 ± 0.01 | 23.40 ± 1.44 | 6.88 ± 0.23 |
| mg53-/- | 624.2 ± 13.3 | 2.89 ± 0.14 | 0.67 ± 0.03 | 1.61 ± 0.08 | 1.11 ± 0.04 | 81.59 ± 0.55 | 44.12 ± 0.57 | 0.15 ± 0.01 | 23.10 ± 0.37 | 6.61 ± 0.40 |

HR, heart rate.
LVID, left ventricle internal diameter.
LVPW, left ventricle posterior wall thickness.
EF, ejection fraction.
FS, fraction shortening.
HW, heart weight.
BW, body weight.
d, diastolic.
s, systolic
Values are mean ± s.e.m..

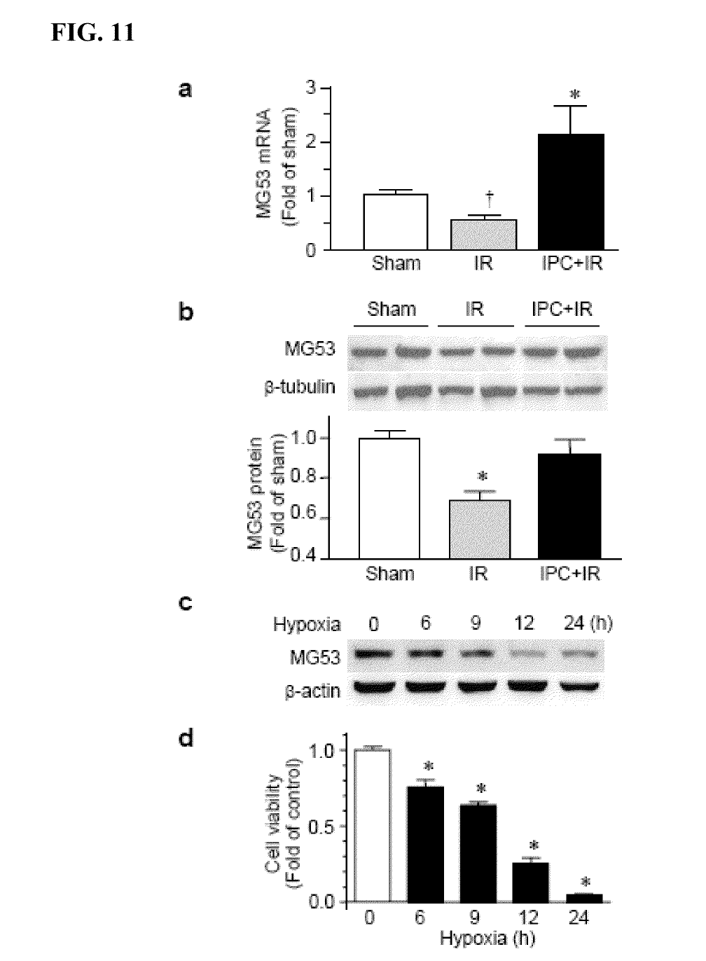

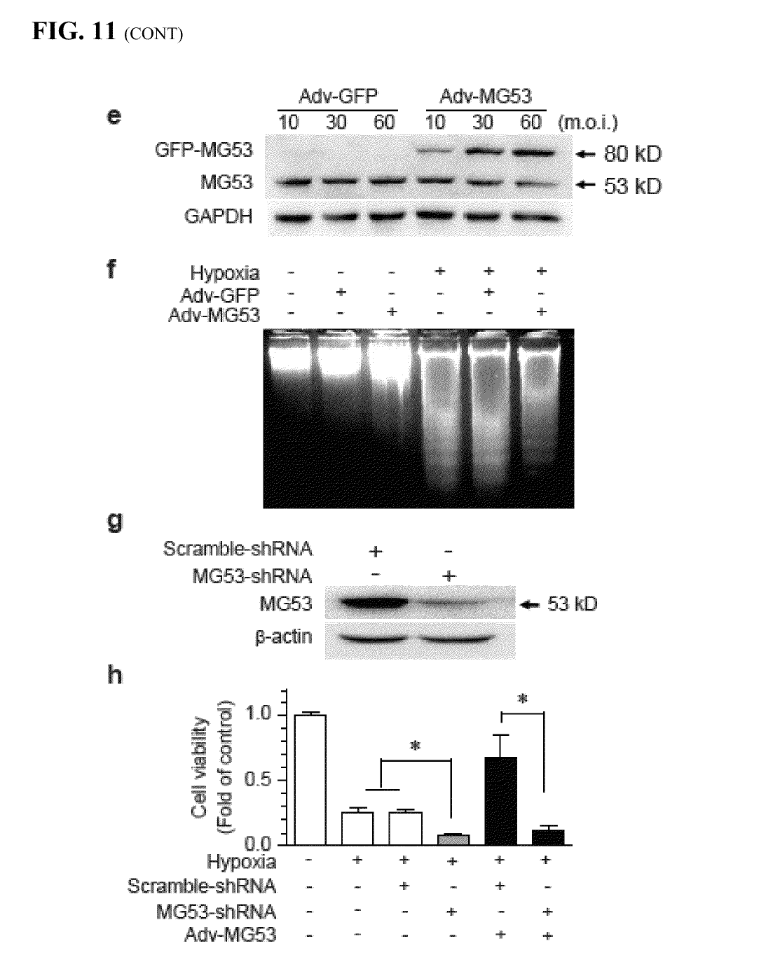

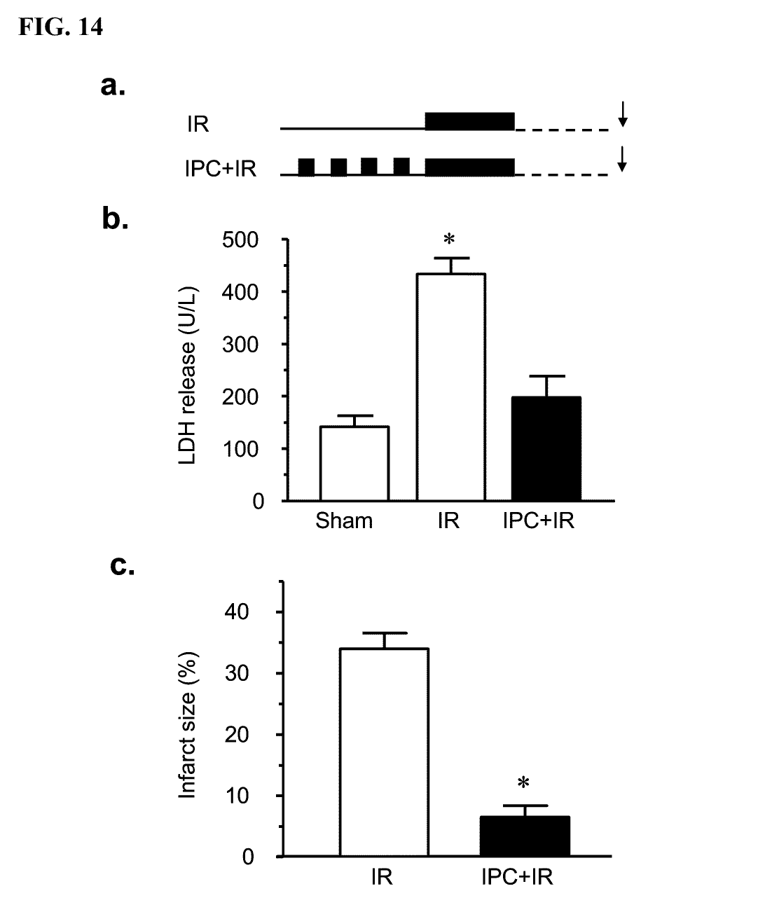

Real-time quantitative PCR showed that IR decreased MG53 mRNA levels in an in vivo rat IR model (FIG. 11a) (see FIG. 14). IPC resulted in an elevation of MG53 mRNA transcripts. Western blotting of parallel samples showed a marked reduction of MG53 protein induced by IR, with a restoration to normal MG53 protein levels if IPC was conducted before the IR injury (FIG. 11b). In vitro experiments using isolated rat neonatal cardiomyocytes revealed that exposure to hypoxic conditions led to a progressive decline in MG53 expression (FIG. 11c) that correlated with decreased cell viability over the same time course (FIG. 11d). Since the mechanisms contributing to ischemia/hypoxia-induced changes in MG53 expression merit further study, MG53 was assayed to determine whether it directly affects cardiomyocyte survival following ischemic/hypoxic stress.

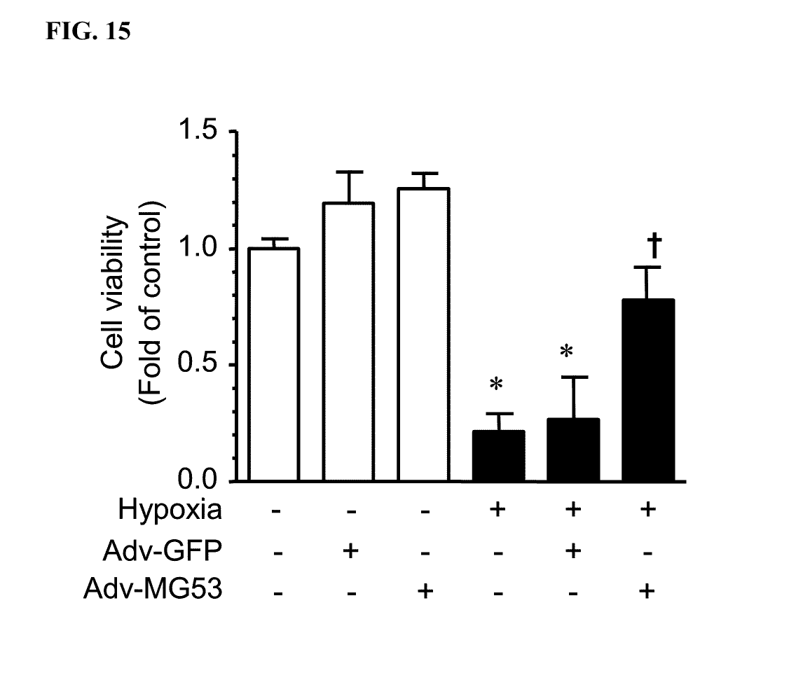

Adenovirus-mediated overexpression of GFP-MG53 fusion protein in cultured neonatal rat cardiomyocytes (FIG. 11e) had a clear protective effect against hypoxia-induced apoptosis. Overexpression of GFP-MG53 profoundly reduced hypoxia-induced cell death (see FIG. 15) including apoptotic cell death assayed by DNA laddering (FIG. 11f). In addition, infection with an adenovirus containing a small-hairpin (sh) RNA that effectively reduced MG53 expression (FIG. 11g) exacerbated hypoxia-induced reduction of cell viability and also eliminated the protective effect of GFP-MG53 overexpression (FIG. 11h). These results indicate that MG53 plays an important role in protection of cardiomyocytes from hypoxia-induced damage. The direct correlation of MG53 expression level with cardiomyocyte viability indicates that the increased vulnerability of the mg53−/− heart to IR is likely to be a direct consequence of the lack of MG53 rather than an adaptive response in the mg53−/− mouse.

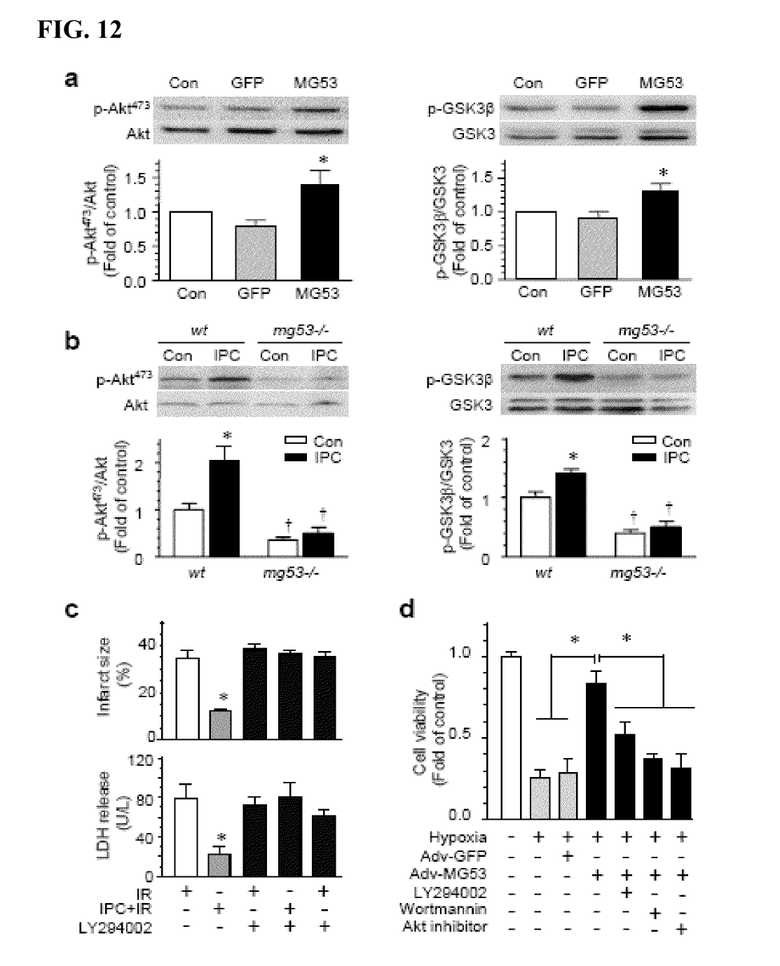

To elucidate the mechanism underlying MG53-mediated cardioprotection, biochemical assays were conducted to determine whether MG53 affects specific survival kinase pathways in cardiomyocytes. Overexpression of MG53 significantly elevated the phosphorylation levels of several key pro-survival kinases, including Akt and GSK3β (FIG. 12a). These kinases were also abundantly activated by IPC in wt mouse heart (FIG. 12b). In the mg53−/− heart, the basal phosphorylation levels of Akt and GSK3β• were notably lower than in their wt counterparts. Strikingly, IPC failed to increase the phosphorylation levels of either Akt or GSK3β in the MG53-deficient heart. The reduced basal phosphorylation of these kinases may explain the reduced tolerance of the MG53-deficient heart to IR injury. The lack of IPC protection in the mg53−/− heart may be linked to the defective pro-survival PI3K-Akt-GSK3β signaling cascade. Indeed, inhibition of the PI3K-Akt axis by a PI3K inhibitor, LY294002, completely abolished IPC-mediated protection in the wt heart. In the wt heart, the IPC-mediated reduction in infarct size (upper panel) and decrease in LDH release (lower panel) were prevented by inhibition of PI3K activity (FIG. 12c). Furthermore, cardiomyocyte protection mediated by MG53-overexpression was largely abrogated by blockade of the PI3K-Akt axis with PI3K inhibitors (LY294002 and wortmannin) or an Akt inhibitor (FIG. 12d). Together, these results show that MG53 is an essential component of the IPC survival pathway.

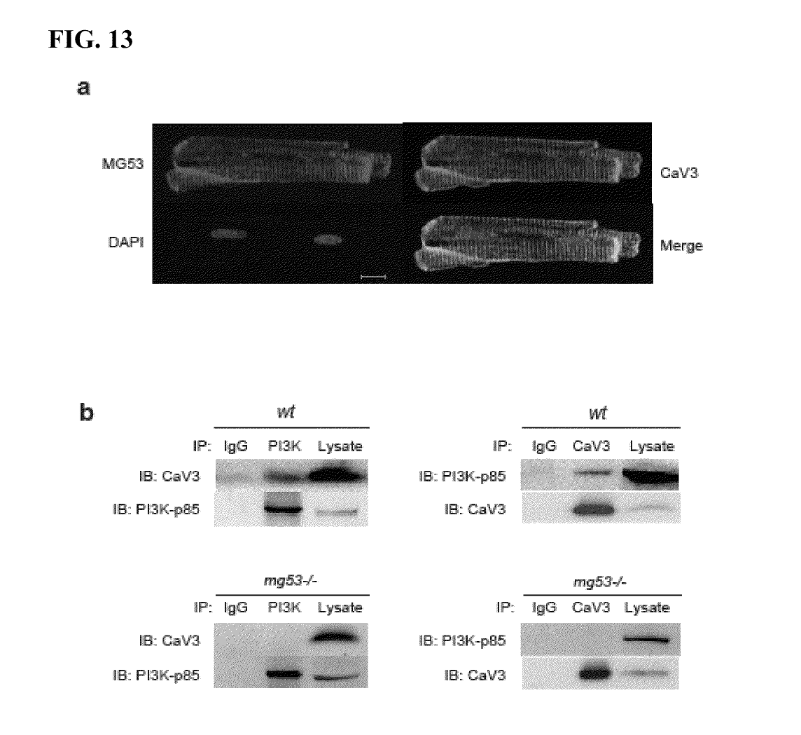

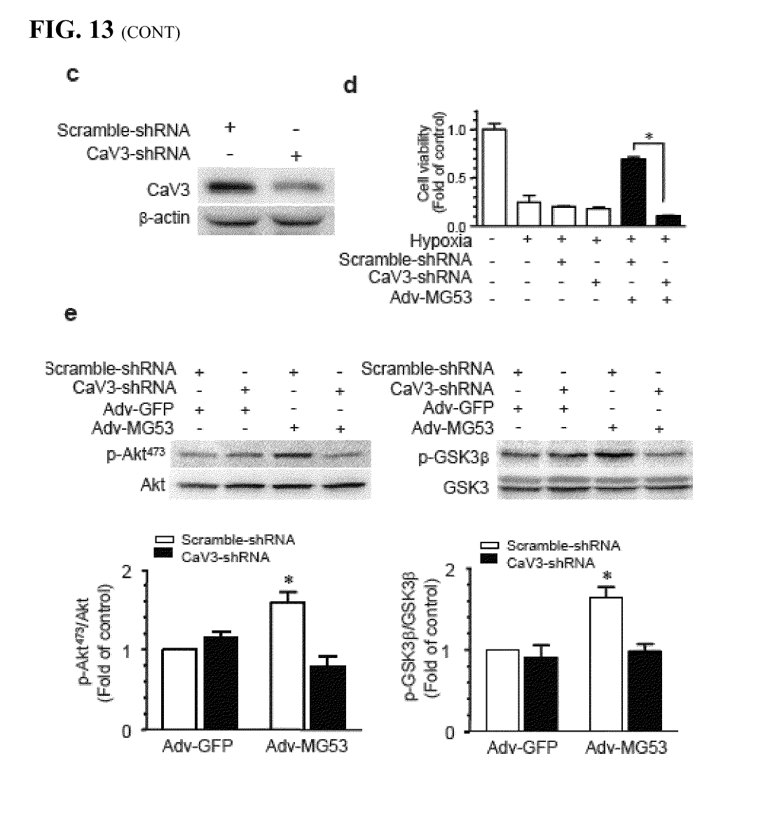

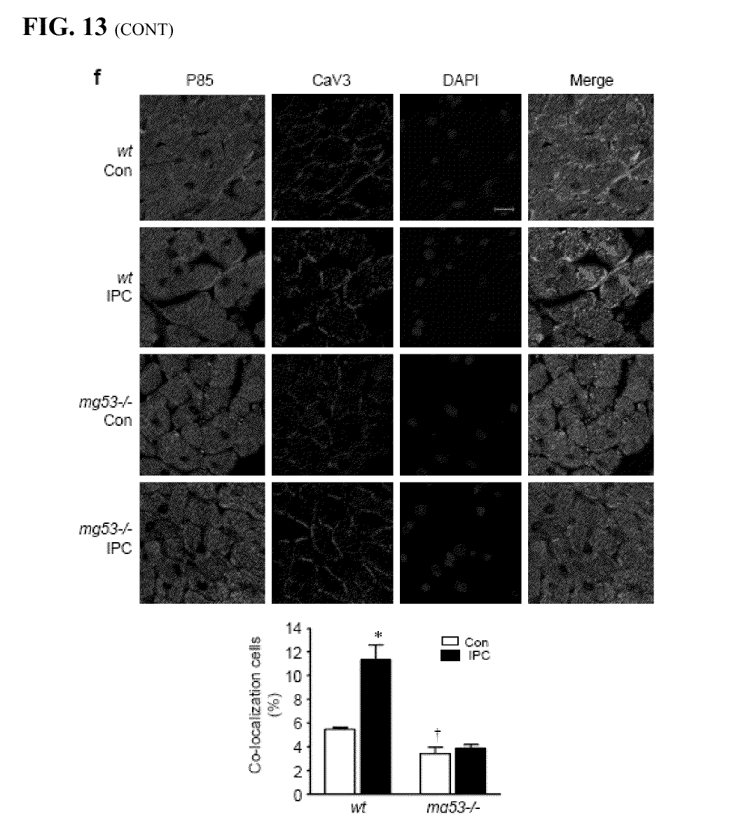

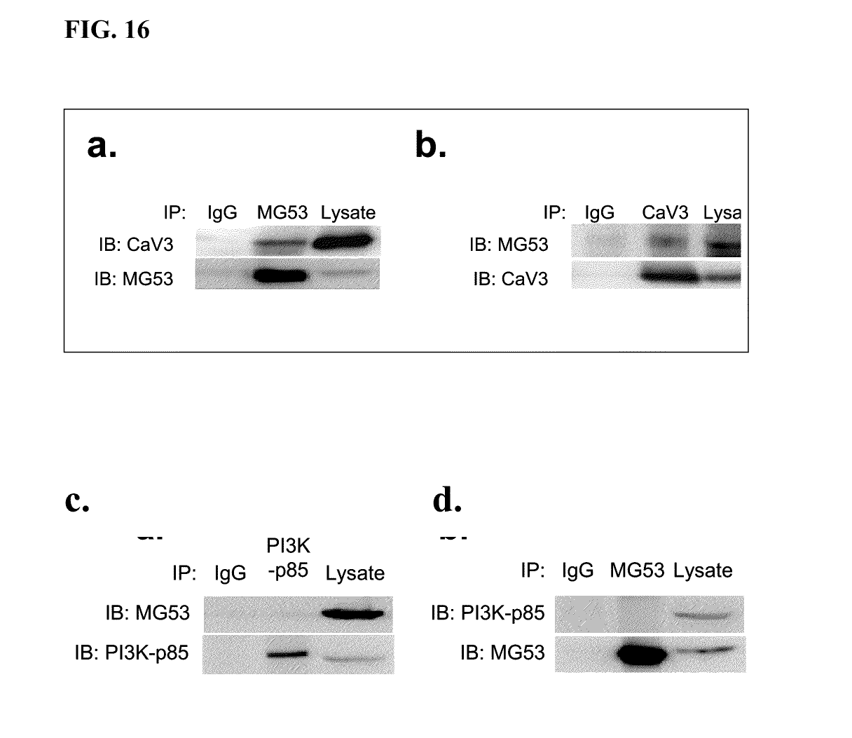

Previous studies have shown that IPC-mediated cardioprotection involves the action of CaV3 at caveolae structures on the cell membrane^{18,19}. Our recent studies have demonstrated that MG53 forms a functional complex with CaV3 in skeletal muscle to regulate the membrane trafficking and remodeling process under both physiological⁸ and pathophysiological conditions⁹. This physical interaction between MG53 and CaV3 also occurs in native cardiac muscle (see FIG. 16) Immunohistochemical staining revealed that MG53 and CaV3 displayed an overlapping distribution pattern in adult cardiomyocytes (FIG. 13a). To test if the MG53-CaV3 complex directly interacts with components of the pro-survival PI3K-Akt-GSK3β signaling pathway, co-immunoprecipitation assays were performed. This revealed a physical interaction between the p85 subunit of PI3K and CaV3 (FIG. 13b, upper panel). Interestingly, this interaction was disrupted in the mg53−/− heart (FIG. 13b, lower panel), suggesting that MG53 is required for CaV3 interaction with PI3K.

The adenoviral delivery of shRNA against CaV3 was used to test if knockdown of CaV3 expression influences MG53-mediated survival of cardiomyocytes following hypoxia. Western blot analysis revealed that CaV3-shRNA effectively suppressed CaV3 expression in cultured cardiomyocytes (FIG. 13c), and eliminated the protective effect of MG53 overexpression against hypoxia-induced cell death (FIG. 13d). This effect on cell survival directly correlated with the degree of phosphorylation of Akt and GSK3β. Clearly, MG53 overexpression-induced hyper-phosphorylation of Akt and GSK3β was reduced after silencing CaV3 expression (FIG. 13e). These data suggest that a functional complex of MG53-CaV3-PI3K participates in activation of the pro-survival PI3K-Akt-GSK3β signaling pathway to produce the cardioprotective response to IPC.

While these biochemical studies indicate a role for MG53 function in survival kinase function, further studies in intact cells provide direct evidence that MG53 is essential for PI3K clustering and activation. Under resting conditions, CaV3 was enriched in the vicinity of cell sarcolemmal membranes, whereas the p85 subunit of PI3K was largely distributed in the cytosol with a minor enrichment around sarcolemmal membranes in the wt heart (FIG. 13f). In the mg53−/− myocardium, PI3K localization was altered and staining appeared to be more intense but uniformly distributed. Importantly, while IPC was able to promote PI3K translocation to the plasma membrane in wt hearts, it did not do so in mg53−/− hearts (FIG. 13f), indicating that IPC-induced translocation of PI3K and subsequent interaction of PI3K with CaV3 requires the presence of MG53. On average, the percentage of cells in wt hearts that showed co-localization of PI3K and CaV3 increased from 5.46±0.18% to 11.31±1.25% following IPC treatment. Taken together, our data indicates that through its function in vesicle trafficking, MG53 forms a functional complex with CaV3, which is obligatory for the recruitment of the survival kinase, PI3K, to the caveolae-membrane domain and subsequent activation of the major survival signaling cascade, PI3K-Akt-GSK3., resulting in myocardial protection.

The present studies show, surprisingly and unexpectedly, that hearts lacking MG53 are more vulnerable to IR injury, and overexpression of MG53 provides cardioprotective benefits. The mg53−/− heart has defective PI3K-Akt-GSK3β signaling pathway and does not respond to IPC-mediated cardioprotection. These present findings suggest that MG53-mediated cardioprotection is attributable to intracellular events that promote cell survival. Indeed, reduced MG53 expression prevents IPC-induced phosphorylation of Akt and GSK3β, while elevated MG53 levels enhance phosphorylation of both kinases. MG53 controls the PI3K-Akt-GSK3• survival pathway through its role in membrane trafficking by interaction with CaV3 to recruit PI3K to caveolae signaling domains. Although our previous studies established MG53 as an essential component of the membrane repair machinery^{7,9}, MG53-mediated cardioprotection is, at least in part, independent of this repair function, since IPC as well as overexpression of MG53 profoundly suppresses apoptotic events that do not involve breakdown of the sarcolemmal membrane.

Due to the limited regenerative capacity of cardiomyocytes, ameliorating ischemia-induced myocardial damage is an important therapeutic target in the treatment of ischemic heart disease. The PI3K-Akt-GSK3β pathway has been implicated in IPC-mediated survival of cardiomyocytes³⁻⁶. Modulating the membrane trafficking and clustering of these survival factors can have beneficial effects for protection from injury to the heart. Since IPC is a powerful and effective protective cellular mechanism against stress-induced myocardial damage, the identification and mechanistic characterization of MG53 as a primary component of the cardiac IPC response establishes it and its receptors as therapeutic avenues for treatment of cardiovascular diseases, particularly ischemic heart disease.

Recombinant MG53 Protein in the Treatment of Ischemia Reperfusion Injury in the Heart

Ischemic heart disease caused by coronary arteriosclerosis remains as the single largest cause of mortality in western countries. As a result of arteriosclerosis or cardiac surgery, blockade of heart blood flow leads to acute myocardial infarction that causes two types of myocardial damage, including ischemic injury induced by the initial loss of blood flow and reperfusion injury by the restoration of oxygenated blood flow. Although the myocardium can tolerate brief exposure to ischemia by switching metabolism to anaerobic glycolysis, persistent ischemia results in irreversible myocardial damage leading to profound myocyte death and permanent loss of contractility. While it has been hypothesized that defective cell membrane repair can contribute to many types of human diseases, including heart failure, there has been limited effort in protein therapeutics targeting prevention of membrane damage in cardiomyocytes.

We recently discovered that MG53, a muscle-specific TRIM-family protein, is an essential component of the acute membrane repair machinery in striated muscle. MG53 acts as a sensor of oxidation to nucleate recruitment of intracellular vesicles to the injury site for membrane patch formation. We found that MG53 can interact with dysferlin to facilitate its membrane repair function, and the membrane trafficking function of MG53 can be modulated through a functional interaction with caveolin3. Our data indicate that a molecular complex formed by MG53, dysferlin and caveolin3 is essential for repair of membrane damage, thus providing a therapeutic target for treatment of muscular and cardiovascular diseases.

Further studies showed that recombinant MG53 protein can protect against membrane damage even when applied to the external space surrounding target cells. These findings could be translated into a significant translational approach to treat myocardial ischemia/reperfusion (I/R) injury. In an effort to develop MG53 as a therapeutic intervention for human diseases, we made the following discoveries that establish that recombinant MG53 can be used as a therapeutic agent to treat I/R injury in the heart:

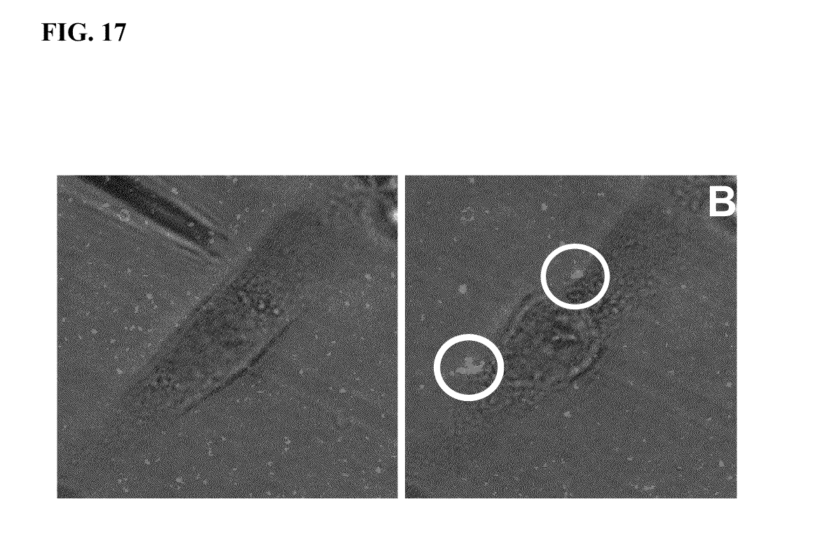

First, we discovered that acute injury of the cell membrane of muscle cells leads to exposure of a signal to the extracellular space that can be detected by MG53, allowing recombinant MG53 to repair membrane damage when provided in the extracellular space (FIG. 17).

Second, we have extensive studies to show that recombinant MG53 purified from E. coli retains efficient membrane repair function, supporting the therapeutic value of targeting MG53 in heart injury and other human diseases.

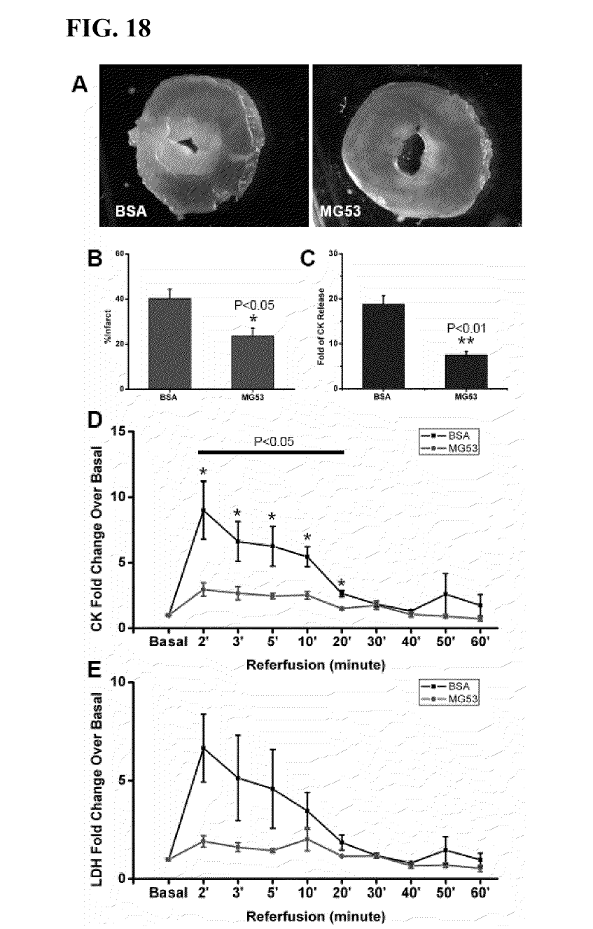

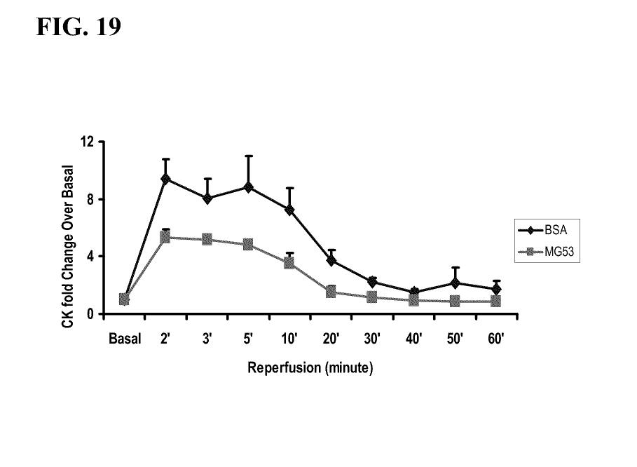

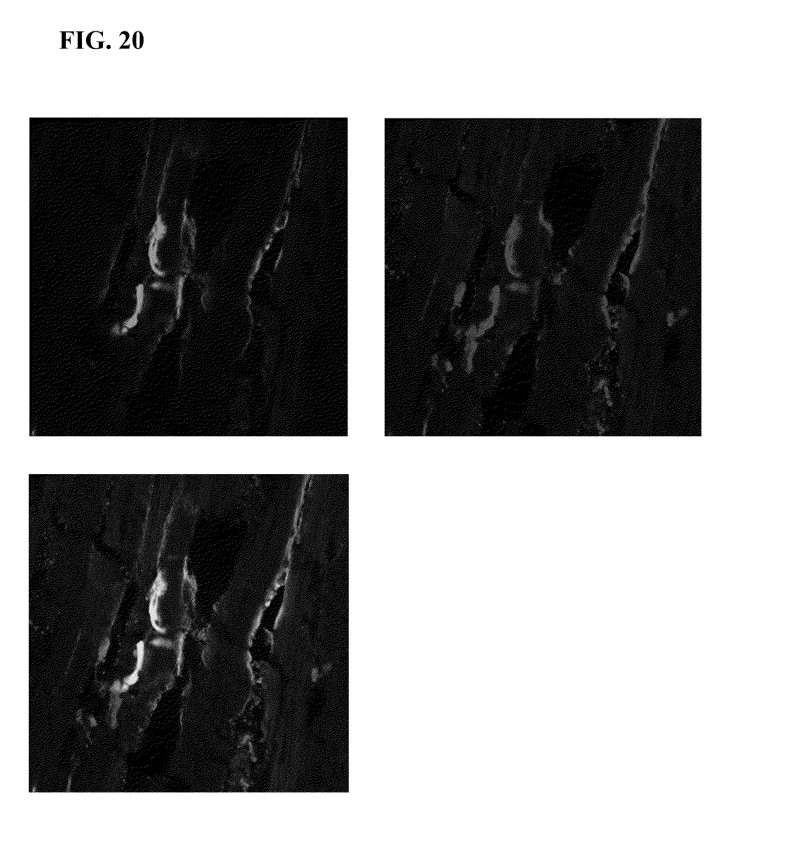

Third, we have data that indicates that intravenous delivery of recombinant MG53 can protect the heart from IR injury when applied wither before (FIG. 18) or after (FIG. 19, 20) reperfusion injury is initiated.

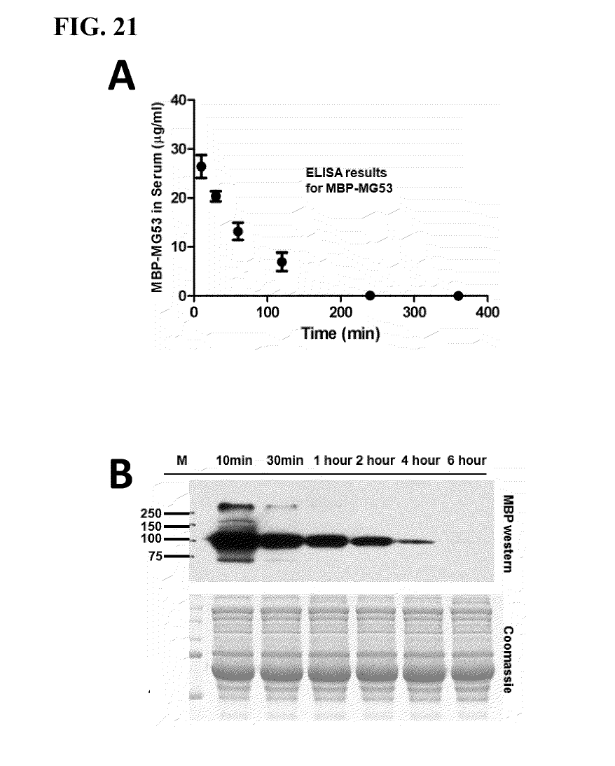

Fourth, we have now determined the pharmacokinetic (PK) properties of recombinant MG53 in the mouse serum. We found that MG53 remains in the serum for ˜4 hrs following tail-vein injection (FIG. 21). These results will allow the use of recombinant MG53 as a therapy for I/R injury in the heart as a short window of treatment is necessary in this application.

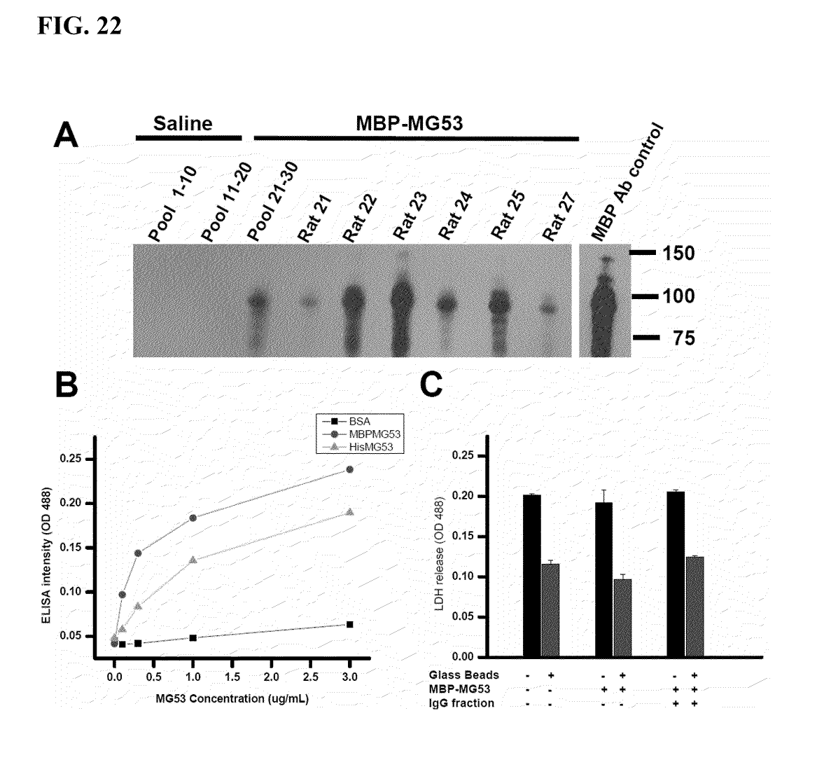

Fifth, as most protein therapeutics result in some immunogenic response in patients treated with the protein. Therefore, we tested if application of recombinant MG53 resulted in the generation of antibodies in rats and if any antibodies generated could block the function of recombinant MG53 (FIG. 22). Intertracheal application of MG53 does produce antibodies in the treated rats, however purified fractions of these antibodies do not block the membrane repair capacity of MG53.

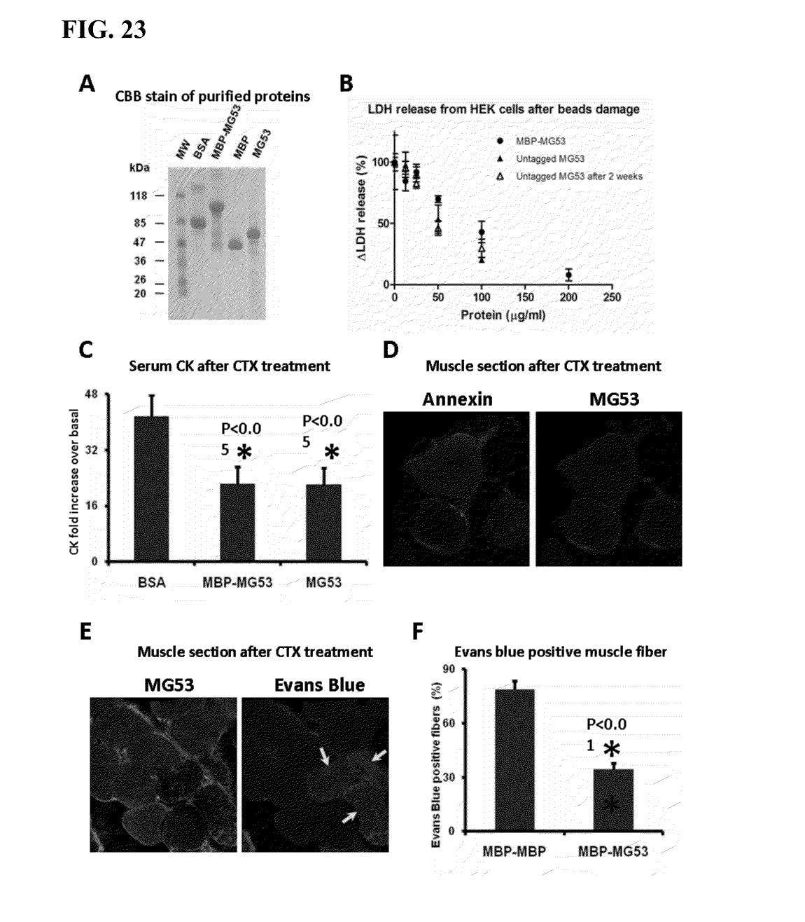

Sixth, to effectively use recombinant MG53 as a therapy for cardiac I/R injury the immunoaffinity tag used for protein isolation must be removed and the protein must remain active. Untagged recombinant MG53 protein is stable and effective at prevention of muscle cell membrane damage (FIG. 23). A part of our continuing developmental effort towards the use of recombinant MG53 protein in treatment of damage to striated muscle tissues we have produced large quantities of untagged MG53 (MG53) by removing the maltose binding protein (MBP) immunoaffinity tag from the protein expressed in E. coli (MBP-MG53).

The data presented here shows direct proof-of-principal data to support the invention that recombinant human MG53 protein can be used to protect against ischemia-reperfusion injury to the heart.

Exemplary Methods

As would be understood by those of skill in the art, certain quantities, amounts, and measurements are subject to theoretical and/or practical limitations in precision, which are inherent to some of the instruments and/or methods. Therefore, unless otherwise indicated, it is contemplated that claimed amounts encompass a reasonable amount of variation.

Identification and cloning of MG53—The preparation and screening of a mAb library for microsomal proteins of rabbit skeletal muscle were described previously. The preparation of mAb5259 (IgG1 subclass) and immunoaffinity purification was carried out as described previously (21). Purified MG53 was subjected to amino acid sequence analysis and all sequences determined were encoded in the rabbit MG53 cDNA (data not shown). Homology searches in the databases found mouse and human MG53 using the rabbit partial amino acid sequences. An exon region of the mouse MG53 gene was amplified from mouse genomic DNA, and rabbit and mouse skeletal muscle libraries were screened using the ³²P-labeled exon fragment to yield full-length cDNAs.

Immunohistochemical and Immunostaining analysis—Immunochemical analyses using mAb5259 were carried out as described previously Immunoelectron-microscopy using secondary antibody conjugated with 15 nm gold particles was conduced as described previously.

Cell culture—The C2C12 murine myoblast cell line used for all studies was purchased from the American Type Culture Collection (Manassas, Va.). Cells were grown in a humidified environment at 37° C. and 5% CO₂ in DMEM medium for C2C12 or Ham's F12 medium for CHO cells supplemented with 10% fetal bovine serum, 100 units/ml penicillin and 100 ug/ml streptomycin. In order to induce myotube differentiation, C2C12 myoblasts were grown to confluence and the medium was switched to DMEM containing 2% horse serum, penicillin (100 U/ml), streptomycin (100 μg/ml). For transient transfections, C2C12 myoblasts or CHO cells were plated at 70% confluence in glass-bottom dishes. After 24 hours, cells were transfected with plasmids described above using GeneJammer reagent (Stratagene). Cells were visualized by live cell confocal imaging at 24-48 hours after transfection or at times indicated for individual experiments. In some experiments, C2C12 myoblasts were allowed to differentiate into myotubes for the indicated time before observation.

Plasmids construction—The full-length mouse MG53 cDNA and associated truncation mutants were generated by PCR using the primers described in supplemental table 1. For construction of pCMS-MG53, after digestion by the appropriate restriction enzymes, the PCR-amplified cDNA was inserted into pCMS-EGFP vector (Invitrogen) at Nhe I/Xba I sites. For construct the GFP-MG53, GFP-TRIM, GFP-SPRY, MG53-GFP, TRIM-GFP and SPRY-GFP, PCR products were inserted into pEGFP-C1 at the XhoI/XbaI sites, or pEGFP-N1 at the XhoI/KpnI sites.

Live cell imaging—To monitor intracellular trafficking of GFP-MG53 either CHO or C2C12 cells were cultured in glass-bottom dishes (Bioptechs Inc.) and transfected with the plasmids described above. Fluorescence images (512×512) were captured at 3.18 s/frame using a BioRad 2100 Radiance laser scanning confocal microscope with a 63×1.3NA oil immersion objective.

RNAi assay—The target sequence for shRNA knockdown of MG53 is at position 622-642 (GAG CTG TCA AGC CTG AAC TCT) in the mouse MG53 cDNA. For caveolin-3, the target sequence is at position 363-380 (GAC ATT CAC TGC AAG GAG ATA). Complementary sense and antisense oligonucleotides were synthesized. To construct the MG53 shRNA and control plasmids, annealed oligonucleotides were inserted into psiRNA-hH1GFPzeo G2 (InvivoGene) at the Acc 65I/Hind III restriction enzyme sites. For caveolin-3 shRNA and control plasmids, annealed oligonucleotides were inserted into pRNAiDsRed vector (BD Biosciences) at the EcoR I/BamH I restriction enzyme sites. Each vector has as independent fluorescent protein expression cassette (green or red) to act as markers of cell transfection. All plasmids were confirmed by direct sequencing with flanking primers and the down-regulation of MG53 and caveolin-3 protein expression was examined by Western blot analysis.

Western blot and Co-immunoprecipitation—Immunoblots were using standard techniques. Briefly, C2C12 or CHO cells were harvested and lysed with ice-cold modified RIPA buffer (150 mM NaCl, 5 mM EDTA, 1% NP40, 20 mM Tris-HCl, pH 7.5) in the presence of a cocktail of protease inhibitors (Sigma). 20 μg of total protein were separated on a 4-12% SDS-polyacrylamide gel. A standard protocol was used for co-immunoprecipitation studies of MG53 and interacting proteins, e.g., Caveolin-3. In brief, skeletal muscle tissue or C2C12 myotubes were lysed in 0.5 ml modified RIPA buffer. The whole cell lysate (500 μg) was incubated overnight with 5 μg polyclonal anti-MG53 (polyclonal antibody), or anti-caveolin-3 antibody (mAb). As a negative control, 500 μg whole cell lysate was incubated with 5 μg normal rabbit and mouse IgG and processed as described above. The immune complexes were collected on protein G-Sepharose beads by incubating for 2 hours and washed four times with RIPA buffer.

Animals Adult male Sprague-Dawley rats, MG53-deficient mice and wild type control mice were maintained and housed in the Center for Experimental Animals (an AAALAC accredited experimental animal facility) at Robert Wood Johnson Medical School, Piscataway, N.J. USA or Peking University, Beijing, China. All procedures involving experimental animals were performed in accordance with protocols approved by the Committee for Animal Research of Peking University, China, and from the animal facility at National Institute on Aging of the NIH, USA, and conformed to the Guide for the Care and Use of Laboratory Animals (NIH publication No. 86-23, revised 1985).

Isolated mouse heart preparation Adult MG53-knockout⁷ and wild type littermate control mice (20 to 30 g) were anesthetized by intraperitoneal (i.p.) injection of pentobarbital (70 mg/kg). After the chest was opened, the heart was excised rapidly and placed in ice-cold Krebs-Henseleit (K-H) perfusion buffer before being mounted on a Langendorff apparatus for perfusion at 37° C. with K-H buffer at a constant pressure (100 cm H₂O) and equilibrated with 95% O₂/5% CO₂. Global ischemia was induced by cessation of perfusion for 30 min, followed by reperfusion for 120 min. IPC was achieved by two cycles of 5 min of ischemia followed by 5 min of reperfusion prior to the more sustained ischemia/reperfusion that caused myocardial infarction.

Rat in vivo myocardial ischemia/reperfusion Male Sprague-Dawley rats weighing 200-250 g were anesthetized with pentobarbital (40 mg/kg, i.p.) and ventilated via a tracheostomy on a Harvard rodent respirator. A midline sternotomy was performed, and a reversible coronary artery snare occluder was placed around the left anterior descending coronary artery. Myocardial IR was performed by tightening the snare for 45 min and then loosening it for 12 h (for RNA extraction) or 24 h (for protein extraction and infarct size measurement). IPC was induced by 4 episodes of 10 min ischemia followed by 5 min reperfusion before the 45-min ischemia. Blood samples for LDH measurement were collected 4 h after the reperfusion.

Measurement of myocardial infarct size To measure the infarct size, isolated perfused hearts were frozen at −80° C. for 10 min and cut into slices, which were then incubated in a sodium phosphate buffer containing 1% 2,3,5-triphenyl-tetrazolium chloride for 15 min to visualize the unstained infarcted region. Infarct and LV areas were determined by planimetry with Image/J software from NIH. The infarct size was calculated as infarct area divided by LV area. To measure the infarct size of rat hearts in vivo, at the end-point, the animals were anesthetized with sodium pentobarbital (50 to 100 mg/kg i.p. to effect) and heparinized (400 USP U/kg, i.p.). The heart was excised and the ascending aorta was cannulated (distal to the sinus of Valsalva), then perfused retrogradely with Alcian blue dye (0.05% solution) to visualize the area at risk (AAR). The coronary artery was re-occluded at the site of occlusion before perfusion with Alcian blue solution. The subsequent procedures were the same as those for ex vivo hearts. The infarct size was calculated as infarct area divided by AAR.

Histology of heart Hearts were fixed in 4% parafomaldehyde (pH 7.4) overnight, embedded in paraffin, and serially sectioned into 5-μm slices. Standard hematoxylin and eosin staining or immunofluorescence was performed with these slices.

Determination of myocardial injury by LDH release The effluent from the isolated perfused heart was accumulated for every 5 min of reperfusion. Blood samples were collected 4 h after reperfusion from rats subjected to IR, and centrifuged for 10 min at 3000 rpm for serum. LDH was spectrophotometrically assayed using a kit from Sigma Chemical Co. LDH activity was expressed as units per liter.

Apoptosis and cell viability assays Cardiomyocyte apoptosis was detected by DNA laddering assay as previously described²⁰. In addition, an ATP assay was used as an independent measure of cell viability, as previously described²¹. Using a luminescence-based commercial kits (Promega) and a luminescence plate reader, we analyzed cellular ATP levels according to the instructions in the operation manual.

TUNEL staining of myocardial sections A CardioTACS™ in situ apoptosis detection kit (#TA5353; R&D Systems Inc, Minneapolis, Minn.) was used to assess DNA fragmentation in myocardial sections. For each sample, two slides were stained. From each slide, 16 10× fields of view were digitized for analysis. TUNEL-positive nuclei and total nuclei were then counted for each image, tallied for each slide, and averaged for each sample. Investigators were blinded to the treatment of the animals in all measurements.

Isolation, culture and adenoviral infection of neonatal rat ventricular myocytes Ventricular myocytes were isolated from 1-day-old Sprague-Dawley rats by methods described previously²⁰. Adenovirus-mediated gene transfer was implemented after 24 h quiescence in serum-free DMEM following 48 h culture in DMEM containing 10% FBS. Infection of cells with an adenoviral vector expressing either GFP-MG53 or GFP was described previously⁷.

For hypoxia, cardiomyocytes were cultured in RPMI 1640/5% FBS for 48 h after infection with adenovirus for 24 h. Then, the medium was changed to serum-free RPMI 1640 saturated with 95% N₂/5% CO₂, and cells were placed in a 37° C. airtight box saturated with 95% N₂/5% CO₂ for 3-24 h. O₂ concentrations were <0.1% (Ohmeda oxygen monitor, type 5120). For normoxic controls, culture medium was changed to RPMI1640/5% FCS/10% HS, and cells were placed in a 37° C./5% CO₂ incubator for 3-24 h before analysis.

Real-time PCR—Quantitative real-time PCR was performed on a Bio-Rad iQ5 multicolor real-time PCR detection system in combination with SYBR Green (Roche Applied Science, Mannheim, Germany). The following primer pairs were used for quantitative real-time PCR: 18S RNA, 5′-GGA AGG GCA CCA CCA GGA GT-3′ (forward) and 5′-TGC AGC CCC GGA CAT CTA AG-3′ (reverse). The primers for MG53 were 5′-CGAGCAGGACCGCACACTT-3′ (forward) and 5′-CCAGGAACATCCGCATCTT-3′ (reverse). Amplification was performed as follows: 94° C. for 30 s and 30 cycles at 94° C. for 30 s, 55° C. for 30 s, and 72° C. for 30 s. The cycle number at which the emission intensity of the sample rose above baseline was referred to as Ct (threshold cycle) and was proportional to target concentration. Data presented are the average of at least 4 independent experiments.

Gene silencing through RNA interference—For gene silencing assay, shRNAs comprising a 19 bp stem and 4 bp loop structure were designed against a unique region of mouse MG53 or rat CaV3 and subcloned into the pAd/BLOCK-iTTMDEST vector (Invitrogen). The sequence of MG53-shRNA was GAGCTGTCAAGCCTGAACTCT, while the sequences of CaV3 shRNA was GACATTCACTGCAAGGAGATA. The sequence of the scramble-shRNA was GCCTGCCGTCCAAAGTTGTAA. Adenovirus expressing GFP-MG53 fusion protein was packaged using the Stratagene Adeasy system. Adenoviruses expressing CaV3-shRNA, or the scramble-shRNA were generated in HEK293 cells using the pAd/BLOCK-iTTMDEST vector adenoviral RNAi expression system, according to the manufacturer's protocol (Invitrogen). The efficiency of gene knockdown was assessed by Western blotting and functional studies at 72 h after adenoviral shRNA transfection.

Materials—Antibodies reacting with phosphorylated Akt, total Akt, phosphorylated GSK3β, total GSK3 and GAPDH were from Cell Signaling Technology. The antibodies reacting with •-actin and •-tubulin were from Santa Cruz. The antibody reacting with CaV3 was from Abcam. The antibody reacting with p85-PI3K was from Upstate. MG53 polyclonal antibody was described previously⁷. Unless indicated otherwise, all chemicals were from Sigma.

Statistical analysis Data are expressed as the mean±s.e.m. Statistical comparisons used one-way analysis of variance, followed by Bonferroni's procedure for multiple-group comparisons. p<0.05 was considered statistically significant.

Echocardiographic evaluation of cardiac morphology and function in MG53-deficient or wt mice—We first trained mice on 2 or 3 separate occasions by picking them up by the nape of the neck and holding them firmly in the palm of one hand in the supine position, with the tail held tightly between the last two fingers. The left hemithorax was shaved, and transthoracic echocardiography was performed using a Doppler echocardiographic system (GE vivid7) equipped with a 13 MHz linear transducer (GE i13L). The heart was first imaged using the two-dimensional mode in the parasternal long-axis and short-axis views. The short-axis views, including papillary muscles, were used to position the M-mode cursor perpendicular to the ventricular septum and LV posterior wall. Images obtained during training sessions were not recorded. Once the mice were trained, images were stored in digital format on a magnetic optical disk for review and analysis. Measurements of the LV end-diastolic diameter (LVEDD) were taken at the time of the apparent maximal LV diastolic dimension, whereas measurements of the LV endsystolic diameter (LVESD) were taken at the time of the most anterior systolic excursion of the posterior wall. LVEF was calculated by the cubic method: LVEF (%)=(LVEDD)³−(LVESD)³/(LVEDD)³, and LVFS was calculated by FS (%)=(LVIDED−LVIDES)/LVIDED×100. The data are averaged from 5 cardiac cycles.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims.

1. A method of treating and/or preventing cardiac injury comprising administering a therapeutic composition comprising an effective amount of an agent that modulates at least one of MG53 activity, MG53 expression, or the MG53 signaling cascade in a cardiac cell, wherein the composition is effective in treating and/or preventing cardiac injury.

2. The method of claim 1, wherein the agent is at least one of an MG53 polypeptide; an MG53 receptor polypeptide; a nucleic acid encoding an MG53 polypeptide; a nucleic acid encoding an MG53 receptor polypeptide; an inhibitory or antisense RNA specific for a nucleic acid encoding MG53, an MG53 receptor, caveolin-3, PI3K, Akt, GSK3•, or ERK 1/2; or an agonist or antagonist of MG53, an MG53 receptor, caveolin-3, PI3K, Akt, GSK3•, ERK 1/2 or MPTP.

3. The method of claim 1, wherein the effective amount is from 0.1 mg/kg and 1000 mg/kg body weight/day.

4. The method of claim 2, wherein the agonist of MG53 activity comprises at least one of phosphotidylserine, zinc or zinc salt, Zn-1-hydroxypyridine-2-thine (Zn-HPT), notoginsing, an oxidizing agent, thimerosal, or combination thereof.

5. The method of claim 1, wherein the agent is a stem cell capable of differentiation into a cardiac myocyte, wherein the stem cell has been modified such that it demonstrates enhanced activity or expression of MG53.

6. The method of claim 1, wherein the composition further comprises a pharmaceutically acceptable carrier or excipient.

7. The method of claim 1, wherein the cardiac injury comprises cardiac cell or myocardial tissue injury due to at least one of cardiovascular disease, cardiac ischemia/reperfusion injury, hypoxic injury, heart failure, or a combination thereof.

8. The method of claim 1, wherein the method further comprises performing an ischemic preconditioning (IPC) step at a time prior to, and/or approximately contemporaneous with, and/or subsequent to the administration of the therapeutic composition.

9. The method of claim 6, wherein the cardiac injury comprises cardiac cell or myocardial tissue injury due to cardiac ischemia/reperfusion injury.

10. The method of claim 2, wherein the polypeptide has the amino acid sequence of at least one of SEQ ID NOs.: 1, 3, 5, 9, 10, 11, 12, 13, 14, 15, or 16 or bioactive portion thereof.

11. Method of claim 1, wherein the composition is administered extracellularly or systemically in combination with a pharmaceutically acceptable excipient, wherein the composition is effective in treating or preventing injury of cardiac tissue.

12. A method of treating cardiac ischemia/reperfusion injury comprising administering and effective amount of a therapeutic composition comprising an MG53 polypeptide or MG53 nucleic acid, wherein the composition further includes a pharmaceutically acceptable excipient, and wherein the composition is effective in treating cardiac ischemia/reperfusion injury.

13. The method of claim 12, wherein the effective amount is from 0.1 mg/kg and 1000 mg/kg body weight/day.

14. The method of claim 12, wherein the therapeutic composition further comprises an agonist of MG53 activity.

15. The method of claim 14, wherein the agonist is at least one of phosphotidylserine, zinc or zinc salt, Zn-1-hydroxypyridine-2-thine (Zn-HPT), notoginsing, an oxidizing agent, thimerosal, or a combination thereof.

16. A method of screening for a compound useful for treating cardiac ischemic/reperfusion injury comprising the steps of: providing a myocardial cell or cardiac cell expressing a nucleic acid encoding a polypeptide having an amino acid sequence with at least 70% homology to an MG53 polypeptide; providing a library of test compounds; treating the cell with the test compound; inducing ischemic/reperfusion damage to the membrane of the cardiac cell; measuring for a change in MG53 expression, activity, or amount and the ability of the cell to repair damage to a membrane; and comparing the treated cell versus an untreated cell, wherein an agent that is capable of modulating MG53 expression, activity or amount is indicative of an agent useful for treating cardiac ischemic/reperfusion injury.


SEQUENCE LISTING (16 sequences)

SEQ ID NO: 1 - protein, 477 aa, Homo sapiens
  misc_feature (1)..(477): Human MG53 Polypeptide
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDCEAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 2 - DNA, 1434 bp, Homo sapiens
  misc_feature (1)..(1434): Human MG53 cDNA
atgtcggctgcgcccggcctcctgcaccaggagctgtcctgcccgctgtgcctgcagctgttcgacgcgcccgtgacagccgagtgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaagacacagctgccacagcagaaactgcagctgcaggaggcatgcatgcgtaaggagaagagtgtggctgtgctggagcatcagctggtggaggtggaggagacagtgcgtcagttccggggggccgtgggggagcagctgggcaagatgcgggtgttcctggctgcactggagggctccttggactgcgaggcagagcgtgtacggggtgaggcaggggtcgccttgcgccgggagctggggagcctgaactcttacctggagcagctgcggcagatggagaaggtcctggaggaggtggcggacaagccgcagactgagttcctcatgaaatactgcctggtgaccagcaggctgcagaagatcctggcagagtctcccccacccgcccgtctggacatccagctgccaattatctcagatgacttcaaattccaggtgtggaggaagatgttccgggctctgatgccagcgctggaggagctgacctttgacccgagctctgcgcacccgagcctggtggtgtcttcctctggccgccgcgtggagtgctcggagcagaaggcgccgccggccggggaggacccgcgccagttcgacaaggcggtggcggtggtggcgcaccagcagctctccgagggcgagcactactgggaggtggatgttggcgacaagccgcgctgggcgctgggcgtgatcgcggccgaggccccccgccgcgggcgcctgcacgcggtgccctcgcagggcctgtggctgctggggctgcgcgagggcaagatcctggaggcacacgtggaggccaaggagccgcgcgctctgcgcagccccgagaggcggcccacgcgcattggcctttacctgagcttcggcgacggcgtcctctccttctacgatgccagcgacgccgacgcgctcgtgccgctttttgccttccacgagcgcctgcccaggcccgtgtaccccttcttcgacgtgtgctggcacgacaagggcaagaatgcccagccgctgctgctcgtgggtcccgaaggcgccgaggcctga

SEQ ID NO: 3 - protein, 477 aa, Mus musculus
  misc_feature (1)..(477): Mouse MG53
MSAAPGLLRQELSCPLCLQLFDAPVTAECGHSFCRACLIRVAGEPAADGTVACPCCQAPTRPQALSTNLQLSRLVEGLAQVPQGHCEEHLDPLSIYCEQDRTLVCGVCASLGSHRGHRLLPAAEAQARLKTQLPQQKMQLQEACMRKEKTVAVLEHQLVEVEETVRQFRGAVGEQLGKMRMFLAALESSLDREAERVRGDAGVALRRELSSLNSYLEQLRQMEKVLEEVADKPQTEFLMKFCLVTSRLQKILSESPPPARLDIQLPVISDDFKFQVWKKMFRALMPALEELTFDPSSAHPSLVVSSSGRRVECSDQKAPPAGEDTRQFDKAVAVVAQQLLSQGEHYWEVEVGDKPRWALGVMAADASRRGRLHAVPSQGLWLLGLRDGKILEAHVEAKEPRALRTPERPPARIGLYLSFADGVLAFYDASNPDVLTPIFSFHERLPGPVYPIFDVCWHDKGKNAQPLLLVGPEQEQA

SEQ ID NO: 4 - DNA, 1434 bp, Mus musculus
  misc_feature (1)..(1434): Mouse MG53 cDNA
atgtcggctgcacccggccttctgcgtcaggaactgtcctgcccactgtgcttgcagctgttcgatgcgccagtgacggctgagtgtggccacagtttctgccgtgcctgcctgatccgggtggcaggggagcctgctgcggacggcacagttgcctgtccctgttgtcaggcacctacacggccgcaggctctaagcactaacctccagttgtcacgccttgtggagggtttggcgcaagtgccccaaggccactgcgaggaacacctggatccactgagcatctactgcgagcaggaccgcacacttgtgtgtggtgtgtgtgcctcgctcggttctcaccgtggtcatcgtctcctgcctgccgctgaagcccaagcacgcctcaagacacagcttccacagcagaagatgcagctgcaggaggcatgcatgcgcaaggagaagactgtagcggtgctggagcatcagctggtggaggtggaggagacagtgcgccagttccggggagctgtcggggagcagctggggaagatgcggatgttcctggctgccctagaaagttctctggaccgtgaagcagaaagggttcggggtgatgctggggttgccttgcgtcgggagctgtcaagcctgaactcttacctagagcaactgaggcagatggagaaggtgctggaggaggtggctgacaagccacagacagaattcctcatgaaattctgcctggtaaccagcaggctgcagaagatcctgtcagagtcaccaccaccggcaaggctagatatccagctgcctgtcatctcagatgacttcaaattccaggtgtggaagaagatgttccgggctctgatgccagcgctggaggaactgacttttgaccccagctctgcgcacccgagcctggtggtgtcctcctctggtcgccgagtggagtgctcagaccagaaggcgccgccagcgggagaagacacgcgtcagttcgacaaggcagtagcggtggtggcgcagcagctgctgtcacagggcgagcactattgggaggtggaggtgggcgacaaaccacgctgggccctgggagtgatggcggctgacgcttcccgccgtggccggctgcacgcggtgccctcacaggggctgtggctgctgggtctgcgcgatggcaagatcctggaggcgcacgtggaggccaaggagccgcgggcactgcgcaccccagagaggcctccggcgcgcattggcctctacctaagcttcgcagatggcgtcctggctttctatgatgcgagcaaccccgacgtacttacgccaatcttttctttccacgagcgtctgcccgggccggtgtaccccatctttgacgtgtgctggcacgacaagggcaagaatgcccagcccctgctgcttgtggggccggagcaggaacaggcctga

SEQ ID NO: 5 - protein, 477 aa, Oryctolagus cuniculus
  misc_feature (1)..(477): Rabbit MG53
MSAAPGLLHQELSCPLCLQLFDAPVTAECGHSFCRACLSRVAGEPAADGTVNCPCCQAPTRPQALSTNLQLARLVEGLAQVPQGHCEEHLDPLSIYCEQDRVLVCGVCASLGSHRGHRLLPAAEAHSRLKTQLPQQKLQLQEASMRKEKSVAVLEHQLTEVEETVRQFRGAVGEQLGKMRVFLAALEGSLDREAERVRSEAGVALRRELGGLHSYLEQLRQMEKVLEEVADKPQTEFLMKYCLVTSRLQKILAESPPPARLDIQLPIISDDFKFQVWRKMFRALMPALEELTFDPSSAHPSLVVSPTGRRVECSEQKAPPAGDDARQFDKAVAVVAQQLLSDGEHYWEVEVGDKPRWALGVMASEASRRGRLHAVPSQGLWLLGLRDGKTLEAHVEAKEPRALRTPERRPTRLGLYLSFGDGVLAFYDASDADALELLFAFRERLPGPVYPFFDVCWHDKGKNAQPLLLVGPDGQEA

SEQ ID NO: 6 - DNA, 1434 bp, Oryctolagus cuniculus
  misc_feature (1)..(1434): Rabbit MG53 cDNA
atgtcggccgcgcccggcctcctgcaccaggagctgtcttgcccgctgtgcctgcagctgttcgacgcgcccgtgacagccgagtgcggccacagtttctgccgcgcctgcctgagccgcgtggcgggggagccggcggccgatggcaccgtgaactgcccgtgctgccaggcgcccacgcggccgcaggcgctcagcaccaacctgcagctggcgcgcctggtggaggggctggcgcaggtgccgcagggccactgcgaggagcacctggacccgctgagcatctactgcgagcaggaccgcgttctcgtgtgcggcgtgtgcgcctcgctcggctcgcaccgcggccaccgcctgctgcccgccgccgaggcccactcgcgtctcaagacgcagctgccccagcagaagctgcagctgcaggaggcgagcatgcgcaaggagaagagcgtggccgtgctggagcaccagctcacggaggtggaggagacagtgcgtcagttccggggggcagtgggggagcagctgggcaagatgcgggtgttcctggccgccctggagggctccctggaccgcgaggcagaacgtgtgcggagcgaggcgggggtggccttgcggcgggagctggggggcctccactcgtacctggagcagctgcggcagatggagaaggtgttggaggaggtggctgacaagccacagaccgagttccttatgaaatattgcctggtgaccagcaggctgcagaagatcctggcggagtcgccaccacctgctcgtctggacatccagctgcccatcatttcagatgacttcaaattccaggtgtggaggaagatgttccgggctctgatgccagcgctggaggagctgacctttgacccgagctccgcgcacccgagcctcgtggtgtcacccacgggccgccgagtggagtgctcggagcagaaggcgccgcccgccggggacgacgcgcgccagttcgacaaggctgtggccgtggtggcgcagcagctgctgtccgacggcgagcactactgggaggtggaggtgggcgacaagccgcgctgggcgctgggcgtgatggcctccgaggcgagccgccgtggccggctgcacgccgtgccctcacagggtttgtggctgctggggctgcgcgacggcaagaccctggaggcgcacgtggaggccaaggagccgcgcgcgctgcgcaccccggagcggcggcccacgcgcctcggcctctacctcagcttcggcgatggcgtgctcgccttctacgacgccagcgacgccgacgcgctcgagctgctgtttgctttccgcgagcgcctgcccgggcccgtgtaccccttcttcgacgtgtgctggcatgacaagggcaagaatgcgcagccgctgctgctcgtggggccggatggccaggaggcctga

SEQ ID NO: 7 - protein, 477 aa, Homo sapiens
  MUTAGEN (29)..(29): C29L/C242A
MSAAPGLLHQELSCPLCLQLFDAPVTAELGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDCEAERVRGEAGVALRRELGSLNSYLEQLRQMEKVLEEVADKPQTEFLMKYALVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 8 - protein, 477 aa, Didelphis sp.
  PEPTIDE (1)..(477): Opossum MG53
MSGAPALMQGMYQDLSCPLCLKLFDAPITAECGHSFCRNCLLRLAPDPQAGTVLCPSCQAPTKPDGLNTNQQLARLVESLAQVPQGHCEEHLDPLSVYCEQDRALICGVCASLGKHRGHSVVTAAEAHQRMKKQLPQQRLQLQEACMRKEKTVALLDRQLAEVEETVRQFQRAVGEQLGVMRAFLAALESSLGKEAERVTGEAGTALKAERRIVTSYLDQLQQMEKVLDEVTDQPQTEFLRKYCLVISRLQKILAESPPAARLDIQLPIISDDFKFQVWRKMFRALMPGMEVLTFDPASAHPSLLVSPSGRRVECVEQKAPPAGDDPQQFDKAVALVAKQQLSEGEHYWEVEVGDKPRWGLGLISADVSRRGKLHPTPSQGFWMLGLREGKVYEAHVESKEPKVLKVDGRPSRIGLYLSFRDGVLSFYDASDLDNLLPLYAFHERLPGPVYPFFDVCWHDKGKNAQPLLLLGPDGEQ

SEQ ID NO: 9 - protein, 477 aa, Canis sp.
  PEPTIDE (1)..(477): Dog MG53
MSAAPGLLHQELSCPLCLQLFDAPVTAECGHSFCRACLSRVAGEPAADGTVPCPCCQALTRPQALSTNQQLARLVEGLAQVPQGHCEEHLDPLSIYCEQDRALVCGVCASLGSHRGHRLLPAAEAHARLKTQLPQQKLQLQEACMRKEKSVALLEHQLMEVEEMVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPVISDDFKFQVWRKMFRALMPVTKELTFDPSSAHPSLVLSPSGRRVECSDQKAPPAGEDPCQFDKAVAVVAQQVLSDGEHYWEVQVGEKPRWALGVIAAQASRRGRLHAVPSQGLWLLGLRDGKILEAHVEAKEPRALRTPERRPTRIGIYLSFGDGVLSFYDASDPDALELLFAFHERLPGPVYPFFDVCWHDKGKNAQPLLLVGPDGEEA

SEQ ID NO: 10 - protein, 477 aa, Pan troglodytes
  PEPTIDE (1)..(477): Chimpanzee MG53
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 11 - protein, 477 aa, Macaca mulatta
  PEPTIDE (1)..(477): Rhesus Monkey MG53
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVEVGDKPRWALGVIAAEGPRRGRLHAVPSQGLWLLGLREGKILEAHVEAKEPRALRSPERRPTRIGLYLSFGDGVLSFYDASDADALVPLFAFHERLPGPVYPFFDVCWHDKGKNSQPLLLVGSEGAEA

SEQ ID NO: 12 - protein, 482 aa, Bos sp.
  PEPTIDE (1)..(482): Bovine MG53
MSAAPGLLHQELSCPLCLQLFDAPVTAECGHSFCRACLSRVAGEPAADGTVLCPSCQAPTRPQALSTNLQLARLVEGLAQVPQGHCEEHLDPLSIYCEQDRALVCGVCASLGSHRGHRLLPAAEAHARLKTQLPQQKMQLQEACMRKEKSVALLEHQLLEVEETVRQFRGAVGEQLGKMRLFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPARQELTFDPSTAHPSLVLSNSGRCVECSEQKAPPAGEDPRQFDKAVAVVTHQLLSEGEHYWEVEVGDKPRWALGVIGAQAGRRGRLHAVPSQGLWLLGLRDGKILEAHVEAKEPRALRTPERRPTRIGIYLSFGDGVLSFYDASDPDALELLFAFHERLPGPVYPFFDVCWHDKGKNAQPLLLVGPEVSGGSGSEA

SEQ ID NO: 13 - protein, 477 aa, Rattus sp.
  PEPTIDE (1)..(477): Rat MG53
MSTAPGLLRQELSCPLCLQLFDAPVTAECGHSFCRACLIRVAGEPADDGTVACPCCQASTRPQALSTNLQLARLVEGLAQVPQGHCEEHLDPLSIYCEQDRTLVCGVCASLGSHRGHRLLPAAEAHARLKTQLPQQKAQLQEACMRKEKSVAVLEHQLVEVEETVRQFRGAVGEQLGKMRMFLAALESSLDREAERVRGEAGVALRRELSSLNSYLEQLRQMEKVLEEVADKPQTEFLMKFCLVTSRLQKILSESPPPARLDIQLPVISDDFKFQVWKKMFRALMPELEELTFDPSSAHPSLVVSASGRRVECSEQKAPPAGEDTCQFDKTVAVVAKQLLSQGEHYWEVEVGDKPRWALGVMAADASRRGRLHAVPSQGLWLLGLRDGKILEAHVEAKEPRALRTPERPPARIGLYLSFADGVLTFYDASNTDALTPLFSFHERLPGPVYPMFDVCWHDKGKNSQPLLLVGPDSEQA

SEQ ID NO: 14 - protein, 477 aa, Xenopus laevis
  PEPTIDE (1)..(477): Xenopus laevis
MSTPQLMQGMQKDLTCQLCLELFRAPVTPECGHTFCQGCLTGVPKNQDQNGSTPCPTCQSPSRPETLQINRQLEHLVQSFKQVPQGHCLEHMDPLSVYCEQDKELICGVCASLGKHKGHNIITASEAFAKLKRQLPQQQVILQEARLKKEKTVAVLDRQVAEVQDTVSRFKGNVKHQLNAMRSYLNIMEASLGKEADKAESAATEALLVERKTMGHYLDQLRQMEGVLKDVEGQEQTEFLRKYCVVAARLNKILSESPPPGRLDIQLPIISDEFKFQVWRKMFRALMPALENMTFDPDTAQQYLVVSSEGKSVECADQKQSVSDEPNRFDKSNCLVSKQSFTEGEHYWEVIVEDKPRWALGIISETANRKGKLHATPSNGFWIIGCKEGKVYEAHTEQKEPRVLRVEGRPEKIGVYLSFSDGVVSFFDSSDEDNLKLLYTFNERFSGRLHPFFDVCWHDKGKNSQPLKIFYPPAEQL

SEQ ID NO: 15 - protein, 477 aa, Xenopus sp.
  PEPTIDE (1)..(477): Xenopus tropicalis MG53
MSTPQLMQGMQKDLTCPLCLELFRAPVTPECGHTFCQGCLTGAPKNQDQNGSTPCPTCQTPSRPETLQINRQLEHLVQSFKQVPKGHCLEHLDPLSVYCEQDKELICGVCASLGKHKGHNIITAAEAYAKLKRQLPQQQVILQEARLKKEKTVAVLDRQVAEVQDTVSRFKGNVKHQLNAMRSYLSIMEASLSKEADNAEHTATEALLVERKTMGHYLDQLRQMDGVLKDVESQEQTEFLRKYCVVAARLNKILAESPPPGRLDIQLPIISDEFKFQVWRKMFRALMPALENLTFDPDTAQQNLVVFSDGKSVECSEQKQSVSDEPNRFDKSNCLVSKESFTEGEHYWEVLVEDKPRWALGVISETANRKGKLHASPSNGFWLIGCKEGKVYEAHTEQKEPRVLRVEGRPEKIGIYLSFSDGVVSFFDSSDEDNIKLLYTFNERFSGRLHPFFDVCWHDKGKNAQPLKIFYPPAEQL

SEQ ID NO: 16 - protein, 101 aa, Human immunodeficiency virus type 1
  PEPTIDE (1)..(101): HIV-1 TAT protein
MEPVDPNLEPWKHPGSQPPTACSKCYCKKCCWHCQLCFLKKGLGISYGRKKRKHRRGTPQSSKDHQNPIPEQPLPIIRGNQTGPKEQKKTVASKAERDLCA